(19) United States
(12) Patent Application Publication
(10) Pub. No.: US 2015/0024452 A1
(43) Pub. Date: Jan. 22, 2015

(54) EXHAUST SCRUBBER

(71) Applicant: Biogas & Electric, LLC, San Diego, CA (US)

(72) Inventors: Michael C. MATELICH, San Diego, CA (US); Seth S. BURNS, La Mesa, CA (US); Ken SCHIFFTNER, Mahwah, NJ (US)

(21) Appl. No.: 14/333,429

(22) Filed: Jul. 16, 2014

Related U.S. Application Data
(60) Provisional application No. 61/847,039, filed on Jul. 16, 2013.
(60) Provisional application No. 61/847,040, filed on Jul. 16, 2013.

(51) Int. Cl. B01D 53/86; C12M 1/107; C12M 1/00; B01D 53/14; C12P 3/00

(52) U.S. Cl. CPC B01D 53/8637; B01D 53/1406; B01D 53/1456; B01D 53/1481; C12P 3/00; C12M 43/04; C12M 21/04
USPC 435/168; 422/169

(57) ABSTRACT

Provided herein are methods, devices and systems for decreasing emissions in exhaust comprising contacting the exhaust with a liquid waste stream from a biogas production unit, the liquid waste stream being contacted with the exhaust, optionally, in a plurality absorbers operatively connected in-line and/or in parallel.

### CROSS REFERENCE

The present application is a non-provisional of, and claims the benefit of U.S. Provisional Application Nos. 61/847,039 and 61/847,040, each of which were filed Jul. 16, 2013 and the entire contents of each which are hereby incorporated by reference.

### BACKGROUND OF THE INVENTION

Ground-level ozone is created through photochemical reaction between oxides of nitrogen (“NOx”), volatile organic compounds (“VOCs”) and sunlight. NOx is emitted by internal combustion engines, and VOCs are generated from gasoline vapors and chemical solvents. NOx and VOCs combine in the atmosphere to create damaging ground level ozone that is a major health and economic concern.

Wet-scrubbing technology has been used in the air pollution control industry to decrease NOx emissions. Traditionally, a caustic liquid with a certain high pH would be injected into the top of such systems and would flow counter-current to the exhaust stream thereby sinking the targeted pollutants into the liquid phase. Conventionally synthetic chemicals are continually injected into the wet scrubbing system to maintain the pH of the caustic liquid. Typically, the liquid used to scrub the exhaust stream creates yet another waste stream that naturally requires management.

### SUMMARY OF THE INVENTION

Described herein is a wet scrubbing technology that reduces NOx and SOx emissions from the exhaust stream of an engine, including as one example a biogas fired internal combustion engine. According to the American Biogas Council, there are an estimated 12,000 sites in the United States available for biogas project development. There are 8,200 farms with a potential for 1,700 MW and 3,899 wastewater treatment plants with a potential for 750 MW. Additionally, there are over 300 biogas projects currently being developed in the US. Presently, there are more than 2,000 biogas producing sites that are operational in the United States, 192 digesters on farms and 1,238 anaerobic digesters at wastewater treatment plants. Emissions regulations governing stationary biogas engines in EPA designated ozone non-attainment areas are very stringent, effectively stopping biogas project development.

Engines generally, and biogas engines in particular, pollute the environment by contaminating air with NOx emissions. Therefore, local, state and federal NOx regulations have established maximum levels of NOx production for biogas engines. For example, the California Energy Commission (CEC) has established guidelines for certification of combined heat and power engine systems, including for example, biogas engines, that limit NOx emission levels pursuant to the Waste, Heat and Carbon Emissions Reduction Act of California. Similarly, the local San Joaquin Air Board has established Rule 4702 that govern NOx emissions of biogas engines. These stringent NOx emission standards are a hindrance to the operation and development of biogas engines.

NOx, however, is not the only pollutant of concern related to anaerobic digestion. California, as an example, is home to about 1,750 dairies and 1.8 million dairy cows, producing in aggregate about 35-40 million tons of manure each year. Manure management consists primarily of converting the raw manure to a form that is acceptable for spreading on agricultural fields. Typically, flush water from animal holding areas is transferred to settling tanks for removal of large solid particles, and then stored in holding ponds. During the holding period, anaerobic bacteria convert carbohydrates, fatty acids, and other high energy organic compounds to methane and carbon dioxide that is then typically released into the atmosphere. The liquid suspension remaining after this stabilization process has reduced odor, reduced pathogens, and is less attractive to flies and other nuisance species. As a result of these processes, large quantities of the greenhouse gas methane are released into the atmosphere. Methane is approximately 21 times more potent than carbon dioxide in its global warming potential, and its relatively short atmospheric half-life of 12 years makes it an excellent candidate for reducing global warming in the short term. Biogas produced by anaerobic digestion of dairy manure contains methane, carbon dioxide and a variety of trace gases including hydrogen sulfide. Collecting and combusting the biogas mixture in a reciprocating internal combustion engine has a two-fold favorable effect on greenhouse emissions. The methane is converted to an equal quantity of carbon dioxide, a gas with lower greenhouse warming potential, and the production of an approximately equal quantity of carbon dioxide from the burning of fossil fuels is avoided. The methane produced and released into the atmosphere by California dairy manure management processes totals 450,000 tons per year. There are an estimated 900 dairies in California alone that will benefit from the technology described herein.

NOx emissions from combustion of biogas generated by anaerobic digestion facilities are a serious problem that significantly impedes expansion of biogas utilization for electricity generation. Nitrogen oxides are one of six criteria pollutants for which the Clean Air Act requires the EPA to set National Ambient Air Quality Standards (NAAQS). While exposure to high levels of nitrogen oxides has human health effects, their adverse environmental effects are primarily due to secondary reactions in the atmosphere. For example, the photochemical reaction of NO₂ with volatile organic compounds (VOCs) produces ground level ozone, an important air pollutant and lung irritant. Under the Clean Air Act, each state must develop a plan describing how it will meet the NAAQS. Most of the state of California, including the San Joaquin Valley that serves as home to most of the state's dairy farms, fall within a NAAQS ozone non-attainment zone. Distributed generation projects in non-attainment zones are considered “minor sources” subject to emissions standards set by the states. For all new distributed generation facilities coming on line after Jan. 1, 2007, the California Air Resource Board (CARB) has set NOx emissions limits of 0.07 lb/MW hr. The current high cost of reducing NOx emissions has effectively suspended many California manure based anaerobic digestion projects. The fraction of U.S. counties lying within ozone non-attainment zones is expected to increase over coming years as increasingly strict NAAQS come into effect. In fact, it is believed by many in the industry that without a more effective type of new catalytic converter or other new technology, exhaust from biogas engines contains levels of NOx too high to legally operate.

Internal combustion engines using biogas (or other fuels) produce a mixture of nitrogen oxides including nitric oxide (NO) and nitrogen dioxide (NO₂) and other NOx gases via oxidation of atmospheric nitrogen. Certain engines also combust high nitrogen fuels such as coal where significant NOx is formed through oxidation of the nitrogen contained within the fuel. Conventional reciprocating engines operating without exhaust gas treatment modalities produce approximately 1.4 pounds of NOx per megawatt hour (MW h) which is 20 times the current CARB standard. The benefits to the water and air quality provided by the subject matter described herein are significant.

The inventors of the subject matter described herein have identified factors limiting the application of wet scrubbing techniques, including cost of both the chemical scrubbing agents, the disposal of used liquid chemical waste streams, and other features described herein. A feature of the subject matter described herein is SOx and NOx reduction systems having reduced installation and operational costs that are capable of complying with regulatory standards for such systems, including as one example the California Air Resource Board's (CARB) NOx standard of 0.07 lbs. of NOx per Megawatt Hour (roughly 2-3 ppm of NOx at 15% O₂ levels). The subject matter described herein enables a biogas project to use a low cost and highly efficient internal combustion engine to produce both electrical and thermal energy without the necessity of resorting to inferior and expensive alternatives such as fuel cells, micro-turbines, upgrading biogas for the pipeline, or gasification.

Provided herein are methods, devices, and systems for the attenuation of NOx emissions from biogas combustion utilizing a liquid waste stream and application of principles of extended absorption, wet chemical scrubbing, and/or selective non-catalytic reduction.

In one aspect, described herein are methods, platforms, devices and the systems comprising: (a) an absorber that comprises a microorganism that is capable of reducing NOx, provided that the absorber is operatively connected to a liquid waste source and an engine exhaust source; and (b) an engine, including as one example, a combustion engine that produces energy and NOx-containing engine exhaust. In some embodiments, the system comprises a biogas production unit that converts waste to a biogas stream and a liquid waste stream. In some embodiments of the systems and methods described herein, provided is a combustion engine, such as a biogas engine. In some embodiments of the subject matter described herein, the system is a wet scrubbing system. In some embodiments of the systems described herein, NOx is reduced by bacteria when engine exhaust is contacted with a liquid waste stream in the absorber(s). In some embodiments of the systems described herein, the system further comprises a quencher that is operatively connected to an outlet from the combustion engine. In some embodiments of the systems described herein, the system further comprises a cooler that is operatively connected to the quencher. In further or additional embodiments of the systems described herein, the system further comprises a second, third, fourth, fifth, or further or additional absorber(s). In one embodiment, the biogas production unit is an anaerobic digester. In further or additional embodiments, the microorganisms comprise bacteria and the bacteria comprises nitrogen or nitrate reducing bacteria. In some embodiments of the systems described herein, the nitrogen or nitrate reducing bacteria is Thiobacillus denbrificans. In some embodiments of the systems described herein, the nitrogen or nitrate reducing bacteria is Micrococcus denbrificans. In a particular embodiment of the system described herein, the nitrogen or nitrate reducing bacteria is Paracoccus denbrificans. In some embodiments of the subject matter described herein, the nitrogen or nitrate reducing bacteria is Pseudomonas. In some embodiments of the system described herein, the bacteria comprises sulfate-reducing bacteria.

In a second aspect, described herein are methods, devices and systems comprising: (a) an absorber that is operatively connected to a liquid waste source and an engine exhaust source; (b) an engine that produces energy and engine exhaust containing NOx; and (c) a fan or blower that is displaced or located within the system. For example, in some embodiments, the fan or blower is displaced or located prior to entrance of the engine exhaust into the absorber so as to reduce positive back-pressure on the engine. In some embodiments, the fan is operatively connected to a quencher, a cooler, the absorber, or a combination thereof. In some embodiments of the systems described herein, the fan is operatively connected to a cooler and the absorber(s). In some embodiments, the system further comprises a second absorber. In further or additional embodiments, one or more fans are operatively connected to one or more absorbers.

In a third aspect, described herein are methods, platforms, devices and systems comprising: (a) an absorber that is operatively connected to an engine exhaust source; (b) a combustion engine that produces energy and engine exhaust containing NOx; and (c) an oxidant source. In some embodiments, the oxidant source is ozone or hydrogen peroxide. In some embodiments, the ozone is operatively connected to the engine exhaust. In some embodiments, provided are at least two absorbers and an oxidant source is operatively connected to each absorber. In some embodiments of the systems described herein, the oxidant source provides a single point, continuous infusion of ozone into the engine exhaust. In further or additional embodiments, less than a stoichiometric mole equivalent of ozone is provided compared to NO (mol: mol). In some embodiments of the systems described herein, a mist eliminator is operatively connected to the oxidant source. For example, in some embodiments the mist eliminator is installed or displaced above packing media within a wet scrubber. In further or additional embodiments, the mist eliminator removes water droplets from the exhaust stream prior to injection of an oxidant, such as ozone or hydrogen peroxide. In further or additional embodiments, the mist eliminator is installed upstream prior to the injection point of oxidant.

In further or additional embodiments of the systems described herein, the mist eliminator comprises a polypropylene mesh material. In further embodiments provided is a fan. In some embodiments, the fan is placed before a cooler within the system. In further or additional embodiments, the fan is placed between the cooler and the absorber. In some embodiments, the fan is placed after the absorber. In some embodiments, the oxidant source is ozone and the temperature of the exhaust at a point of ozone infusion is a temperature up to about 100-120° F.

In a fourth aspect provided herein are methods, platforms devices and systems comprising a wet scrubbing system wherein: a) a biogas production unit converts waste to a biogas stream and a liquid waste stream; b) a combustion engine that utilizes the biogas stream to produce energy and an engine exhaust stream containing NOx and comprising a biogas stream inlet and an engine exhaust stream outlet connected to the engine exhaust inlet of an absorber; and c) an oxidant source that is connected to the engine exhaust stream inlet of the absorber. In some embodiments the oxidant source is connected to the exhaust duct, optionally through an oxidant inlet. In some embodiments, the oxidant source is connected to the exhaust duct through the oxidant inlet prior, for example about 15 feet prior, to the exhaust inlet of the absorber. In some embodiments, a gas mixing device, for example a static mixer, is included in the exhaust duct at the oxidant inlet or between the oxidant inlet and the exhaust inlet of the absorber. In some embodiments of the subject matter described herein, provided are multiple absorbers arranged successively in-line/series and/or in parallel. In some embodiments, the biogas production unit for converting waste to a biogas stream is an anaerobic digester. In some embodiments, a single point, continuous infusion of hydrogen peroxide or ozone is provided to the engine exhaust stream.

In a fifth aspect, described herein is a multistage wet scrubbing system comprising a liquid waste stream inlet, an engine exhaust inlet, a quencher, cooler and an absorber, provided that: (a) a biogas production unit converts waste to a biogas stream and a liquid waste stream, and (b) a combustion engine utilizes the biogas stream to produce energy and an engine exhaust stream containing NOx, provided that a fan is incorporated in-line along an engine exhaust duct. In some embodiments, provided are multiple absorbers arranged successively, for example in-line/series or are arranged in parallel. In some embodiments, the biogas production unit for converting waste to a biogas stream is an anaerobic digester or a landfill. In some embodiments of the systems described herein, the waste is agricultural waste. In some embodiments, the waste is municipal waste, including as examples sewage and landfill waste. In some embodiments, the municipal waste comprises wastewater or leachate.

In a sixth aspect described herein are methods, devices and systems comprising a wet scrubbing system comprising a first absorber that is operatively connected to: (a) a biogas production unit that converts waste to a biogas stream and a liquid waste stream, comprising a biogas stream outlet and a liquid waste stream outlet that connects to a liquid waste stream inlet of at least the first absorber; and (b) a combustion engine that utilizes the biogas stream to produce energy and an engine exhaust stream containing NOx and comprising a biogas stream inlet and an engine exhaust stream outlet connected to the engine exhaust inlet of the first absorber; provided that a second absorber is displaced within the system in-line or parallel with the first absorber.

In a seventh aspect, provided are methods, platforms, devices and systems comprising a multistage wet scrubbing system comprising a liquid waste stream inlet, an engine exhaust inlet, a quencher, cooler and an absorber, provided that the system is connected to: (a) a biogas production unit that converts waste to a biogas stream and a liquid waste stream; and (b) a combustion engine that utilizes the biogas stream to produce energy and an engine exhaust stream containing NOx and comprising a biogas stream inlet and an engine exhaust stream outlet connected to the engine exhaust inlet of an absorber; further provided that a catalyst is contacted with the engine exhaust. In some embodiments, provided is a metal catalyst or zeolite catalyst. In some embodiments, the engine exhaust is cooled to less than about 800° F.

In one aspect described herein is a system comprising: a reactor comprising: a first absorber comprising a first liquid waste stream inlet, a first exhaust inlet, and a first exhaust outlet; and a second absorber comprising a second liquid waste stream inlet, a second exhaust inlet, and a second exhaust outlet; a biogas production unit constructed so as to convert waste to a biogas stream and a liquid waste stream and comprising a biogas stream outlet and a liquid waste stream outlet, the liquid waste stream outlet operatively connected to the first liquid waste stream inlet of the first absorber and operatively connected to the second liquid waste stream inlet of the second absorber; and a device constructed to utilize the biogas stream to produce energy and an exhaust comprising a gas, the device comprising a biogas stream inlet operatively connected to the biogas stream outlet of the biogas production unit and a device exhaust outlet, provided that the device exhaust outlet is operatively connected to the first exhaust inlet of the first absorber, and the first exhaust outlet of the first absorber is operatively connected to the second exhaust inlet of the second absorber, or provided that the device exhaust outlet is operatively connected to the first exhaust inlet of the first absorber and the second exhaust inlet of the second absorber; provided that the reactor is constructed so as to treat exhaust so as to decrease or reduce emissions or pollutants by contacting the exhaust with the liquid waste stream from the biogas production unit.

In some embodiments, the exhaust comprises a gas comprising one or more of NOx, SOx, or COx. In some embodiments, the treated exhaust contains a NOx amount less than or equal to about 0.5 ppm, about 1 ppm, about 2 ppm, about 3 ppm, about 4 ppm, about 5 ppm, about 6 ppm, about 7 ppm, about 8 ppm, about 9 ppm, or about 10 ppm.

In some embodiments, the reactor further comprises a third absorber operatively connected to the first absorber, the second absorber, or the device, the third absorber comprising a third liquid waste stream inlet, a third exhaust inlet, and a third exhaust outlet. In some embodiments the reactor further comprises a fourth absorber operatively connected to the first absorber, the second absorber, the third absorber, or the device, the fourth absorber comprising a fourth liquid waste stream inlet, a fourth exhaust inlet, and a fourth exhaust outlet. In some embodiments the first absorber, the second absorber and the third absorber are aligned in series, in parallel or in a combination of in series and in parallel. In some embodiments, the first absorber, the second absorber the third absorber and the fourth absorber are aligned in series, in parallel or in a combination of in series and in parallel.

In some embodiments the device is an internal combustion engine constructed so as to utilize the biogas stream to produce energy. In some embodiments the device comprises one or more of a biogas engine, an internal combustion engine, a gas fired boiler, a turbine, or a microturbine, the device constructed so as to utilize the biogas stream to produce energy. In some embodiments, the device further comprises a natural gas stream inlet, provided that the device is constructed so that the natural gas stream and the biogas stream are utilized to produce energy. In some embodiments, the device is an internal combustion engine constructed so as to utilize the biogas stream and the natural gas stream to produce energy. In some embodiments, the device comprises one or more of a biogas engine, an internal combustion engine, a gas fired boiler, a turbine, or a microturbine, the device constructed so as to utilize the biogas stream and the natural gas stream to produce energy.

In some embodiments the system further comprises a quencher that is operatively connected to the exhaust outlet of the device. In some embodiments, the system further comprises a cooler that is operatively connected to the quencher. In some embodiments, SOx is effectively removed from the exhaust by cooling the exhaust to a temperature that is less than or equal to about 90° F., about 100° F., about 110° F., about 120° F., about 130° F., about 140° F., or about 150° F. In some embodiments the exhaust is cooled to a temperature that is less than or equal to about 90° F., about 100° F., about 110° F., about 120° F., about 130° F., about 140° F., or about 150° F.

In some embodiments, the biogas production unit for converting waste to a biogas stream is an anaerobic digester or a landfill. In some embodiments, the waste is one or more of agricultural waste and municipal waste. In some embodiments, the municipal waste comprises one or more of wastewater, food waste, and food by-products.

In some embodiments, the first absorber comprises a first nitrate reducing bacterium, the first nitrate reducing bacterium comprising Thiobacillus denitrificans, Micrococcus denitrificans Paracoccus denitrificans or Pseudomonas. In some embodiments, the second absorber comprises a second nitrate reducing bacterium, the second nitrate reducing bacterium comprising Thiobacillus denitrificans, Micrococcus denitrificans Paracoccus denitrificans or Pseudomonas. In some embodiments, the first absorber comprises a first nitrate reducing bacterium, the first nitrate reducing bacterium comprising Thiobacillus denitrificans, Micrococcus denitrificans Paracoccus denitrificans or Pseudomonas, and the second absorber comprises a second nitrate reducing bacterium, the second nitrate reducing bacterium comprising Thiobacillus denitrificans, Micrococcus denitrificans Paracoccus denitrificans or Pseudomonas, provided that the first nitrate reducing bacterium and the second nitrate reducing bacterium are the same or different. In some embodiments, the first absorber comprises a first sulfate-reducing bacterium. In some embodiments, the second absorber comprises a second sulfate-reducing bacterium.

In some embodiments, the system further comprises a first fan. In some embodiments, the system further comprises a first fan, provided that the first fan is operatively connected to the quencher, the first absorber, the second absorber, or a combination thereof. In some embodiments, the system further comprises a first fan, a quencher and a cooler, provided that the first fan is operatively connected to one or more of the quencher, the cooler, the first absorber, or the second absorber. In some embodiments, the system further comprises a first fan and a second fan, provided that the first fan is operatively connected to the first absorber and the second fan is operatively connected to the second absorber. In some embodiments, the system further comprises a first fan, a second fan and a third fan, provided that the first fan is operatively connected to the first absorber, the second fan is operatively connected to the second absorber and the third fan is operatively connected to the third absorber. In some embodiments, the system further comprises a first fan, a second fan, a third fan, and a fourth fan provided that the first fan is operatively connected to the first absorber, the second fan is operatively connected to the second absorber, the third fan is operatively connected to the third absorber and the fourth fan is operatively connected to the fourth absorber.

In some embodiments, the system further comprises a first oxidant source comprising one or more of hydrogen peroxide, ozone, ClO₂, or a photocatalytic oxidation source. In some embodiments, the system further comprises a first oxidant source comprising one or more of hydrogen peroxide, ozone, ClO₂, and a photocatalytic oxidation source and a second oxidant source comprising one or more of hydrogen peroxide, ozone, ClO₂, or a photocatalytic oxidation source, provided that the first oxidant source is operatively connected to the first absorber and the second oxidant source is operatively connected to the second absorber. In some embodiments, the system further comprises a first oxidant source comprising one or more of hydrogen peroxide, ozone, ClO₂, or a photocatalytic oxidation source, a second oxidant source comprising one or more of hydrogen peroxide, ozone, ClO₂, or a photocatalytic oxidation source, and a third oxidant source comprising one or more of hydrogen peroxide, ozone, ClO₂, or a photocatalytic oxidation source, provided that the first oxidant source is operatively connected to the first absorber, the second oxidant source is operatively connected to the second absorber, and the third oxidant source is operatively connected to the third absorber. In some embodiments, the system further comprises a first oxidant source comprising one or more of hydrogen peroxide, ozone, ClO₂, or a photocatalytic oxidation source, a second oxidant source comprising one or more of hydrogen peroxide, ozone, ClO₂, or a photocatalytic oxidation source, a third oxidant source comprising one or more of hydrogen peroxide, ozone, ClO₂, or a photocatalytic oxidation source, and a fourth oxidant source comprising one or more of hydrogen peroxide, ozone, ClO₂, or a photocatalytic oxidation source, provided that the first oxidant source is operatively connected to the first absorber, the second oxidant source is operatively connected to the second absorber, the third oxidant source is operatively connected to the third absorber, and the fourth oxidant source is operatively connected to the fourth absorber. In some embodiments, the first oxidant source is operatively connected to the system so as to access the exhaust. In some embodiments, the first oxidant source provides a single point, continuous infusion of oxidant to the exhaust.

In some embodiments, the reactor further comprises a mist eliminator, the mist eliminator operatively connected to the reactor so as to access the exhaust. In some embodiments, the mist eliminator comprises a polypropylene mesh material, a polypropylene mesh pad, SS-304, 304L, 316, 316L, 430, Monel, Nickel, Copper, P.T.F.E. (Teflon), H.D.P.E., P.P., alloys, drawn plastic, and extruded plastic.

In some embodiments, the system further comprises a first oxidant injection site. In some embodiments, the first oxidant injection site is operatively connected to the system so as to access to the exhaust. In some embodiments, less than a stoichiometric mole of oxidant compared to gas is provided to the system. In some embodiments, the system further comprises an oxidative catalyst. In some embodiments, the oxidative catalyst oxidizes CO to CO2, oxidizes NO to NO₂, or oxidizes both NO to NO₂ and CO to CO₂.

In one aspect described herein is a reactor comprising: a first absorber comprising a first liquid waste stream inlet, a first exhaust inlet, a first exhaust outlet; and a second absorber comprising a second liquid waste stream inlet, a second exhaust inlet, and a second exhaust outlet, the reactor operatively connected to: a biogas production unit constructed so as to convert waste to a biogas stream and a liquid waste stream and comprising a biogas stream outlet and a liquid waste stream outlet, the liquid waste stream outlet operatively connected to the first liquid waste stream inlet of the first absorber and operatively connected to the second liquid waste stream inlet of the second absorber; and a device constructed to utilize the biogas stream to produce energy and an exhaust comprising a gas, the device comprising a biogas stream inlet operatively connected to the biogas stream outlet of the biogas production unit and a device exhaust outlet, provided that the device exhaust outlet is operatively connected to the first exhaust inlet of the first absorber, and the first exhaust outlet of the first absorber is operatively connected to the second exhaust inlet of the second absorber, or provided that the device exhaust outlet is operatively connected to the first exhaust inlet of the first absorber and the second exhaust inlet of the second absorber; provided that the reactor is constructed so as to treat exhaust so as to decrease or reduce emissions or pollutants by contacting the exhaust with the liquid waste stream from the biogas production unit.

In some embodiments, the exhaust comprises a gas comprising one or more of NOx, SOx, or COx. In some embodiments, the treated exhaust contains a NOx amount less than or equal to about 0.5 ppm, about 1 ppm, about 2 ppm, about 3 ppm, about 4 ppm, about 5 ppm, about 6 ppm, about 7 ppm, about 8 ppm, about 9 ppm, or about 10 ppm.

In some embodiments, the reactor further comprises a third absorber operatively connected to the first absorber, the second absorber, or the device, the third absorber comprising a third liquid waste stream inlet, a third exhaust inlet, and a third exhaust outlet. In some embodiments the reactor further comprises a fourth absorber operatively connected to the first absorber, the second absorber, the third absorber, or the device, the fourth absorber comprising a fourth liquid waste stream inlet, a fourth exhaust inlet, and a fourth exhaust outlet. In some embodiments the first absorber, the second absorber and the third absorber are aligned in series, in parallel or in a combination of in series and in parallel. In some embodiments, the first absorber, the second absorber the third absorber and the fourth absorber are aligned in series, in parallel or in a combination of in series and in parallel.

In some embodiments the device is an internal combustion engine constructed so as to utilize the biogas stream to produce energy. In some embodiments the device comprises one or more of a biogas engine, an internal combustion engine, a gas fired boiler, a turbine, or a microturbine, the device constructed so as to utilize the biogas stream to produce energy. In some embodiments, the device further comprises a natural gas stream inlet, provided that the device is constructed so that the natural gas stream and the biogas stream are utilized to produce energy. In some embodiments, the device is an internal combustion engine constructed so as to utilize the biogas stream and the natural gas stream to produce energy. In some embodiments, the device comprises one or more of a biogas engine, an internal combustion engine, a gas fired boiler, a turbine, or a microturbine, the device constructed so as to utilize the biogas stream and the natural gas stream to produce energy.

In some embodiments the reactor further comprises a quencher that is operatively connected to the exhaust outlet of the device. In some embodiments, the reactor further comprises a cooler that is operatively connected to the quencher. In some embodiments, SOx is effectively removed from the exhaust by cooling the exhaust to a temperature that is less than or equal to about 90° F., about 100° F., about 110° F., about 120° F., about 130° F., about 140° F., or about 150° F. In some embodiments the exhaust is cooled to a temperature that is less than or equal to about 90° F., about 100° F., about 110° F., about 120° F., about 130° F., about 140° F., or about 150° F.

In some embodiments, the biogas production unit for converting waste to a biogas stream is an anaerobic digester or a landfill. In some embodiments, the waste is one or more of agricultural waste and municipal waste. In some embodiments, the municipal waste comprises one or more of wastewater, food waste, and food by-products.

In some embodiments, the first absorber comprises a first nitrate reducing bacterium, the first nitrate reducing bacterium comprising Thiobacillus denitrificans, Micrococcus denbrificans Paracoccus denbrificans or Pseudomonas. In some embodiments, the second absorber comprises a second nitrate reducing bacterium, the second nitrate reducing bacterium comprising Thiobacillus denitrificans, Micrococcus denitrificans Paracoccus denitrificans or Pseudomonas. In some embodiments, the first absorber comprises a first nitrate reducing bacterium, the first nitrate reducing bacterium comprising Thiobacillus denitrificans, Micrococcus denitrificans Paracoccus denitrificans or Pseudomonas, and the second absorber comprises a second nitrate reducing bacterium, the second nitrate reducing bacterium comprising Thiobacillus denitrificans, Micrococcus denitrificans Paracoccus denitrificans or Pseudomonas, provided that the first nitrate reducing bacterium and the second nitrate reducing bacterium are the same or different. In some embodiments, the first absorber comprises a first sulfate-reducing bacterium. In some embodiments, the second absorber comprises a second sulfate-reducing bacterium.

In some embodiments, the reactor further comprises a first fan. In some embodiments, the reactor further comprises a first fan, provided that the first fan is operatively connected to the quencher, the first absorber, the second absorber, or a combination thereof. In some embodiments, the reactor further comprises a first fan, a quencher and a cooler, provided that the first fan is operatively connected to one or more of the quencher, the cooler, the first absorber, or the second absorber. In some embodiments, the reactor further comprises a first fan and a second fan, provided that the first fan is operatively connected to the first absorber and the second fan is operatively connected to the second absorber. In some embodiments, the reactor further comprises a first fan, a second fan and a third fan, provided that the first fan is operatively connected to the first absorber, the second fan is operatively connected to the second absorber and the third fan is operatively connected to the third absorber. In some embodiments, the reactor further comprises a first fan, a second fan, a third fan, and a fourth fan provided that the first fan is operatively connected to the first absorber, the second fan is operatively connected to the second absorber, the third fan is operatively connected to the third absorber and the fourth fan is operatively connected to the fourth absorber.

In some embodiments, the reactor further comprises a first oxidant source comprising one or more of hydrogen peroxide, ozone, ClO₂, or a photocatalytic oxidation source. In some embodiments, the reactor further comprises a first oxidant source comprising one or more of hydrogen peroxide, ozone, ClO₂, and a photocatalytic oxidation source and a second oxidant source comprising one or more of hydrogen peroxide, ozone, ClO₂, or a photocatalytic oxidation source, provided that the first oxidant source is operatively connected to the first absorber and the second oxidant source is operatively connected to the second absorber. In some embodiments, the reactor further comprises a first oxidant source comprising one or more of hydrogen peroxide, ozone, ClO₂, or a photocatalytic oxidation source, a second oxidant source comprising one or more of hydrogen peroxide, ozone, ClO₂, or a photocatalytic oxidation source, and a third oxidant source comprising one or more of hydrogen peroxide, ozone, ClO₂, or a photocatalytic oxidation source, provided that the first oxidant source is operatively connected to the first absorber, the second oxidant source is operatively connected to the second absorber, and the third oxidant source is operatively connected to the third absorber. In some embodiments, the reactor further comprises a first oxidant source comprising one or more of hydrogen peroxide, ozone, ClO₂, or a photocatalytic oxidation source, a second oxidant source comprising one or more of hydrogen peroxide, ozone, ClO₂, or a photocatalytic oxidation source, a third oxidant source comprising one or more of hydrogen peroxide, ozone, ClO₂, or a photocatalytic oxidation source, and a fourth oxidant source comprising one or more of hydrogen peroxide, ozone, ClO₂, or a photocatalytic oxidation source, provided that the first oxidant source is operatively connected to the first absorber, the second oxidant source is operatively connected to the second absorber, the third oxidant source is operatively connected to the third absorber, and the fourth oxidant source is operatively connected to the fourth absorber. In some embodiments, the first oxidant source is operatively connected to the reactor so as to access the exhaust. In some embodiments, the first oxidant source provides a single point, continuous infusion of oxidant to the exhaust.

In some embodiments, the reactor further comprises a mist eliminator, the mist eliminator operatively connected to the reactor so as to access the exhaust. In some embodiments, the mist eliminator comprises a polypropylene mesh material, a polypropylene mesh pad, SS-304, 304L, 316, 316L, 430, Monel, Nickel, Copper, P.T.F.E. (Teflon), H.D.P.E., P.P., alloys, drawn plastic, and extruded plastic.

In some embodiments, the reactor further comprises a first oxidant injection site. In some embodiments, the first oxidant injection site is operatively connected to the reactor so as to access to the exhaust. In some embodiments, less than a stoichiometric mole of oxidant compared to gas is provided to the reactor. In some embodiments, the reactor further comprises an oxidative catalyst. In some embodiments, the oxidative catalyst oxidizes CO to CO2, oxidizes NO to NO₂, or oxidizes both NO to NO₂ and CO to CO₂.

In one aspect, provided is a method to treat exhaust comprising: providing a system comprising: a reactor comprising: a first absorber comprising a first liquid waste stream inlet, a first exhaust inlet and a first exhaust outlet; and a second absorber comprising a second liquid waste stream inlet, a second exhaust inlet and a second exhaust outlet; a biogas production unit constructed so as to convert waste to a biogas stream and a liquid waste stream and comprising a biogas stream outlet and a liquid waste stream outlet, the liquid waste stream outlet operatively connected to the first liquid waste stream inlet of the first absorber and operatively connected to the second liquid waste stream inlet of the second absorber; and a device constructed to utilize the biogas stream to produce energy and an exhaust comprising a gas, the device comprising a biogas stream inlet operatively connected to the biogas stream outlet of the biogas production unit and a device exhaust outlet, provided that the device exhaust outlet is operatively connected to the first exhaust inlet of the first absorber, and the first exhaust outlet of the first absorber is operatively connected to the second exhaust inlet of the second absorber, or provided that the device exhaust outlet is operatively connected to the first exhaust inlet of the first absorber and the second exhaust inlet of the second absorber; and delivering waste to the biogas production unit; generating exhaust comprising the gas using the device; and contacting the exhaust with the liquid waste stream so as to treat exhaust so as to decrease or reduce emissions or pollutants.

In some embodiments, the exhaust comprises a gas comprising one or more of NOx, SOx, or COx. In some embodiments, the treated exhaust contains a NOx amount less than or equal to about 0.5 ppm, about 1 ppm, about 2 ppm, about 3 ppm, about 4 ppm, about 5 ppm, about 6 ppm, about 7 ppm, about 8 ppm, about 9 ppm, or about 10 ppm.

In some embodiments, the reactor further comprises a third absorber operatively connected to the first absorber, the second absorber, or the device, the third absorber comprising a third liquid waste stream inlet, a third exhaust inlet, and a third exhaust outlet. In some embodiments the reactor further comprises a fourth absorber operatively connected to the first absorber, the second absorber, the third absorber, or the device, the fourth absorber comprising a fourth liquid waste stream inlet, a fourth exhaust inlet, and a fourth exhaust outlet. In some embodiments the first absorber, the second absorber and the third absorber are aligned in series, in parallel or in a combination of in series and in parallel. In some embodiments, the first absorber, the second absorber the third absorber and the fourth absorber are aligned in series, in parallel or in a combination of in series and in parallel.

In some embodiments the device is an internal combustion engine constructed so as to utilize the biogas stream to produce energy. In some embodiments the device comprises one or more of a biogas engine, an internal combustion engine, a gas fired boiler, a turbine, or a microturbine, the device constructed so as to utilize the biogas stream to produce energy. In some embodiments, the device further comprises a natural gas stream inlet, provided that the device is constructed so that the natural gas stream and the biogas stream are utilized to produce energy. In some embodiments, the device is an internal combustion engine constructed so as to utilize the biogas stream and the natural gas stream to produce energy. In some embodiments, the device comprises one or more of a biogas engine, an internal combustion engine, a gas fired boiler, a turbine, or a microturbine, the device constructed so as to utilize the biogas stream and the natural gas stream to produce energy.

In some embodiments the system further comprises a quencher that is operatively connected to the exhaust outlet of the device. In some embodiments, the system further comprises a cooler that is operatively connected to the quencher. In some embodiments, SOx is effectively removed from the exhaust by cooling the exhaust to a temperature that is less than or equal to about 90° F., about 100° F., about 110° F., about 120° F., about 130° F., about 140° F., or about 150° F. In some embodiments the exhaust is cooled to a temperature that is less than or equal to about 90° F., about 100° F., about 110° F., about 120° F., about 130° F., about 140° F., or about 150° F.

In some embodiments, the biogas production unit for converting waste to a biogas stream is an anaerobic digester or a landfill. In some embodiments, the waste is one or more of agricultural waste and municipal waste. In some embodiments, the municipal waste comprises one or more of wastewater, food waste, and food by-products.

In some embodiments, the first absorber comprises a first nitrate reducing bacterium, the first nitrate reducing bacterium comprising Thiobacillus denitrificans, Micrococcus denitrificans Paracoccus denitrificans or Pseudomonas. In some embodiments, the second absorber comprises a second nitrate reducing bacterium, the second nitrate reducing bacterium comprising Thiobacillus denitrificans, Micrococcus denitrificans Paracoccus denitrificans or Pseudomonas. In some embodiments, the first absorber comprises a first nitrate reducing bacterium, the first nitrate reducing bacterium comprising Thiobacillus denitrificans, Micrococcus denitrificans Paracoccus denitrificans or Pseudomonas, and the second absorber comprises a second nitrate reducing bacterium, the second nitrate reducing bacterium comprising Thiobacillus denitrificans, Micrococcus denitrificans Paracoccus denitrificans or Pseudomonas, provided that the first nitrate reducing bacterium and the second nitrate reducing bacterium are the same or different. In some embodiments, the first absorber comprises a first sulfate-reducing bacterium. In some embodiments, the second absorber comprises a second sulfate-reducing bacterium.

In some embodiments, the system further comprises a first fan. In some embodiments, the system further comprises a first fan, provided that the first fan is operatively connected to the quencher, the first absorber, the second absorber, or a combination thereof. In some embodiments, the system further comprises a first fan, a quencher and a cooler, provided that the first fan is operatively connected to one or more of the quencher, the cooler, the first absorber, or the second absorber. In some embodiments, the system further comprises a first fan and a second fan, provided that the first fan is operatively connected to the first absorber and the second fan is operatively connected to the second absorber. In some embodiments, the system further comprises a first fan, a second fan and a third fan, provided that the first fan is operatively connected to the first absorber, the second fan is operatively connected to the second absorber and the third fan is operatively connected to the third absorber. In some embodiments, the system further comprises a first fan, a second fan, a third fan, and a fourth fan provided that the first fan is operatively connected to the first absorber, the second fan is operatively connected to the second absorber, the third fan is operatively connected to the third absorber and the fourth fan is operatively connected to the fourth absorber.

In some embodiments, the system further comprises a first oxidant source comprising one or more of hydrogen peroxide, ozone, ClO₂, or a photocatalytic oxidation source. In some embodiments, the system further comprises a first oxidant source comprising one or more of hydrogen peroxide, ozone, ClO₂, and a photocatalytic oxidation source and a second oxidant source comprising one or more of hydrogen peroxide, ozone, ClO₂, or a photocatalytic oxidation source, provided that the first oxidant source is operatively connected to the first absorber and the second oxidant source is operatively connected to the second absorber. In some embodiments, the system further comprises a first oxidant source comprising one or more of hydrogen peroxide, ozone, ClO₂, or a photocatalytic oxidation source, a second oxidant source comprising one or more of hydrogen peroxide, ozone, ClO₂, or a photocatalytic oxidation source, and a third oxidant source comprising one or more of hydrogen peroxide, ozone, ClO₂, or a photocatalytic oxidation source, provided that the first oxidant source is operatively connected to the first absorber, the second oxidant source is operatively connected to the second absorber, and the third oxidant source is operatively connected to the third absorber. In some embodiments, the system further comprises a first oxidant source comprising one or more of hydrogen peroxide, ozone, ClO₂, or a photocatalytic oxidation source, a second oxidant source comprising one or more of hydrogen peroxide, ozone, ClO₂, or a photocatalytic oxidation source, a third oxidant source comprising one or more of hydrogen peroxide, ozone, ClO₂, or a photocatalytic oxidation source, and a fourth oxidant source comprising one or more of hydrogen peroxide, ozone, ClO₂, or a photocatalytic oxidation source, provided that the first oxidant source is operatively connected to the first absorber, the second oxidant source is operatively connected to the second absorber, the third oxidant source is operatively connected to the third absorber, and the fourth oxidant source is operatively connected to the fourth absorber. In some embodiments, the first oxidant source is operatively connected to the system so as to access the exhaust. In some embodiments, the first oxidant source provides a single point, continuous infusion of oxidant to the exhaust.

In some embodiments, the reactor further comprises a mist eliminator, the mist eliminator operatively connected to the reactor so as to access the exhaust. In some embodiments, the mist eliminator comprises a polypropylene mesh material, a polypropylene mesh pad, SS-304, 304L, 316, 316L, 430, Monel, Nickel, Copper, P.T.F.E. (Teflon), H.D.P.E., P.P., alloys, drawn plastic, and extruded plastic.

In some embodiments, the system further comprises a first oxidant injection site. In some embodiments, the first oxidant injection site is operatively connected to the system so as to access to the exhaust. In some embodiments, less than a stoichiometric mole of oxidant compared to gas is provided to the system. In some embodiments, the system further comprises an oxidative catalyst. In some embodiments, the oxidative catalyst oxidizes CO to CO2, oxidizes NO to NO₂, or oxidizes both NO to NO₂ and CO to CO₂.

### BRIEF DESCRIPTION OF THE DRAWINGS

The novel features of the subject matter described herein are set forth with particularity in the appended claims. A better understanding of the features and advantages of the presently disclosed subject matter will be obtained by reference to the following detailed description that sets forth illustrative embodiments, in which the principles of the subject matter are utilized, and the accompanying drawings.

FIG. 1 is a non-limiting example of the methods, devices and systems provided herein.

FIG. 2 is a non-limiting example of a quencher and a cooler, the quencher and cooler being operatively connected.

FIG. 3 is a non-limiting example of a quencher, a cooler, a first absorber a mist eliminator and a fan, the quencher, cooler first absorber, mist eliminator and fan being operatively connected.

FIG. 4 is a non-limiting example of a quencher, a cooler, a mist eliminator, a first absorber, and a second absorber, the quencher, cooler, mist eliminator, first absorber, and second absorber being operatively connected.

FIG. 5 is a non-limiting example of a third absorber and a fourth absorber, the third and fourth absorber being operatively connected.

FIG. 6 is a non-limiting example of an elevation view of the methods, devices and systems described herein comprising a first absorber, a second absorber, a third absorber, a quencher, a cooler, and a fan, the first absorber, the second absorber, the third absorber, the quencher, the cooler, and the fan being operatively connected.

FIG. 7A and FIG. 7B are a non-limiting examples of the methods, devices and systems described herein comprising a quencher, a cooler a mist eliminator, a first absorber, a second absorber a third absorber and a fan. As shown in FIG. 7B, an oxidant is introduced into the exhaust stream prior to introduction of the exhaust into an absorber.

FIG. 8 is a non-limiting example of an elevation view of the methods, devices and systems described herein comprising a first absorber, a second absorber, a third absorber, a fourth absorber a quencher, a cooler, and a fan, the first absorber, the second absorber, the third absorber, the fourth absorber, the quencher, the cooler, and the fan being operatively connected.

FIG. 9A and FIG. 9B are non-limiting examples of the methods, devices and systems described herein comprising a quencher, a cooler, a mist eliminator, a first absorber, a second absorber, a third absorber, a fourth absorber, and a fan, all of which are operatively connected. Further depicted in FIG. 9A is the injection of an oxidant.

FIG. 10A is a non-limiting example of a sump pump and a non-limiting example of a supply pump is shown in FIG. 10B.

FIG. 11 is a non-limiting example of an elevation view depicting implementation of the platforms, methods, devices and systems in a California dairy.

FIG. 12A, FIG. 12B, and FIG. 12C are non-limiting examples of four-stage reactors aligned in series and in parallel, each stage comprising an absorber. In some embodiments, a fan is operatively connected to one or both four stage reactors.

FIG. 13A is a non-limiting example of a first absorber and a second absorber aligned in series, and FIG. 13B is a non-limiting example of a first absorber and a second absorber aligned in series. In some embodiments, FIG. 13A depicts a front view of the absorbers and FIG. 13B depicts a rear view of the absorbers.

FIG. 14A is a non-limiting example of a first absorber, a second absorber, a third absorber, and a fourth absorber aligned in series, and FIG. 14B is a non-limiting example of a first absorber, a second absorber, a third absorber, and a fourth absorber aligned in series. In some embodiments, FIG. 14A depicts a top view of the absorbers and FIG. 14B depicts a front view of the absorbers.

FIG. 15 is a non-limiting example of an elevation view of a device room, a tank, a dewatering building and a system area comprising a sump, one or more absorbers, a sump pump an oxidant source, and a control room.

FIG. 16 is a non-limiting example of an elevation view of a system comprising a sump area, a first reactor, a second reactor, a first absorber, a second absorber, a quencher, a cooler, a fan, an oxygen source, and a control panel room.

FIG. 17 is a non-limiting example of a quencher and a cooler, the quencher and cooler being operatively connected.

FIG. 18 is a non-limiting example of an elevation view depicting implementation of the platforms, methods, devices and systems in a wastewater treatment facility.

### DETAILED DESCRIPTION OF THE INVENTION

The embodiments of the subject matter described herein illustrate technologies that reduce harmful emissions produced from engines such as combustion engines.

Anaerobic digestion, when utilized at wastewater treatment plants, diaries, and landfills, is the source of biogas, which is used as a fuel source by an appropriate engine. EPA and state regional air boards have stringent emission regulations and standards governing the exhaust from biogas combustion. The exhaust from biogas combustion contains NOx, which is known to be a pre-cursor to ground level ozone, which causes an array of health problems including asthma.

The United States Environmental Protection Agency (US EPA) has designated many regions as “Ground Level Ozone Non-Attainment,” meaning the air quality is poor, negatively impacting those living and working in the area. Ground-level ozone is not a pollutant emitted by combustion engines directly into the air, but is created through photochemical reactions between oxides of nitrogen, volatile organic compounds and sunlight. NOx is emitted by internal combustion engines, and VOCs are generated from gasoline vapors, and chemical solvents. Together, NOx and VOCs combine in the atmosphere creating damaging ground level ozone that is a major health and economic concern.

The NOx reduction rate or efficiency for Selective Catalytic Reduction (SCR) systems is good; however, SCR systems do not work well in high sulfur and siloxane environments. Oxidized Sulfur and siloxane compounds in the exhaust stream precipitate out over the catalytic bed, coating or “poisoning” the catalyst, destroying the system's NOx reduction capability. Thus, SCR systems require highly effective and expensive H₂S and siloxane removal systems to treat the biogas stream prior to combustion. Additionally, SCR has a variable operating cost that includes continual urea purchases, catalyst replacement, in addition to maintenance on and variable costs associated with the H₂S and siloxane reduction component.

SCR systems are capable of reducing NOx to the 9-11 ppm level in an exhaust stream from a lean burn biogas engine. However, SCR is not capable of meeting the California Air Resource Board (CARB) standard of 2-3 ppm and other federal and regional threshold levels. Furthermore, traditional SCR systems do not reduce SOx emissions.

Selective Non-Catalytic Reduction (or SNCR) does not require a catalyst, and thus there is no need for H₂S and siloxane reduction prior to combustion. The chemical reaction in SNCR occurs only when the exhaust stream is heated up to 1450° F. The energy required to heat an exhaust stream from approximately 700° F. to 1450° F. makes SNCR prohibitively expensive for most biogas projects. Here too, traditional SNCR systems do not reduce SOx emissions.

Biogas projects in California and other regions have had difficulty obtaining air permits for operation due to NOx levels in the exhaust stream of the biogas engine. Existing NOx reduction solutions, (traditional uses of SCR and SNCR) are costly, and are not capable of meeting the CARB standard of 2-3 ppm. Furthermore, sulfur and siloxane levels in biogas hinder catalytic processes that reduce NOx emissions.

The subject matter described herein enables a biogas project to use a low cost and highly efficient internal combustion engine to produce both electrical and thermal energy in combination with additional optional features and other technologies, including but not limited to fuel cells, micro-turbines, upgrading biogas for the pipeline, or gasification. A feature of the subject matter provided herein reduces NOx and SOx emissions from biogas combustion by utilizing the liquid waste stream from the anaerobic digester in contact with the biogas engine exhaust.

One feature of the subject matter described herein is a wet scrubbing, non-catalytic process used at low temperatures to reduce the regulated pollutants NOx and SOx.

The embodiments described herein have demonstrated efficacy at a dairy based biogas project in California. SOx emissions were reduced to less than 0.1 ppm, and NOx emissions were reduced to CARB levels of 2-3 ppm, which is significantly below the current BACT technology, urea injected SCR (which has proven capabilities of reducing NOx to 9-11 ppm). Additionally, the subject matter described herein has flexibility to keep up with increasingly stringent regulations for reducing NOx emissions through retroactive incorporation of additional absorption stages. Existing NOx reduction solutions lack this flexibility. Furthermore, the subject matter described herein has proven to be highly sulfur tolerant, negating the need for expensive H₂S removal technology to treat the biogas stream prior to combustion. However, in some embodiments, if H₂S and siloxane removal systems are incorporated to the subject matter described herein; for example, a catalytic stage that oxidizes approximately 50% of the NO to NO₂ and approximately 95% of the CO to CO₂ is used in conjunction with a wet scrubbing system described herein.

In some embodiments of the systems, methods, devices described herein, operatively connected comprises a direct connection or an indirect connection. In some embodiments, a first component and a second component of a system or method or device are directly connected when there are no additional components between the first component and the second component. In some embodiments, a first component and a second component of a system, method or device are indirectly connected when there are additional components between the first component and the second component. In some embodiments, a first component and a second component of a system, method or device are operatively connected by a duct, pipe, tube, or other means of delivering a substance from one location to another. In some embodiments, a first component and a second component of a system, method or device are operatively connected by a duct, pipe, tube, or other means of delivering a substance from one location to another such that the first and/or the second component is injected, infused, and/or included a duct, pipe, tube, or other means of delivering a substance from one location to another.

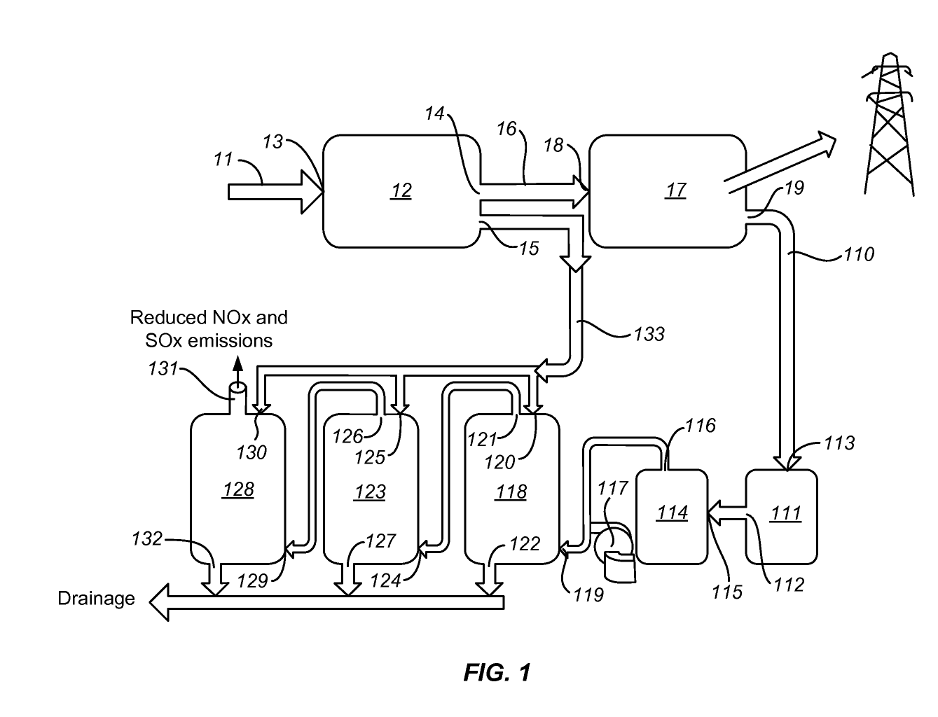

A non-limiting example of the methods, devices and systems described herein as depicted in FIG. 1 comprises a first absorber 118 comprising a first liquid waste stream inlet 120 a first exhaust inlet 119, and a first exhaust outlet 121; a biogas production unit 12 constructed so as to convert waste to a biogas stream 16 and a liquid waste stream 133 and comprising a liquid waste stream inlet 13, a biogas stream outlet 14 and a liquid waste stream outlet 15 operatively connected to the first liquid waste stream inlet of the first absorber; and a device 17 constructed to utilize the biogas stream to produce energy and an exhaust 110 comprising a gas, the device comprising a biogas stream inlet 18 operatively connected to the biogas stream outlet of the biogas production unit and an exhaust outlet 19 operatively connected to the first exhaust inlet of the first absorber, provided that the reactor is constructed so as to treat exhaust so as to decrease or reduce emissions or pollutants by contacting the exhaust with the liquid waste stream from the biogas production unit. In some embodiments, the methods, devices and systems comprise a second absorber 123 comprising a second liquid waste stream inlet 125, a second exhaust inlet 124, and a second exhaust outlet 126, provided that the liquid waste stream outlet of the biogas production unit is operatively connected to the first liquid waste stream inlet of the first absorber and the second liquid waste stream inlet of the second absorber, and the first exhaust outlet of the first absorber is operatively connected to the second exhaust inlet of the second absorber. In some embodiments, the methods, devices and systems comprise a second absorber 123 comprising a second liquid waste stream inlet 125, a second exhaust inlet 124, and a second exhaust outlet 126 and a third absorber 128 comprising a third liquid waste stream inlet 130, a third exhaust inlet 129, and a third exhaust outlet 131, provided that the liquid waste stream outlet of the biogas production unit is operatively connected to the first liquid waste stream inlet of the first absorber, the second liquid waste stream inlet of the second absorber and the third liquid waste stream inlet of the third absorber, the first exhaust outlet of the first absorber is operatively connected to the second exhaust inlet of the second absorber and the second exhaust outlet of the second absorber is operatively connected to the third exhaust inlet of the third absorber. In some embodiments, the first absorber comprises a first liquid waste stream outlet 122. In some embodiments, the second absorber comprises a second liquid waste stream outlet 127. In some embodiments, the third absorber comprises a third liquid waste stream outlet 132. In some embodiments, the methods, devices and systems described herein further comprise a quencher 111 comprising a quencher exhaust inlet 113 operatively connected to the device exhaust outlet 19 and a quencher exhaust outlet 112. In some embodiments, the methods, devices and systems described herein further comprise a cooler 114 comprising a cooler exhaust inlet 115 and a cooler exhaust outlet 116. In some embodiments, the quencher exhaust outlet is operatively connected to the cooler exhaust inlet. In some embodiments, the cooler exhaust outlet is operatively connected to the first absorber, optionally through the first exhaust inlet. In some embodiments, the methods, devices and systems described herein further comprise a fan 117. In some embodiments, the fan is operatively connected to the first absorber. In some embodiments, the methods, devices and systems described herein further comprises a waste stream 11 delivered to the biogas production unit.

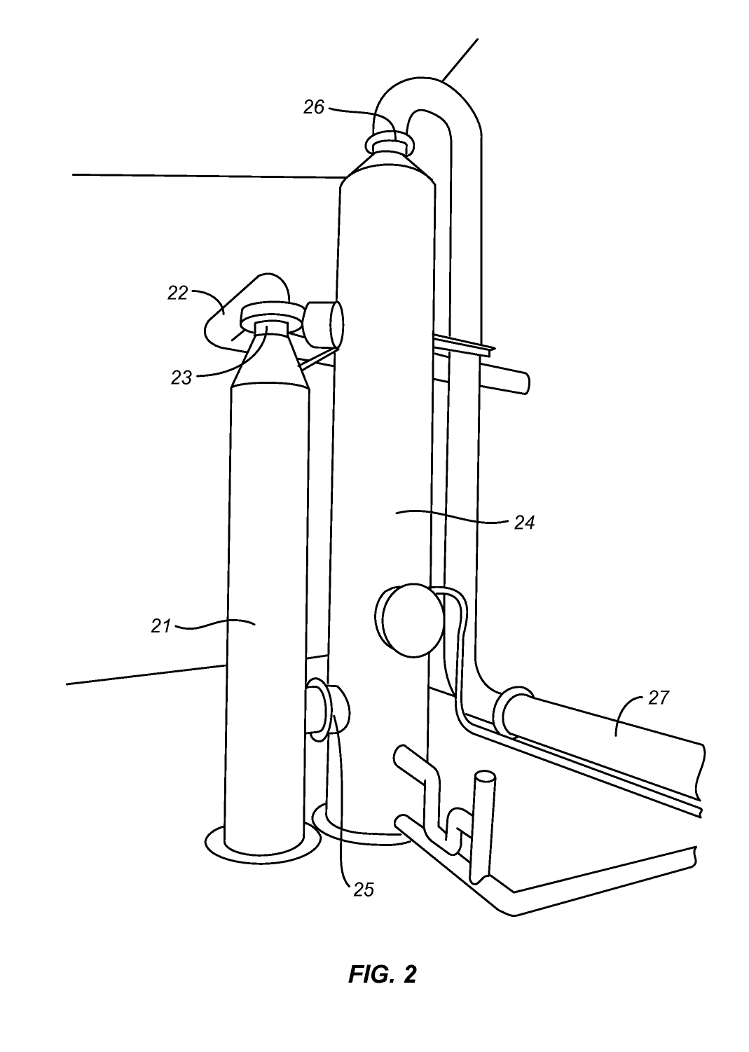

A non-limiting example of the methods, devices and systems described herein as depicted in FIG. 2 comprises a quencher 21 and a cooler 24, the quencher and cooler being operatively connected. In some embodiments the quencher 21 comprises a quencher exhaust inlet 23 operatively connected 22 to the device exhaust outlet so as to deliver exhaust to the quencher. In some embodiments the cooler 24 comprises a cooler exhaust inlet 25 and a cooler exhaust outlet 26. In some embodiments, the quencher exhaust outlet is operatively connected to the cooler exhaust inlet. In some embodiments, the cooler is operatively connected 27 to a first absorber.

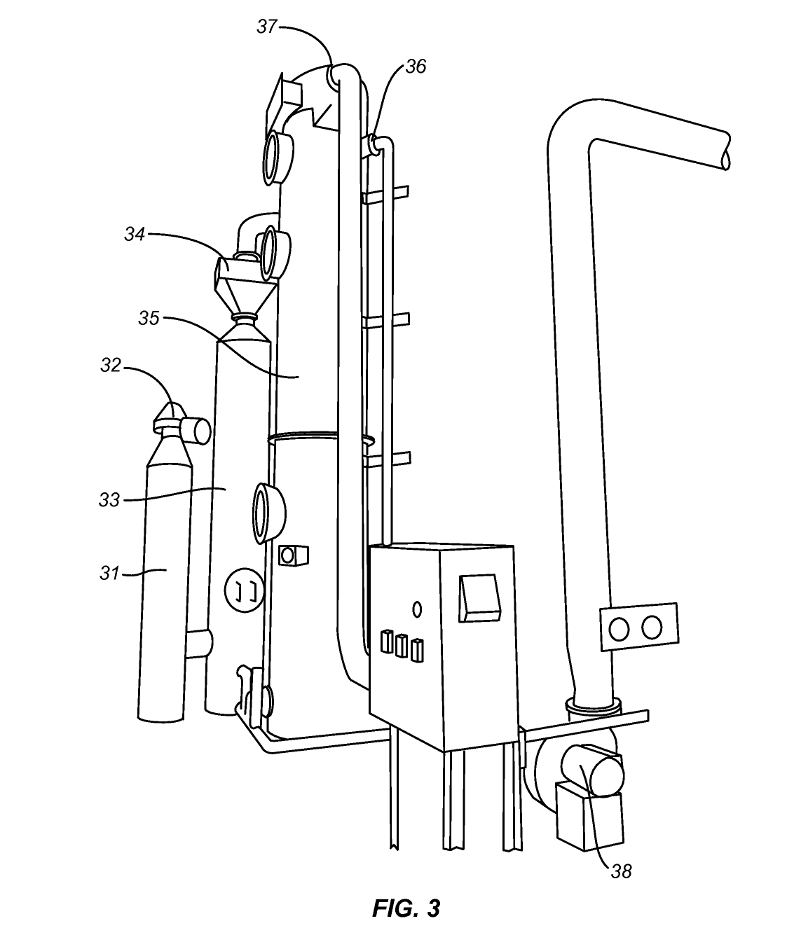

A non-limiting example of the methods, devices and systems described herein as depicted in FIG. 3 comprises a quencher 31, a cooler 33, a first absorber 35 and a fan 38, the quencher, cooler first absorber and fan being operatively connected. In some embodiments the quencher 31 comprises a quencher exhaust inlet 32. In some embodiments the mist eliminator 34 is operatively connected to the cooler and the first absorber. In some embodiments, the first absorber comprises a first exhaust outlet 37 and a first liquid waste stream inlet 36.

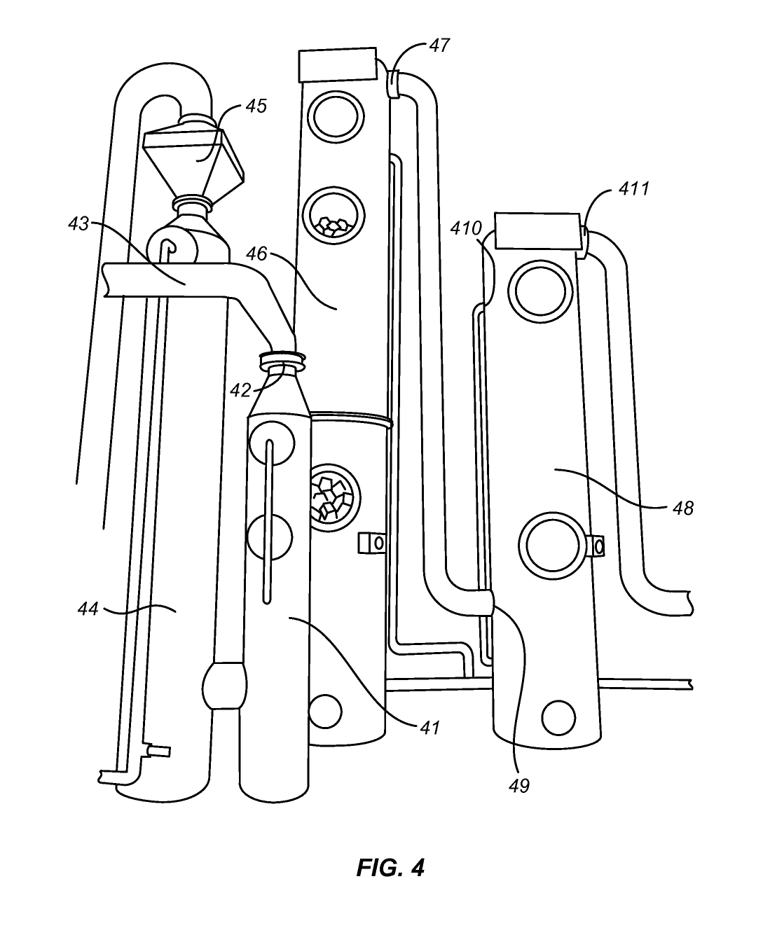

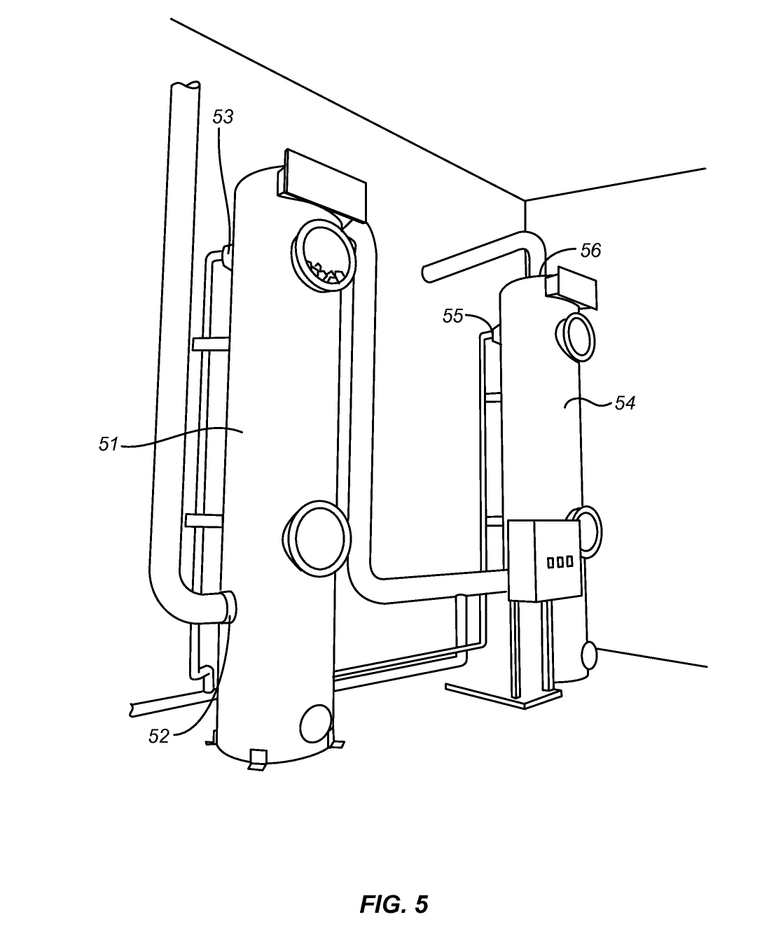

A non-limiting example of the methods, devices and systems described herein as depicted in FIG. 4 comprises a quencher 41, a cooler 44, a mist eliminator 45, a first absorber 46, and a second absorber 48, the quencher, cooler, mist eliminator, first absorber, and second absorber being operatively connected. In some embodiments the quencher 41 comprises a quencher exhaust inlet 42 operatively connected 43 to the device exhaust outlet so as to deliver exhaust to the quencher. In some embodiments the mist eliminator 45 is operatively connected to the cooler and the first absorber. In some embodiments the first absorber 46 comprises a first exhaust outlet 47. In some embodiments, the second absorber 48 comprises a second exhaust inlet 49 operatively connected to the first exhaust outlet 47, a second exhaust outlet 411, and a second liquid waste stream inlet 410.

A non-limiting example of the methods, devices and systems described herein as depicted in FIG. 5 comprises a third absorber 51 and a fourth absorber 54, the third and fourth absorber being operatively connected. In some embodiments the third absorber 51 comprises a third liquid waste stream inlet 53. In some embodiments the fourth absorber 54 comprises a fourth liquid waste stream inlet 55 and a fourth exhaust outlet 56.

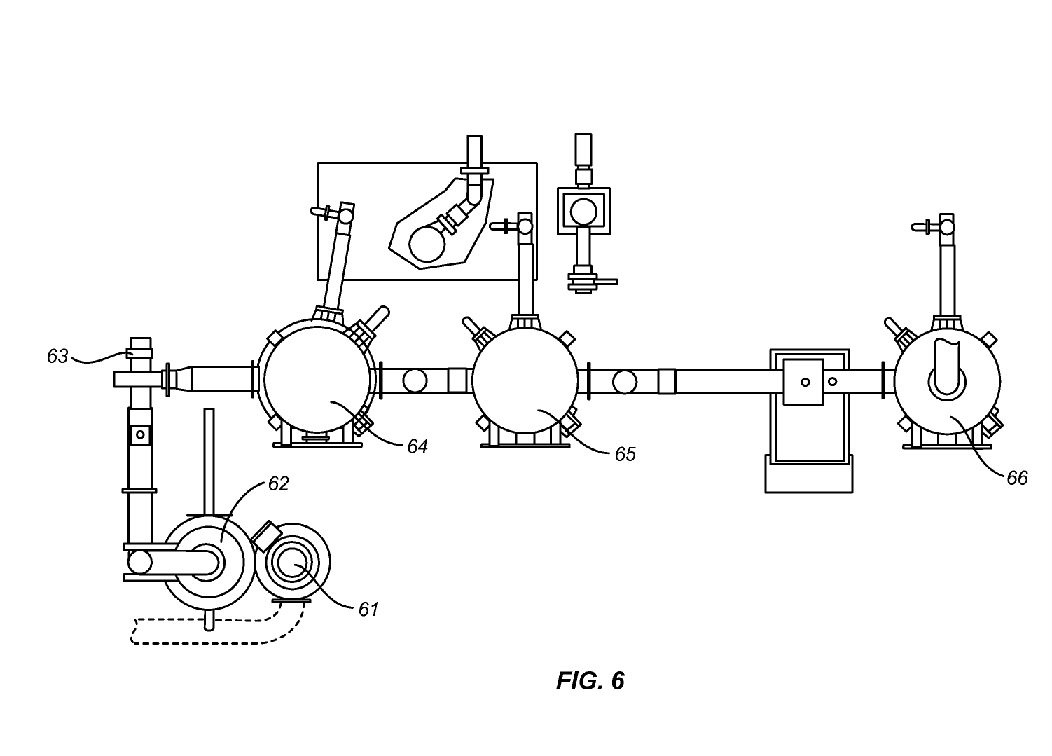

A non-limiting example of the methods, devices and systems described herein as depicted in an elevation view in FIG. 6 comprises a first absorber 64, a second absorber 65, a third absorber 66, a quencher 61, a cooler 62, and a fan 63, the first absorber, the second absorber, the third absorber, the quencher, the cooler, and the fan being operatively connected.

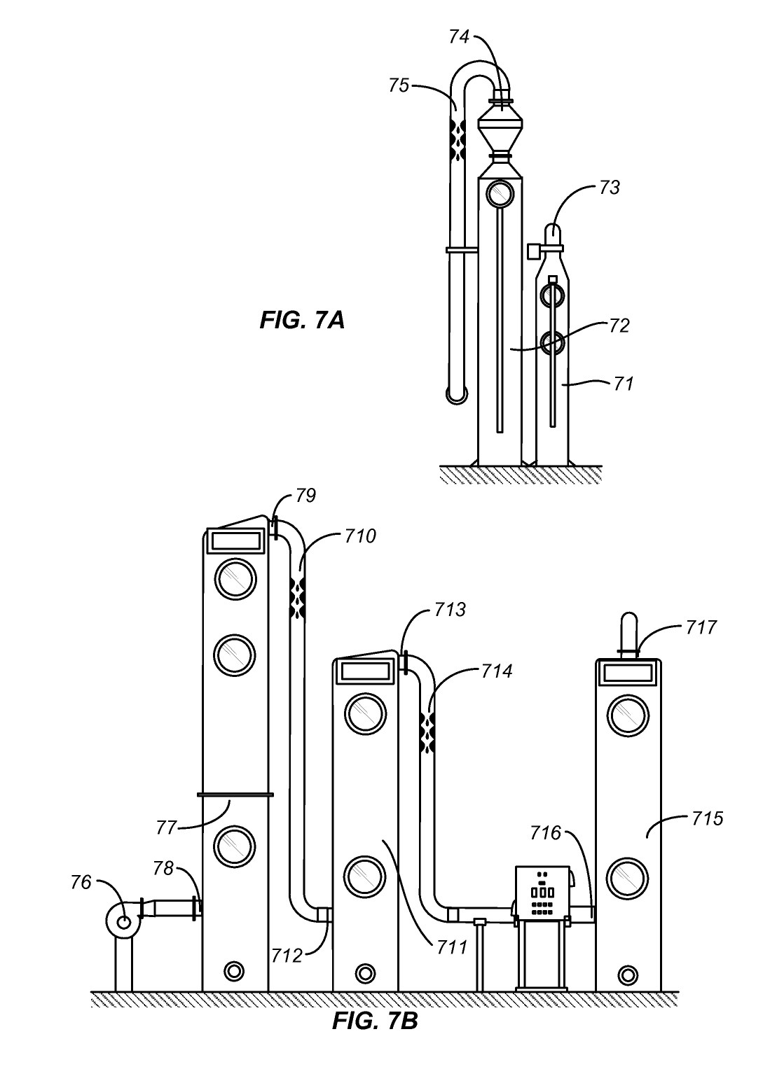

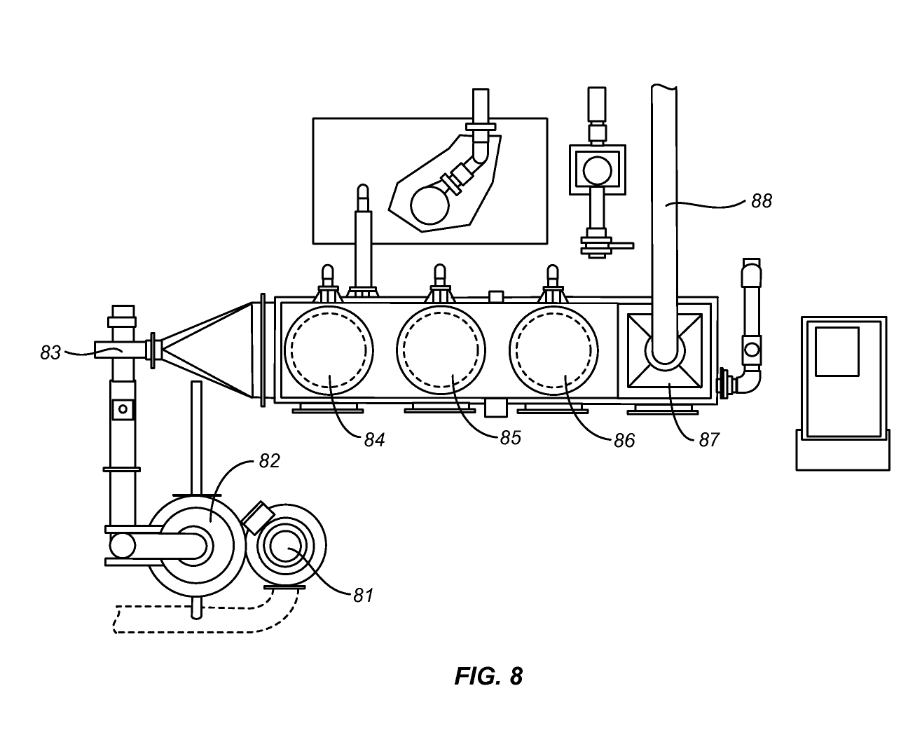

A non-limiting example of the methods, devices and systems described herein as depicted in FIG. 7 comprises a quencher 71, a cooler 72 a mist eliminator 74, a first absorber 77, a second absorber 711, a third absorber 715, and a fan 76. In some embodiments, the quencher 71 comprises a quencher exhaust inlet 73. In some embodiments, the mist eliminator 74 is operatively connected to the cooler 72 and the first absorber 77. In some embodiments a first oxidant inlet 75 is included such that an oxidant is introduced into the exhaust stream after the mist eliminator 74 and prior to introduction of the exhaust into an absorber. In some embodiments, the methods, devices and systems described herein further comprise a fan 76. In some embodiments, the fan is operatively connected to the first absorber and/or the cooler. In some embodiments, the first absorber 77 comprises a first exhaust inlet 78 and a first exhaust outlet 79 operatively connected to the second exhaust inlet of the second absorber 712. In some embodiments a second oxidant inlet 710 is included such that an oxidant is introduced into the exhaust stream prior to introduction of the exhaust into the second absorber. In some embodiments, the second absorber 711 comprises a second exhaust inlet 712 and a second exhaust outlet 713 operatively connected to the third exhaust inlet 716 of the third absorber 715. In some embodiments a third oxidant inlet 714 is included such that an oxidant is introduced into the exhaust stream prior to introduction of the exhaust into the third absorber. In some embodiments, the third absorber 715 comprises a third exhaust inlet 716 and a third exhaust outlet 717.

A non-limiting example of the methods, devices and systems described herein as depicted in an elevation view in FIG. 8 comprises a first absorber 84, a second absorber 85, a third absorber 86, a fourth absorber 87, a quencher 81, a cooler 82, and a fan 83, the first absorber, the second absorber, the third absorber, the fourth absorber, the quencher, the cooler, and the fan being operatively connected. In some embodiments, the exhaust exits the fourth absorber via an exhaust outlet duct 88.

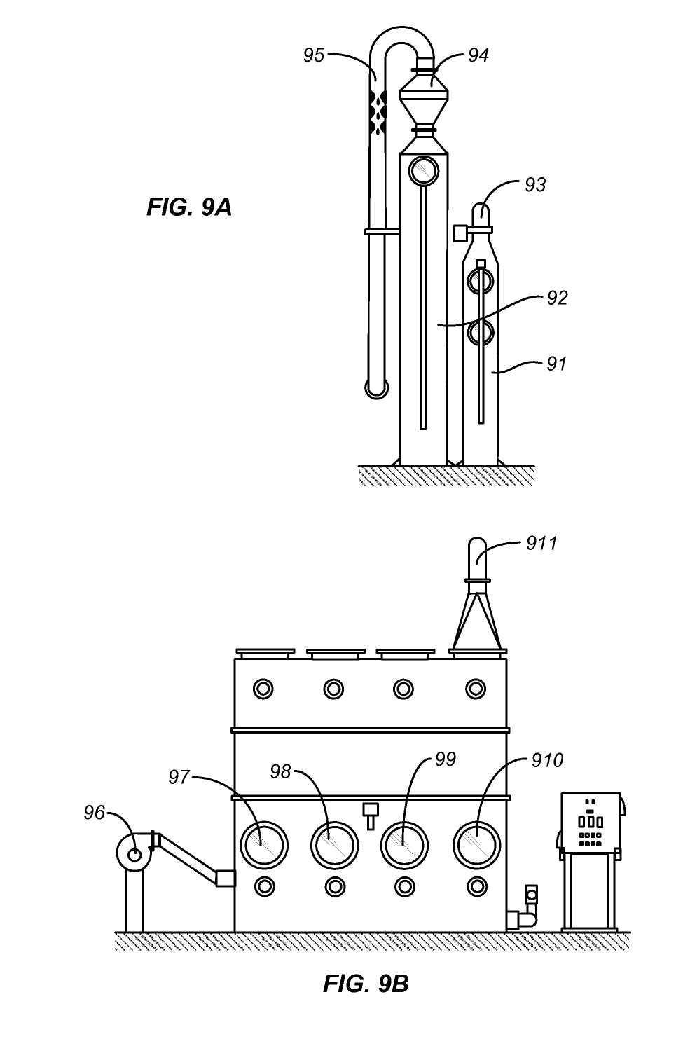

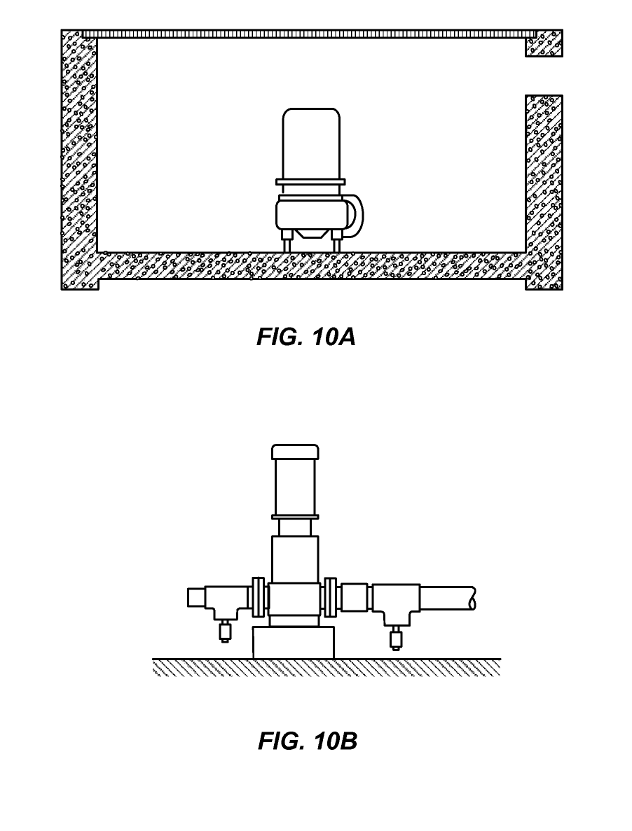

A non-limiting example of the methods, devices and systems described herein as depicted in FIG. 9 comprises a quencher 91, a cooler 92 a mist eliminator 94, a first absorber 97, a second absorber 98, a third absorber 99, a fourth absorber 910, and a fan 96. In some embodiments, the quencher 91 comprises a quencher exhaust inlet 93. In some embodiments, the mist eliminator 94 is operatively connected to the cooler 92 and the first absorber 97. In some embodiments a first oxidant inlet 95 is included such that an oxidant is introduced into the exhaust stream prior to introduction of the exhaust into an absorber. In some embodiments, the methods, devices and systems described herein further comprise a fan 96. In some embodiments, the fan 96 is operatively connected to the first absorber 97 and/or the cooler 92. In some embodiments the fourth absorber comprises an exhaust outlet 911. In some embodiments, the four absorbers are contained in a box. In some embodiments, the fourth exhaust outlet 911 is operatively connected to the box comprising the four absorbers.

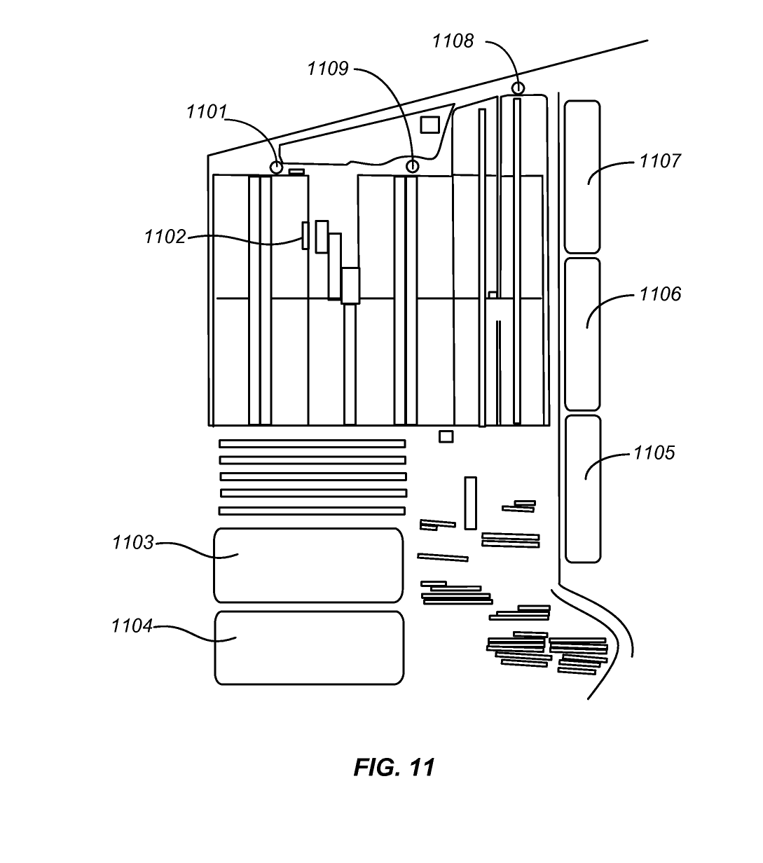

A non-limiting example showing implementation of the platforms, methods, devices and systems in a California dairy is depicted in FIG. 11. In some embodiments, the implementation of the platforms, methods, devices and systems comprises one or more wastewater tanks 1101, 1108 and 1109, a biogas engine 1102, wastewater 1103, an anaerobic digester 1104, and freshwater lagoons 1105, 1106 and 1107.

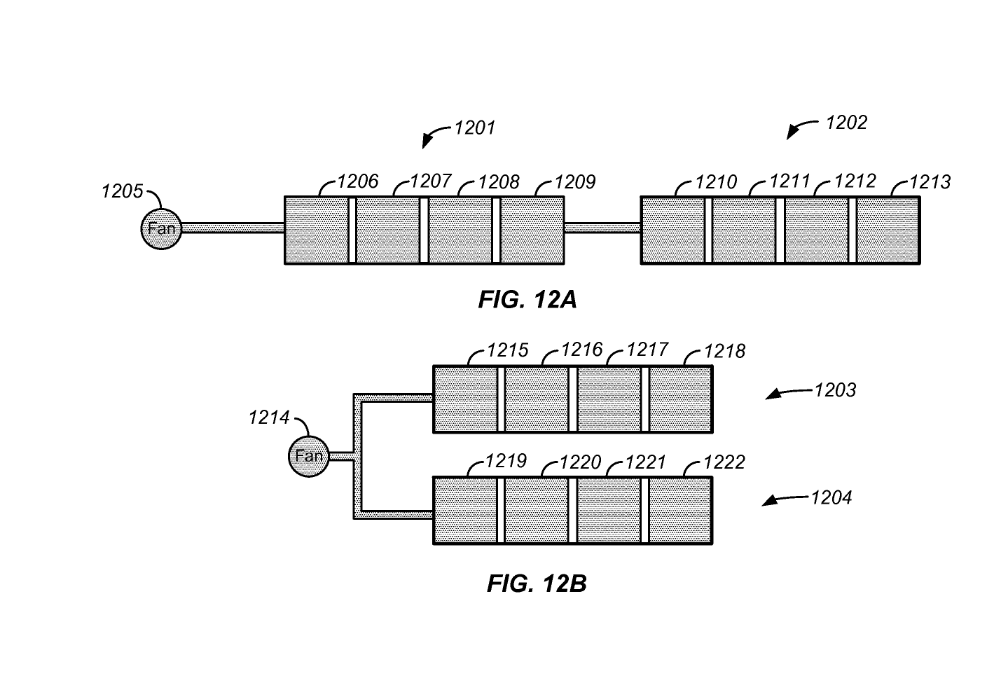

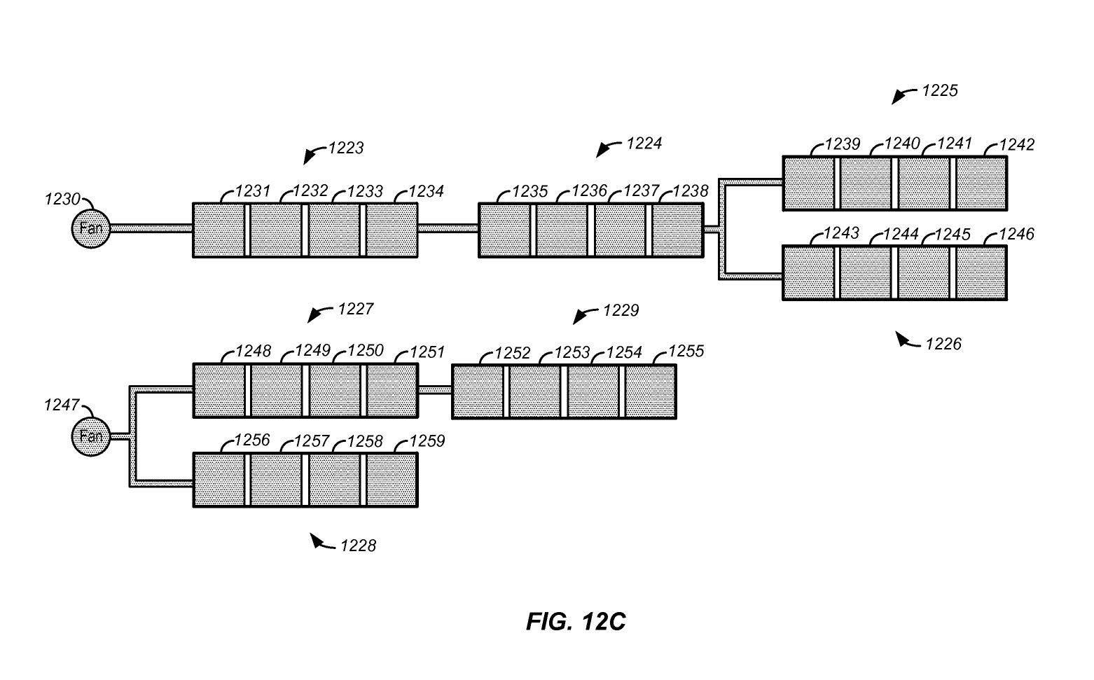

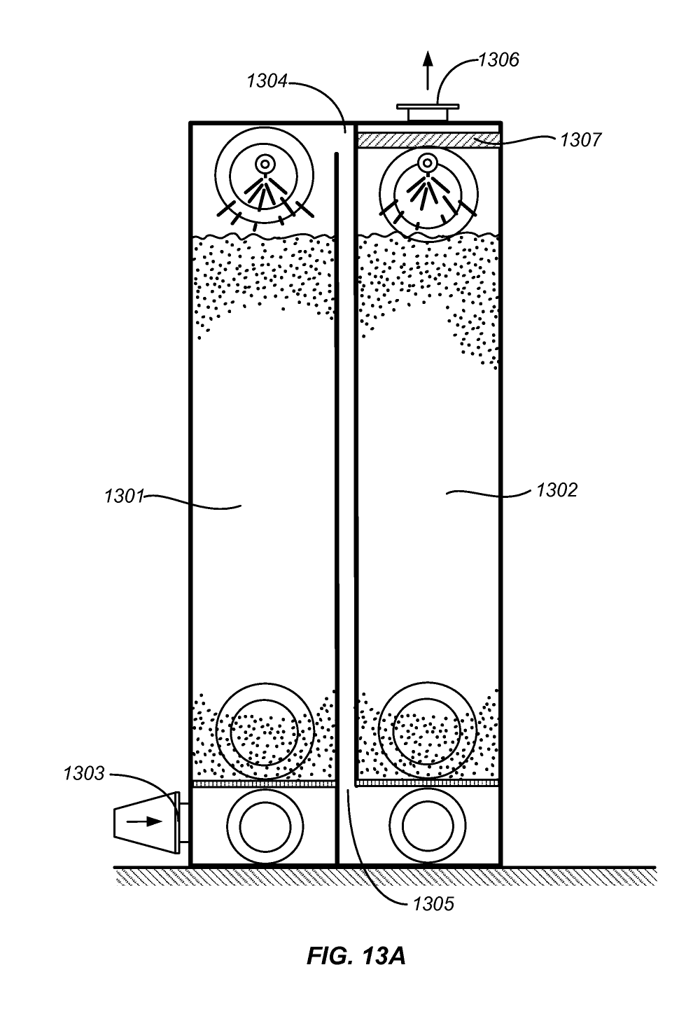

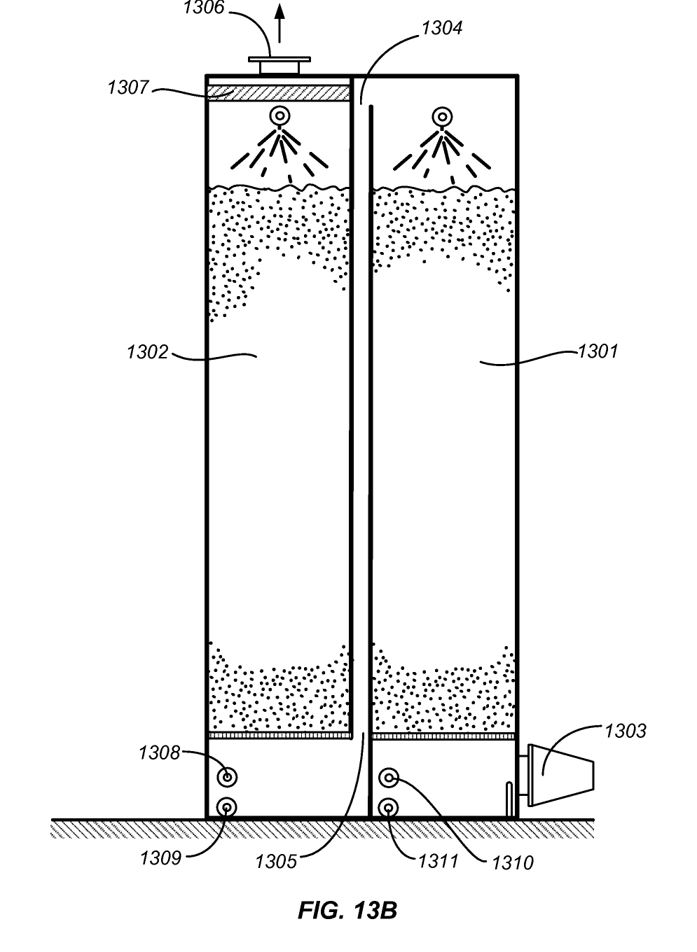

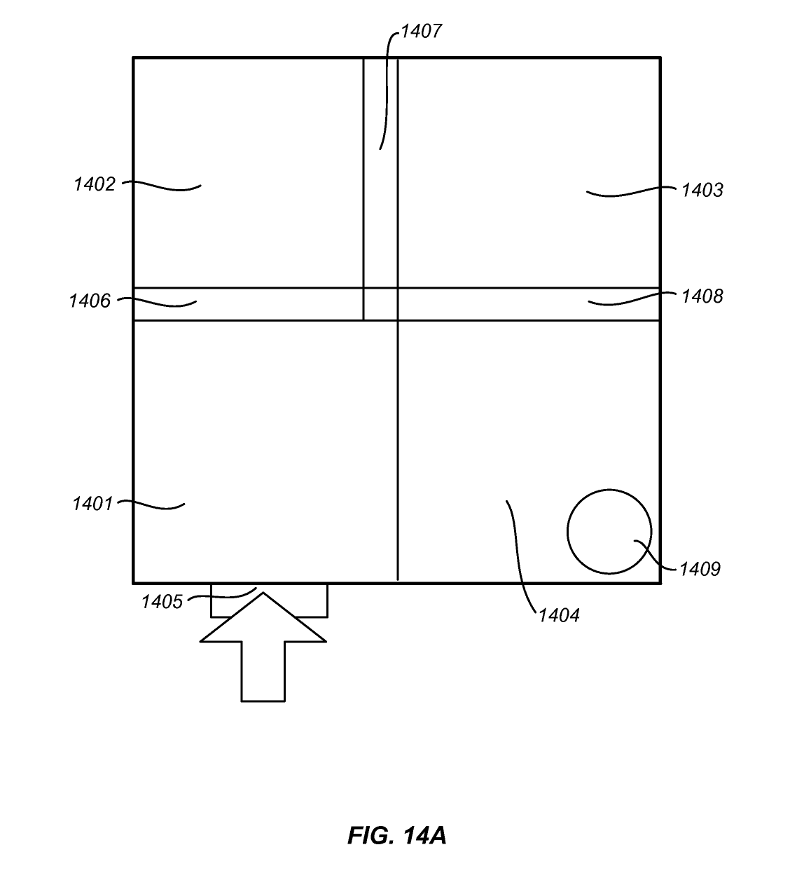

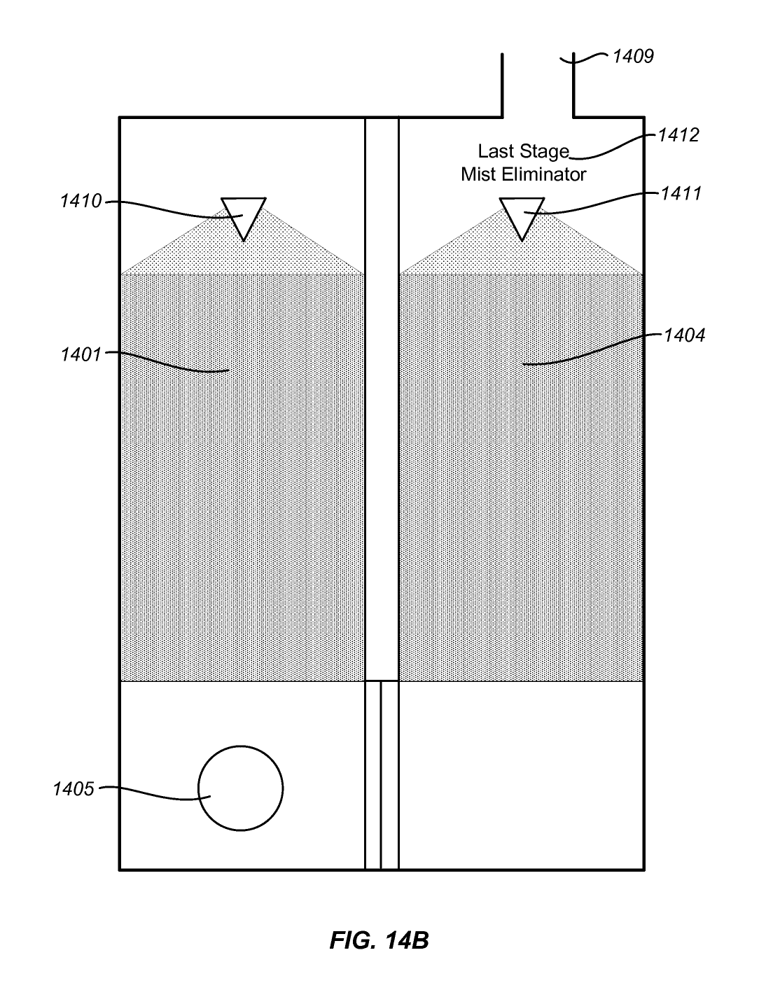

A non-limiting example of the methods, devices and systems described herein is depicted in FIG. 12A, FIG. 12B, and FIG. 12C. As depicted in a non-limiting example in FIG. 12A, two four-stage reactors are aligned in series, the first reactor 1201 comprising a first absorber 1206, a second absorber 1207, a third absorber 1208, and a fourth absorber 1209, and the second reactor 1202 comprising a fifth absorber 1210, a sixth absorber 1211, a seventh absorber 1212, and an eighth absorber 1213, provided that the fourth absorber is operatively connected to fifth absorber and/or the second reactor and provided that a fan 1205 is operatively connected to the first absorber and/or the first reactor. As depicted in a non-limiting example in FIG. 12B, two four-stage reactors are aligned in parallel, the first reactor 1203 comprising a first absorber 1215, a second absorber 1216, a third absorber 1217, and a fourth absorber 1218, and the second reactor 1204 comprising a fifth absorber 1219, a sixth absorber 1220, a seventh absorber 1221, and an eighth absorber 1222, provided that a fan 1214 is operatively connected to the first absorber and/or the first reactor and the fan is operatively connected to the fifth absorber and/or the second reactor. As depicted in a non-limiting example in FIG. 12C, four four-stage rectors aligned in a combination of in series an in parallel, the first reactor 1223 comprising a first absorber 1231, a second absorber 1232, a third absorber 1233, and a fourth absorber 1233, the second reactor 1224 aligned in series with the first reactor and comprising a fifth absorber 1235, a sixth absorber 1236, a seventh absorber 1237, and an eighth absorber 1238, the third reactor 1225 aligned in series with the first and second reactors and comprising a ninth absorber 1239, a tenth absorber 1240, an eleventh absorber 1241, and a twelfth absorber 1242, and a fourth reactor 1226 aligned in series with the first and second reactors and in parallel with the third reactor, the fourth reactor comprising a thirteenth absorber 1243, a fourteenth absorber 1244, a fifteenth absorber 1245, and a sixteenth absorber 1246, provided that a fan 1230 is operatively connected to the first reactor and/or the first absorber. As depicted in a non-limiting example in FIG. 12C, four four-stage rectors aligned in a combination of in series an in parallel, the first reactor 1227 comprising a first absorber 1248, a second absorber 1249, a third absorber 1250, and a fourth absorber 1251, the second reactor 1228 aligned in parallel with the first absorber and comprising a fifth absorber 1256, a sixth absorber 1257, a seventh absorber 1258, and an eighth absorber 1259, and the third reactor 1229 connected in series with the first reactor and comprising a ninth reactor 1252, a tenth reactor 1253, an eleventh reactor 1254, and a twelfth reactor 1255, provided that a fan 1247 is operatively connected to the first absorber and/or the first reactor and the fan is operatively connected to the fifth absorber and/or the second reactor. In some embodiments, two consecutive absorbers in a reactor are configured as described in FIGS. 13A and B. In some embodiments, four consecutive absorbers are configured as described in FIGS. 14A and B.

A non-limiting example of the methods, devices and systems described herein is depicted in FIG. 13A and FIG. 13B. In some embodiments, as depicted in FIG. 13A and FIG. 13B a first absorber 1301 and a second absorber 1302 are aligned in series. In some embodiments, FIG. 13A and FIG. 13B depicts a non-limiting example of a two-stage reactor. FIG. 13A depicts a front view of the absorbers and FIG. 13B depicts a rear view of the absorbers. In some embodiments the first absorber 1301 comprises a first exhaust inlet 1303 and a first exhaust outlet 1304 operatively connected the second exhaust inlet 1305 of the second absorber 1302. In some embodiments the second absorber comprises a mist eliminator 1307 and a second exhaust outlet 1306. In some embodiments, the first absorber comprises a first liquid waste stream outlet 1308 and a first service drain 1309. In some embodiments, the second absorber comprises a second liquid waste stream outlet 1310 and a second service drain 1311.

A non-limiting example of the methods, devices and systems described herein is depicted in FIG. 14A and FIG. 14B. In some embodiments, as depicted in FIG. 14A and FIG. 14B, a first absorber 1401, a second absorber 1402, a third absorber 1403, and a fourth absorber 1404 are aligned in series. In some embodiments, FIG. 14A depicts a non-limiting example of a four-stage reactor. FIG. 14A depicts a top view of the absorbers, and FIG. 14B depicts a front view of the absorbers. In some embodiments the first absorber 1401 comprises a first exhaust inlet 1405. In some embodiments the exhaust flows up through the first absorber, down a first exhaust duct 1406 so as to enter the second absorber 1402, up through the second absorber and down a second exhaust duct 1407 so as to enter the third absorber 1403, up through the third absorber and down a third exhaust duct 1408 so as to enter the fourth absorber 1404 and exits the exhaust outlet 1409 in the fourth absorber. In some embodiments, the first absorber 1401 comprises a component 1410 configured to spray or deliver liquid waste to the interior of the absorber. In some embodiments, the fourth absorber 1404 comprises a component 1411 configured to spray or deliver liquid waste to the interior of the absorber. In some embodiments, the fourth absorber comprises a mist eliminator 1412.

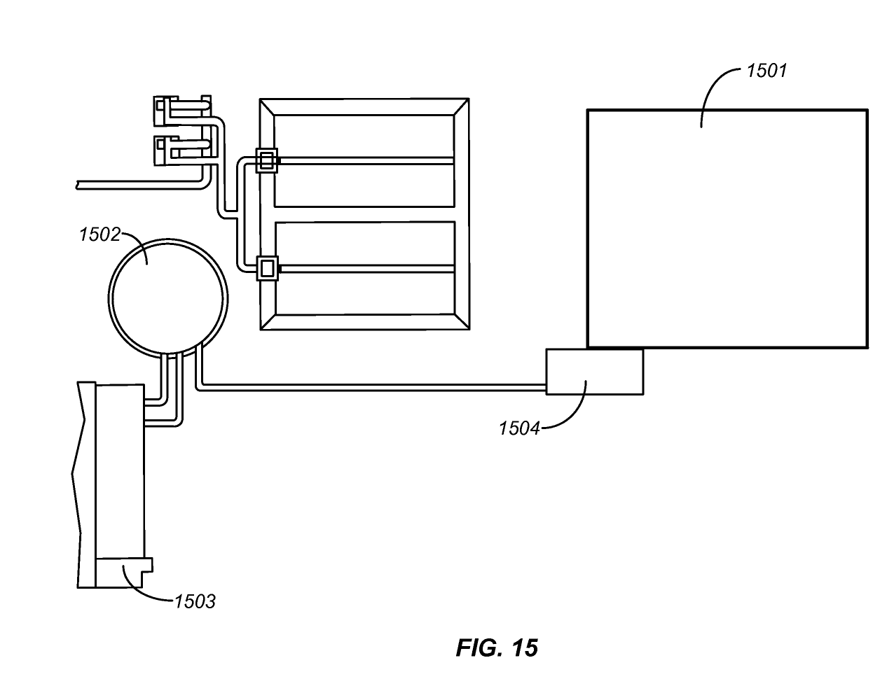

A non-limiting example of the methods, devices and systems described herein as depicted in the elevation view in FIG. 15 comprises a device room 1501, a tank 1502, a dewatering building 1503 and a system area 1504 comprising absorbers, reactors, a sump pump and an oxidant source. In some embodiments the tank is a centrate tank. In some embodiments, the system area further comprises a quencher and/or a cooler and/or a fan. In some embodiments the device room comprises one or more of a biogas engine, an internal combustion engine, a boiler, a turbine or and/or a microturbine. In some embodiments, the system area comprises a control room.

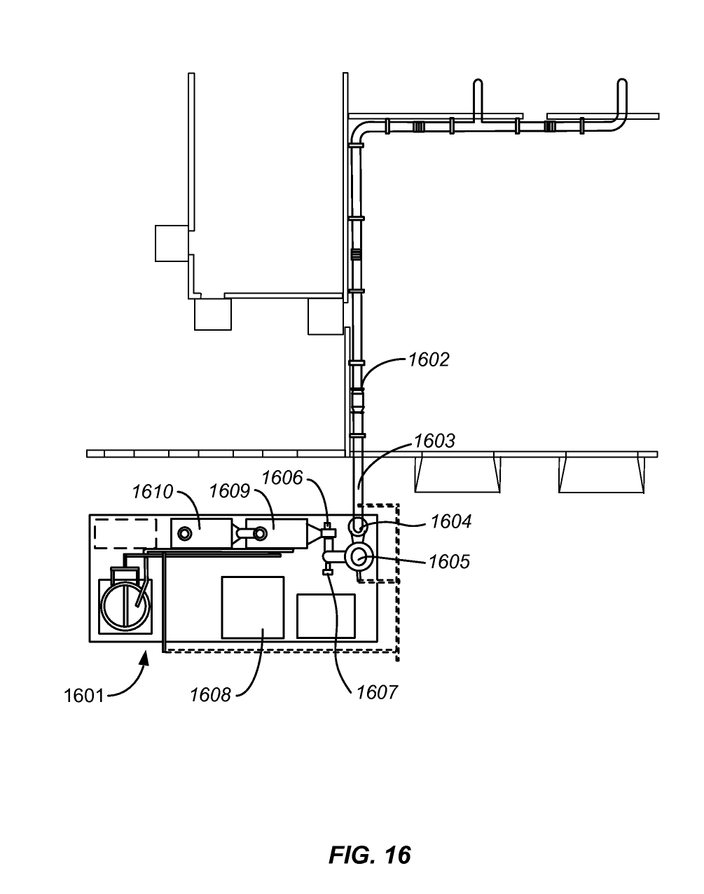

A non-limiting example of the methods, devices and systems described herein as depicted in the elevation view in FIG. 16 comprises a system area 1601 comprising a first absorber 1609, a second absorber 1610, a quencher 1604, a cooler 1605, a fan 1606, and an oxidant source 1608. In some embodiments 1609 depicts a first reactor. In some embodiments 1610 depicts a second reactor. In some embodiments, the quencher, the cooler, the fan, the first absorber, and the second absorber are operatively connected. In some embodiments, the quencher, the cooler, the fan, the first reactor, and the second reactor are operatively connected. In some embodiments the quencher is operatively connected 1603 to the exhaust outlet of the first and/or the second device so as to deliver exhaust to the quencher. In some embodiments an oxidant catalyst 1602 is operatively connected to the system area and/or the quencher. In some embodiments an oxidant inlet 1607 is located between the cooler and the first absorber. In some embodiments an oxidant inlet 1607 is located between the cooler and the first reactor. In some embodiments, the oxidant source 1608 generates ozone. In some embodiments, the oxidant source is operatively connected to the oxidant inlet 1607.

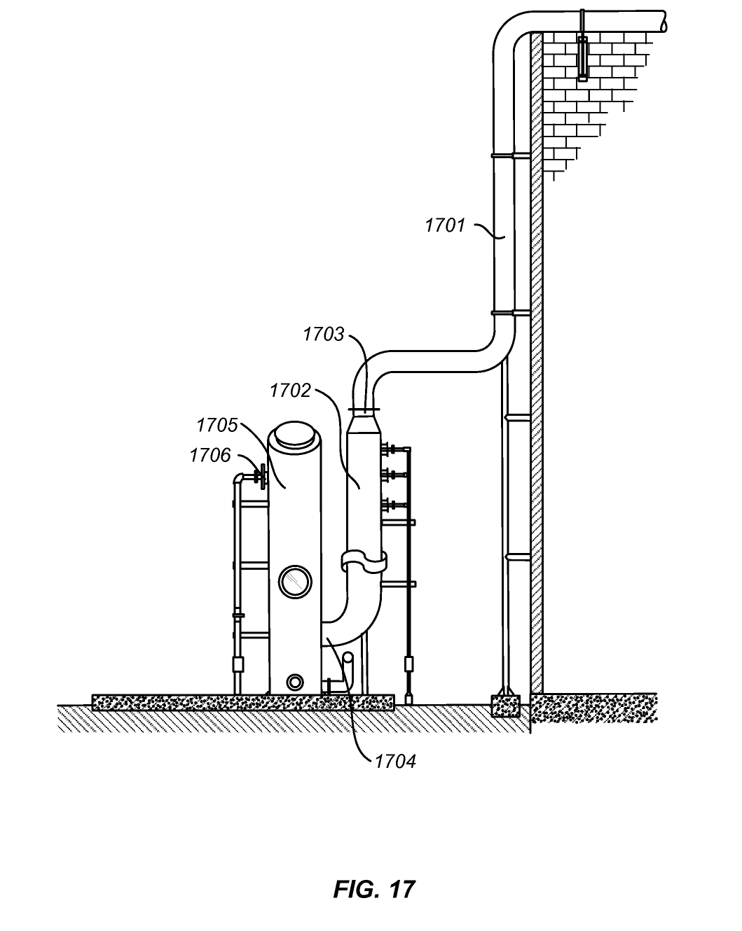

A non-limiting example of the methods, devices and systems described herein as depicted in FIG. 17 comprises a quencher 1702 and a cooler 1705, the quencher and cooler being operatively connected. In some embodiments the quencher is operatively connected 1701 to the exhaust outlet of a device so as to deliver exhaust to the quencher. In some embodiments, the quencher comprises a quencher exhaust inlet 1703 and a quencher exhaust outlet 1704 operatively connected to the cooler 1705. In some embodiments, the cooler comprises a liquid water inlet 1706.

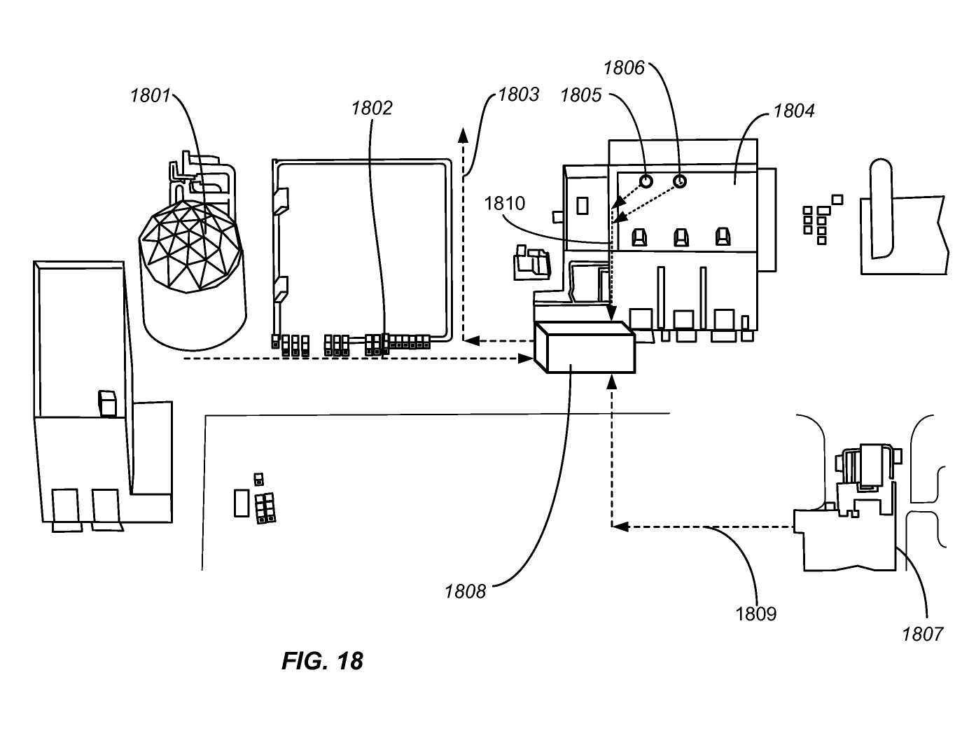

A non-limiting example showing implementation of the platforms, methods, devices and systems in a wastewater treatment facility is depicted in FIG. 18. In some embodiments, the implementation of the platforms, methods, devices and systems comprises a tank 1801 configured to hold liquid waste. In some embodiments the tank 1801 is a centrate tank. In some embodiments, the tank is operatively connected 1802 to the area 1808. In some embodiments, the implementation of the platforms, methods, devices and systems comprises a device area 1804 configured with exhaust outlets 1805 and 1806. In some embodiments, the exhaust outlets 1805 and 1806 are operatively connected 1810 to the area 1808. In some embodiments, the implementation of the platforms, methods, devices and systems comprises an area 1808 comprising one or more sumps, sump pumps, absorbers, reactors, quenchers, coolers, fans, and/or oxidant sources. In some embodiments the area 1808 is operatively connected to a drain line 1803. In some embodiments, the implementation of the platforms, methods, devices and systems comprises a chlorine mixing station 1807. In some embodiments, the chlorine mixing station 1807 is operatively connected 1809 to the area 1808. In some embodiments the device area 1804 comprises one or more of a biogas engine, an internal combustion engine, a boiler, a turbine or and/or a microturbine. In some embodiments, the area 1808 comprises a control room.

### Systems, Methods, Platforms, and Devices

One feature of the subject matter described herein is a method of decreasing NOx emissions in an engine exhaust comprising contacting the engine exhaust from an engine, such as a combustion engine, with the liquid waste stream from the biogas production unit to generate a treated exhaust with decreased NOx emissions. In some embodiments, the engine exhaust is contacted with the liquid waste stream in a single absorber. In further or additional embodiments, the engine exhaust is contacted with the liquid waste in at least two absorbers operatively connected in parallel and/or successively (e.g., in-line/series). In further or additional embodiments, the engine exhaust is contacted with the liquid waste in at least three absorbers operatively connected in parallel and/or successively (e.g., in-line/series). In still further or additional embodiments, the engine exhaust is contacted with the liquid waste in at least four or more absorbers operatively connected in parallel and/or successively (e.g., in-line/series).

In one aspect of the systems, methods and devices described herein is a system comprising: a reactor comprising a first absorber comprising a first liquid waste stream inlet a first exhaust inlet, and a first exhaust outlet; a biogas production unit constructed so as to convert waste to a biogas stream and a liquid waste stream and comprising a biogas stream outlet and a liquid waste stream outlet operatively connected to the first liquid waste stream inlet of the first absorber; and a device constructed to utilize the biogas stream to produce energy and an exhaust comprising a gas, the device comprising a biogas stream inlet operatively connected to the biogas stream outlet of the biogas production unit and an exhaust outlet operatively connected to the first exhaust inlet of the first absorber, provided that the reactor is constructed so as to treat exhaust so as to decrease or reduce emissions or pollutants by contacting the exhaust with the liquid waste stream from the biogas production unit. In some embodiments the reactor further comprises a second absorber comprising a second liquid waste stream inlet, a second exhaust inlet, and a second exhaust outlet, provided that the liquid waste stream outlet of the biogas production unit is operatively connected to the first liquid waste stream inlet of the first absorber and the second liquid waste stream inlet of the second absorber, and the first exhaust outlet of the first absorber is operatively connected to the second exhaust inlet of the second absorber. In some embodiments the exhaust comprises a gas comprising one or more of NOx, SOx, or COx. In some embodiments the gas comprises one or more of NOx, SOx, or COx. In some embodiments the reactor further comprises a plurality of absorbers, provided that all absorbers are operatively connected. In some embodiments the reactor further comprises a plurality of absorbers aligned in parallel, provided that each absorber comprises a liquid waste stream inlet, an exhaust inlet and an exhaust outlet, and provided that the liquid waste stream outlet of the biogas production unit is operatively connected to the liquid waste stream inlet of each absorber and the exhaust outlet of the device is operatively connected to the exhaust inlet of each absorber. In some embodiments the reactor is entirely modular (i.e. one or more absorbers are interchangeable and/or additional absorbers are added to or removed from the reactor). In some embodiments the reactor comprises more than one absorber aligned in series, aligned in parallel, and/or aligned in a combination of in series and in parallel. In some embodiments the reactor is rectangular or square. In some embodiments a liquid is anything that flows. In some embodiments, the device comprises a combustion engine, a boiler, a turbine or and/or a microturbine. In some embodiments the liquid waste is stored in a tank prior to entering one or more absorbers. In some embodiments, the liquid waste is delivered from the tank to a sump and a sump pump is used to deliver the liquid waste to one or more absorbers. In some embodiments, the liquid waste gravity flows through the one or more absorbers and back to the sump. In some embodiments the tank is a centrate tank.

In one aspect of the systems, methods and devices described herein is a reactor comprising a first absorber comprising a first liquid waste stream inlet a first exhaust inlet, and a first exhaust outlet; a biogas production unit constructed so as to convert waste to a biogas stream and a liquid waste stream and comprising a biogas stream outlet and a liquid waste stream outlet operatively connected to the first liquid waste stream inlet of the first absorber; and a device constructed to utilize the biogas stream to produce energy and an exhaust comprising a gas, the device comprising a biogas stream inlet operatively connected to the biogas stream outlet of the biogas production unit and an exhaust outlet operatively connected to the first exhaust inlet of the first absorber, provided that the reactor is constructed so as to treat exhaust so as to decrease or reduce emissions or pollutants by contacting the exhaust with the liquid waste stream from the biogas production unit. In some embodiments the reactor further comprises a second absorber comprising a second liquid waste stream inlet, a second exhaust inlet, and a second exhaust outlet, provided that the liquid waste stream outlet of the biogas production unit is operatively connected to the first liquid waste stream inlet of the first absorber and the second liquid waste stream inlet of the second absorber, and the first exhaust outlet of the first absorber is operatively connected to the second exhaust inlet of the second absorber. In some embodiments the exhaust comprises a gas comprising one or more of NOx, SOx, or COx. In some embodiments the gas comprises one or more of NOx, SOx, or COx. In some embodiments the reactor further comprises a plurality of absorbers, provided that all absorbers are operatively connected. In some embodiments the reactor further comprises a plurality of absorbers aligned in parallel, provided that each absorber comprises a liquid waste stream inlet, an exhaust inlet and an exhaust outlet, and provided that the liquid waste stream outlet of the biogas production unit is operatively connected to the liquid waste stream inlet of each absorber and the exhaust outlet of the device is operatively connected to the exhaust inlet of each absorber. In some embodiments the reactor is entirely modular (i.e. one or more absorbers are interchangeable and/or additional absorbers are added to or removed from the reactor). In some embodiments the reactor comprises more than one absorber aligned in series, aligned in parallel, and/or aligned in a combination of in series and in parallel. In some embodiments the reactor is rectangular or square. In some embodiments a liquid is anything that flows. In some embodiments, the device comprises a combustion engine, a boiler, a turbine or and/or a microturbine. In some embodiments the liquid waste is stored in a tank prior to entering one or more absorbers. In some embodiments, the liquid waste is delivered from the tank to a sump and a sump pump is used to deliver the liquid waste to one or more absorbers. In some embodiments, the liquid waste gravity flows through the one or more absorbers and back to the sump.

In one aspect of the systems, methods and devices described herein is a method to treat exhaust comprising: providing a system comprising: a reactor comprising a first absorber comprising a first liquid waste stream inlet a first exhaust inlet, and a first exhaust outlet; a biogas production unit constructed so as to convert waste to a biogas stream and a liquid waste stream and comprising a biogas stream outlet and a liquid waste stream outlet operatively connected to the first liquid waste stream inlet of the first absorber; and a device constructed to utilize the biogas stream to produce energy and an exhaust comprising a gas, the device comprising a biogas stream inlet operatively connected to the biogas stream outlet of the biogas production unit and an exhaust outlet operatively connected to the first exhaust inlet of the first absorber, provided that the reactor is constructed so as to treat exhaust so as to decrease or reduce emissions or pollutants by contacting the exhaust with the liquid waste stream from the biogas production unit. In some embodiments the reactor further comprises a second absorber comprising a second liquid waste stream inlet, a second exhaust inlet, and a second exhaust outlet, provided that the liquid waste stream outlet of the biogas production unit is operatively connected to the first liquid waste stream inlet of the first absorber and the second liquid waste stream inlet of the second absorber, and the first exhaust outlet of the first absorber is operatively connected to the second exhaust inlet of the second absorber. In some embodiments the exhaust comprises a gas comprising one or more of NOx, SOx, or COx. In some embodiments the gas comprises one or more of NOx, SOx, or COx. In some embodiments the reactor further comprises a plurality of absorbers, provided that all absorbers are operatively connected. In some embodiments the reactor further comprises a plurality of absorbers aligned in parallel, provided that each absorber comprises a liquid waste stream inlet, an exhaust inlet and an exhaust outlet, and provided that the liquid waste stream outlet of the biogas production unit is operatively connected to the liquid waste stream inlet of each absorber and the exhaust outlet of the device is operatively connected to the exhaust inlet of each absorber. In some embodiments the reactor is entirely modular (i.e. one or more absorbers are interchangeable and/or additional absorbers are added to or removed from the reactor). In some embodiments the reactor comprises more than one absorber aligned in series, aligned in parallel, and/or aligned in a combination of in series and in parallel. In some embodiments the reactor is rectangular or square. In some embodiments a liquid is anything that flows. In some embodiments, the device comprises a combustion engine, a boiler, a turbine or and/or a microturbine. In some embodiments the liquid waste is stored in a tank prior to entering one or more absorbers. In some embodiments, the liquid waste is delivered from the tank to a sump and a sump pump is used to deliver the liquid waste to one or more absorbers. In some embodiments, the liquid waste gravity flows through the one or more absorbers and back to the sump.

Another feature of the subject matter provided herein is a biogas production unit that converts waste into a biogas stream and a liquid waste. An engine, such as a combustion engine, turbine, or boiler, utilizes the biogas stream from the biogas production unit to produce energy and an engine exhaust. The biogas production unit provides liquid waste to the absorber. The absorber is operatively connected to a liquid waste source and an engine exhaust source. The system defined above is a wet scrubbing system in certain applications. The engine exhaust, in certain applications, contains nitrogenous compounds such as NOx. As used herein, “NOx” means oxides of nitrogen including but not limited to NO, NO₂, N₂O₃, N₂O₄ and N₂O₅. As used herein, “SOx” means oxides of sulfur. As used herein, “COx” means oxides of carbon.

In some embodiments, the biogas production unit is an anaerobic digester or landfill. In further or additional embodiments, the engine is a combustion engine. The combustion engine, in certain applications, is a biogas engine, internal combustion engine, turbine, microturbine, or boiler.

In some embodiments, components of the systems, methods, devices described herein, operatively connected comprises a direct connection or indirect connection. In some embodiments, the waste is subjected to a screening and grit removal process prior to entering the biogas production unit. In some embodiments, the biogas production unit comprises anaerobic bacteria. In some embodiments, the anaerobic bacteria convert the waste to an inert material, for example digested sludge, and biogas, for example methane gas and carbon dioxide. In some embodiments, before entering the device, the biogas is conditioned so as to remove moisture, sulfur compounds and siloxanes. In some embodiments, the digested sludge from the digesters is transferred to one or more centrifuges. In some embodiments, the one or more centrifuges are configured to separate the digested sludge solids (cake) from the liquid (centrate). In some embodiments separation of digested sludge solids (cake) from the liquid (centrate) is accomplished with screens and/or other types of filters. In some embodiments, the liquid (centrate) is stored in a tank after being separated from the sludge solids (cake). In some embodiments, a sump is located between the tank and one or more absorbers. In some embodiments, the liquid (centrate) is delivered to the sump and a sump pump is used to deliver the liquid (centrate) to the one or more absorbers. In some embodiments, the liquid (centrate) is delivered to the top of one or more absorbers. In some embodiments, the liquid (centrate) gravity flows through the one or more absorbers and back to the sump. In some embodiments, liquid (centrate) continually flows from the tank to the sump. In some embodiments liquid (centrate) continually flows from the sump and to a headwork of a wastewater treatment facility.

### Quencher

In some embodiments, a quencher is provided, for example in a manner that is operatively connected to an engine, for example a combustion engine. The quencher is not necessarily included for proper function of the systems, methods and devices described herein.

In some embodiments of the systems, methods and devices described herein, a quencher is provided such that the quencher is operatively connected to an energy producing device that generates exhaust, the energy producing device comprising a biogas engine, an internal combustion engine, a turbine, a microturbine or a gas fired boiler, and provided that the quencher accesses and cools the exhaust to a temperature that is less than or equal to about 90° F., about 100° F., about 110° F., about 120° F., about 130° F., about 140° F., or about 150° F.

In some embodiments of the systems, methods and devices described herein, hot biogas engine exhaust is saturated with water vapor and cooled to a temperature that is less than or equal to about 90° F., about 100° F., about 110° F., about 120° F., about 130° F., about 140° F., or about 150° F. prior to the oxidation and wet scrubbing steps. The exhaust exits the engine at approximately about 500° F., about 600° F., about 650° F., about 700° F., about 750° F., or about 800° F., about 850° F., or about 900° F. In some embodiments, the engine exhaust is saturated with either plant water or water from the primary/secondary clarifier within the quencher. Some initial cooling of the exhaust also takes place within the quencher.

In some embodiments, a quencher and a cooler system simultaneously cools and saturates the exhaust with water. After the quencher and cooler system, the exhaust is cooled to approximately 100-120° F. (or lower) and suitably conditioned for rapid and selective NO oxidation using ozone. In some embodiments, a mixer is operatively connected to the quencher, cooler, or both the quencher and the cooler. In some embodiments a plurality of mixers are operatively connected to the quencher, cooler, or both the quencher and the cooler

### Cooler

In some embodiments, a cooler is provided, and in certain further or additional embodiments, the cooler is operatively connected to a quencher. In some embodiments, a mixer is operatively connected to the quencher, cooler, or both the quencher and the cooler. In some embodiments a plurality of mixers are operatively connected to the quencher, cooler, or both the quencher and the cooler. In some embodiments, provided are multiple absorbers arranged successively or in parallel. For example, in some embodiments, the absorbers are arranged in parallel groups of multiple absorbers. The cooler is not necessarily included for proper function of the systems, methods and devices described herein.

For example, in some embodiments, hot biogas engine exhaust is saturated with water vapor and cooled to a temperature that is less than or equal to about 90° F., about 100° F., about 110° F., about 120° F., about 130° F., about 140° F., or about 150° F. prior to the oxidation and wet scrubbing steps. In some embodiments, the saturated engine exhaust undergoes cooling to a temperature that is less than or equal to about 90° F., about 100° F., about 110° F., about 120° F., about 130° F., about 140° F., or about 150° F. within the Cooler. In some embodiments, this is accomplished through the use of either digester effluent and/or a primary or secondary clarifier water within the cooler. As discussed herein, the instant inventors have discovered that all SO₂ is effectively removed by the cooler using this method, as illustrated by table below:

TABLE 1
SO₂ Levels before and after Cooling

| SO₂ before | SO₂ after | | |
|---|---|---|---|
| cooler | cooler | Engine output | Cooler dimensions |
| 726 ppm | 0 ppm | 150 kW/600 acfm | 8′ × 2′ Lanpac 2.3″ |

In some embodiments, a quencher and cooler system is simultaneously used to cool and saturate exhaust with an aqueous solution, such as water. After the quencher and cooler system, exhaust is cooled to approximately about 60° F., or about 70° F., or about 80° F., or about 90° F., or about 100° F., or about 110° F., or about 120° F., or about 130° F., or about 140° F., or about 150° F. and suitably conditioned for rapid and selective NO oxidation using ozone. In some embodiments, a mixer is operatively connected to the quencher, cooler, or both the quencher and the cooler. In some embodiments a plurality of mixers are operatively connected to the quencher, cooler, or both the quencher and the cooler.

### Multi-Stage Wet Scrubber (Absorber Array/Plurality)

Embodiments of the system described herein incorporate one absorber or multiple absorbers. In some embodiments of the systems, methods and devices described herein, one absorber, two absorbers, three absorbers, four absorbers or a plurality of absorbers are included. The wet scrubbing system described herein, in certain embodiments, comprises a first absorber that is operatively connected to (a) a biogas production unit that converts waste to a biogas stream and a liquid waste stream, for example further comprising a biogas stream outlet and a liquid waste stream outlet that connects to a liquid waste stream inlet of the first absorber; and (b) an engine, for example, a combustion engine that utilizes the biogas stream to produce energy and an engine exhaust stream containing NOx and optionally further comprising a biogas stream inlet and an engine exhaust stream outlet connected to the engine exhaust inlet of the first absorber; provided that a second absorber is displaced within the system in-line with or in parallel with the first absorber.

In one aspect of the systems, methods and devices described herein is a system comprising: a reactor comprising a first absorber comprising a first liquid waste stream inlet, a first exhaust inlet and a first exhaust outlet; a biogas production unit constructed so as to convert waste to a biogas stream and a liquid waste stream and comprising a biogas stream outlet and a liquid waste stream outlet operatively connected to the first liquid waste stream inlet of the first absorber; and a device constructed to utilize the biogas stream to produce energy and an exhaust comprising a gas, the device comprising a biogas stream inlet operatively connected to the biogas stream outlet of the biogas production unit and an exhaust outlet operatively connected to the first exhaust inlet of the first absorber; provided that the reactor is constructed so as to treat exhaust so as to decrease or reduce emissions or pollutants by contacting the exhaust with the liquid waste stream from the biogas production unit. In some embodiments the reactor further comprises a second absorber comprising a second liquid waste stream inlet, a second exhaust inlet and a second exhaust outlet, provided that the liquid waste stream outlet of the biogas production unit is operatively connected to the first liquid waste stream inlet of the first absorber and the second liquid waste stream inlet of the second absorber, and provided that the exhaust outlet of the biogas production unit is operatively connected to the first exhaust inlet of the first absorber and the first exhaust outlet of the first absorber is operatively connected to the second exhaust inlet of the second absorber. In some embodiments the reactor further comprises a second absorber comprising a second liquid waste stream inlet, a second exhaust inlet and a second exhaust outlet and a third absorber comprising a third liquid waste stream inlet, a third exhaust inlet and a third exhaust outlet, provided that the liquid waste stream outlet of the biogas production unit is operatively connected to the first liquid waste stream inlet of the first absorber, the second liquid waste stream inlet of the second absorber, and the third liquid waste stream inlet of the third absorber, and provided that the exhaust outlet of the biogas production unit is operatively connected to the first exhaust inlet of the first absorber, the first exhaust outlet of the first absorber is operatively connected to the second exhaust inlet of the second absorber, and the second exhaust outlet of the second absorber is operatively connected to third the exhaust inlet of the third absorber. In some embodiments the reactor further comprises a second absorber comprising a second liquid waste stream inlet, a second exhaust inlet and a second exhaust outlet, a third absorber comprising a third liquid waste stream inlet, a third exhaust inlet and a third exhaust outlet and a fourth absorber comprising a fourth liquid waste stream inlet, a fourth exhaust inlet and a fourth exhaust outlet, provided that the liquid waste stream outlet of the biogas production unit is operatively connected to the first liquid waste stream inlet of the first absorber, the second liquid waste stream inlet of the second absorber, the third liquid waste stream inlet of the third absorber, and the fourth liquid waste stream inlet of the fourth absorber, and provided that the exhaust outlet of the biogas production unit is operatively connected to the first exhaust inlet of the first absorber, the first exhaust outlet of the first absorber is operatively connected to the second exhaust inlet of the second absorber, the second exhaust outlet of the second absorber is operatively connected to third the exhaust inlet of the third absorber, and the third exhaust outlet of the third absorber is operatively connected to fourth the exhaust inlet of the fourth absorber. In some embodiments the reactor further comprises a plurality of absorbers, provided that the exhaust outlet of one absorber is operatively connected to the exhaust inlet of another absorber. In some embodiments the exhaust comprises a gas comprising one or more of NOx, SOx, or COx. In some embodiments the gas comprises one or more of NOx, SOx, or COx. In some embodiments the reactor further comprises a plurality of absorbers, provided that all absorbers are operatively connected. In some embodiments the reactor further comprises a plurality of absorbers aligned in parallel, provided that each absorber comprises a liquid waste stream inlet, an exhaust inlet and an exhaust outlet, and provided that the liquid waste stream outlet of the biogas production unit is operatively connected to the liquid waste stream inlet of each absorber and the exhaust outlet of the device is operatively connected to the exhaust inlet of each absorber. In some embodiments the system is entirely modular (i.e. one or more reactors, absorbers, biogas production units, devices constructed to utilize the biogas stream to produce energy and an exhaust comprising a gas, fans, mist eliminators and/or other components are added or removed from the system). In some embodiments the reactor is entirely modular (i.e. one or more absorbers are interchangeable and/or additional absorbers are added to or removed from the reactor). In some embodiments the reactor comprises more than one absorber aligned in series, aligned in parallel, and/or aligned in a combination of in series and in parallel. In some embodiments the reactor comprises a first absorber and a second absorber aligned in parallel, provided that the first absorber and the second absorber are each operatively connected to the exhaust outlet of the biogas production unit. In some embodiments the reactor is rectangular or square. In some embodiments a liquid is anything that flows. In some embodiments, the device comprises a combustion engine, a boiler, a turbine or and/or a microturbine.

In one aspect of the systems, methods and devices described herein is a system comprising: a reactor comprising a first absorber comprising a first liquid waste stream inlet, a first exhaust inlet and a first exhaust outlet; a biogas production unit constructed so as to convert waste to a biogas stream and a liquid waste stream and comprising a biogas stream outlet and a liquid waste stream outlet operatively connected to the first liquid waste stream inlet of the first absorber; and a device constructed to utilize the biogas stream to produce energy and an exhaust comprising a gas, the device comprising a biogas stream inlet operatively connected to the biogas stream outlet of the biogas production unit and an exhaust outlet operatively connected to the first exhaust inlet of the first absorber, provided that the reactor is constructed so as to treat exhaust so as to decrease or reduce emissions or pollutants by contacting the exhaust with the liquid waste stream from the biogas production unit. In some embodiments the exhaust comprises a gas comprising one or more of NOx, SOx, or COx. In some embodiments the gas comprises one or more of NOx, SOx, or COx. In some embodiments the reactor comprises more than one absorber aligned in series, aligned in parallel, and/or aligned in a combination of in series and in parallel. In some embodiments the reactor comprises a first absorber and a second absorber aligned in parallel, the first absorber comprising a first liquid waste stream inlet, a first exhaust inlet and a first exhaust outlet and the second absorber comprising a second liquid waste stream inlet, a second exhaust inlet and a second exhaust outlet, provided that the liquid waste stream outlet of the biogas production unit is operatively connected to the first liquid waste stream inlet of the first absorber and the second liquid waste stream inlet of the second absorber, the first exhaust inlet of the first absorber is operatively connected to the exhaust outlet of the biogas production unit, and the second exhaust inlet of the second absorber is operatively connected to the exhaust outlet of the biogas production unit. In some embodiments the system comprises a fan and two reactors aligned in series, the first reactor comprising: a first absorber comprising a first liquid waste stream inlet, a first exhaust inlet and a first exhaust outlet; a second absorber comprising a second liquid waste stream inlet, a second exhaust inlet and a second exhaust outlet; a third absorber comprising a third liquid waste stream inlet, a third exhaust inlet and a third exhaust outlet; and a fourth absorber comprising a fourth liquid waste stream inlet, a fourth exhaust inlet and a fourth exhaust outlet, and the second reactor comprising: a fifth absorber comprising a fifth liquid waste stream inlet, a fifth exhaust inlet and a fifth exhaust outlet; a sixth absorber comprising a sixth liquid waste stream inlet, a sixth exhaust inlet and a sixth exhaust outlet; a seventh absorber comprising a seventh liquid waste stream inlet, a seventh exhaust inlet and a seventh exhaust outlet; and an eighth absorber comprising a eighth liquid waste stream inlet, an eighth exhaust inlet and an eighth exhaust outlet, provided that the liquid waste stream outlet of the biogas production unit is operatively connected to the first liquid waste stream inlet of the first absorber, the second liquid waste stream inlet of the second absorber, the third liquid waste stream inlet of the third absorber, the fourth liquid waste stream inlet of the fourth absorber, the fifth liquid waste stream inlet of the fifth absorber, the sixth liquid waste stream inlet of the sixth absorber, the seventh liquid waste stream inlet of the seventh absorber, and the eighth liquid waste stream inlet of the eighth absorber, and provided that the first exhaust inlet of the first absorber is operatively connected to the fan, the first exhaust outlet of the first absorber is operatively connected to the second exhaust inlet of the second absorber, the second exhaust outlet of the second absorber is operatively connected to the third exhaust inlet of the third absorber, the third exhaust outlet of the third absorber is operatively connected to the fourth exhaust inlet of the fourth absorber, the fifth exhaust inlet of the fifth absorber is operatively connected to the first reactor and/or the fourth exhaust outlet of the fourth absorber, the fifth exhaust outlet of the fifth absorber is operatively connected to the sixth exhaust inlet of the sixth absorber, the sixth exhaust outlet of the sixth absorber is operatively connected to the seventh exhaust inlet of the seventh absorber, and the seventh exhaust outlet of the seventh absorber is operatively connected to the eighth exhaust inlet of the eighth absorber. In some embodiments the reactor is rectangular or square. In some embodiments a liquid is anything that flows. In some embodiments, the device comprises a combustion engine, a boiler, a turbine or and/or a microturbine.

In one aspect of the systems, methods and devices described herein is a system comprising: a reactor comprising a first absorber comprising a first liquid waste stream inlet, a first exhaust inlet and a first exhaust outlet; a biogas production unit constructed so as to convert waste to a biogas stream and a liquid waste stream and comprising a biogas stream outlet and a liquid waste stream outlet operatively connected to the first liquid waste stream inlet of the first absorber; and a device constructed to utilize the biogas stream to produce energy and an exhaust comprising a gas, the device comprising a biogas stream inlet operatively connected to the biogas stream outlet of the biogas production unit and an exhaust outlet operatively connected to the first exhaust inlet of the first absorber; provided that the reactor is constructed so as to treat exhaust so as to decrease or reduce emissions or pollutants by contacting the exhaust with the liquid waste stream from the biogas production unit. In some embodiments the exhaust comprises a gas comprising one or more of NOx, SOx, or COx. In some embodiments the gas comprises one or more of NOx, SOx, or COx. In some embodiments the reactor comprises more than one absorber aligned in series, aligned in parallel, and/or aligned in a combination of in series and in parallel. In some embodiments the system comprises a fan and two reactors aligned in parallel, the first reactor comprising: a first absorber comprising a first liquid waste stream inlet, a first exhaust inlet and a first exhaust outlet; a second absorber comprising a second liquid waste stream inlet, a second exhaust inlet and a second exhaust outlet; a third absorber comprising a third liquid waste stream inlet, a third exhaust inlet and a third exhaust outlet; and a fourth absorber comprising a fourth liquid waste stream inlet, a fourth exhaust inlet and a fourth exhaust outlet, and the second reactor comprising: a fifth absorber comprising a fifth liquid waste stream inlet, a fifth exhaust inlet and a fifth exhaust outlet; a sixth absorber comprising a sixth liquid waste stream inlet, a sixth exhaust inlet and a sixth exhaust outlet; a seventh absorber comprising a seventh liquid waste stream inlet, a seventh exhaust inlet and a seventh exhaust outlet; and an eighth absorber comprising an eighth liquid waste stream inlet, an eighth exhaust inlet and an eighth exhaust outlet, provided that the liquid waste stream outlet of the biogas production unit is operatively connected to the first liquid waste stream inlet of the first absorber, the second liquid waste stream inlet of the second absorber, the third liquid waste stream inlet of the third absorber, the fourth liquid waste stream inlet of the fourth absorber, the fifth liquid waste stream inlet of the fifth absorber, the sixth liquid waste stream inlet of the sixth absorber, the seventh liquid waste stream inlet of the seventh absorber, and the eighth liquid waste stream inlet of the eighth absorber, and provided that the first exhaust inlet of the first absorber is operatively connected to the fan, the first exhaust outlet of the first absorber is operatively connected to the second exhaust inlet of the second absorber, the second exhaust outlet of the second absorber is operatively connected to the third exhaust inlet of the third absorber, the third exhaust outlet of the third absorber is operatively connected to the fourth exhaust inlet of the fourth absorber, and provided that the fifth exhaust inlet of the fifth absorber is operatively connected to the fan, the fifth exhaust outlet of the fifth absorber is operatively connected to the sixth exhaust inlet of the sixth absorber, the sixth exhaust outlet of the sixth absorber is operatively connected to the seventh exhaust inlet of the seventh absorber, and the seventh exhaust outlet of the seventh absorber is operatively connected to the eighth exhaust inlet of the eighth absorber. In some embodiments the reactor is rectangular or square. In some embodiments a liquid is anything that flows. In some embodiments, the device comprises a combustion engine, a boiler, a turbine or and/or a microturbine.

In one aspect of the systems, methods and devices described herein is a reactor comprising a first absorber comprising: a first liquid waste stream inlet, a first exhaust inlet and a first exhaust outlet, the reactor operatively connected to: a biogas production unit constructed so as to convert waste to a biogas stream and a liquid waste stream and comprising a biogas stream outlet and a liquid waste stream outlet operatively connected to the first liquid waste stream inlet of the first absorber; and a device constructed to utilize the biogas stream to produce energy and an exhaust comprising a gas, the device comprising a biogas stream inlet operatively connected to the biogas stream outlet of the biogas production unit and an exhaust outlet operatively connected to the first exhaust inlet of the first absorber, provided that the reactor is constructed so as to treat exhaust so as to decrease or reduce emissions or pollutants by contacting the exhaust with the liquid waste stream from the biogas production unit. In some embodiments the reactor further comprises a second absorber comprising a second liquid waste stream inlet, a second exhaust inlet and a second exhaust outlet, provided that the liquid waste stream outlet of the biogas production unit is operatively connected to the first liquid waste stream inlet of the first absorber and the second liquid waste stream inlet of the second absorber, and provided that the exhaust outlet of the biogas production unit is operatively connected to the first exhaust inlet of the first absorber and the first exhaust outlet of the first absorber is operatively connected to the second exhaust inlet of the second absorber. In some embodiments the reactor further comprises a second absorber comprising a second liquid waste stream inlet, a second exhaust inlet and a second exhaust outlet and a third absorber comprising a third liquid waste stream inlet, a third exhaust inlet and a third exhaust outlet, provided that the liquid waste stream outlet of the biogas production unit is operatively connected to the first liquid waste stream inlet of the first absorber, the second liquid waste stream inlet of the second absorber, and the third liquid waste stream inlet of the third absorber, and provided that the exhaust outlet of the biogas production unit is operatively connected to the first exhaust inlet of the first absorber, the first exhaust outlet of the first absorber is operatively connected to the second exhaust inlet of the second absorber, and the second exhaust outlet of the second absorber is operatively connected to third the exhaust inlet of the third absorber. In some embodiments the reactor further comprises a second absorber comprising a second liquid waste stream inlet, a second exhaust inlet and a second exhaust outlet, a third absorber comprising a third liquid waste stream inlet, a third exhaust inlet and a third exhaust outlet and a fourth absorber comprising a fourth liquid waste stream inlet, a fourth exhaust inlet and a fourth exhaust outlet, provided that the liquid waste stream outlet of the biogas production unit is operatively connected to the first liquid waste stream inlet of the first absorber, the second liquid waste stream inlet of the second absorber, the third liquid waste stream inlet of the third absorber, and the fourth liquid waste stream inlet of the fourth absorber, and provided that the exhaust outlet of the biogas production unit is operatively connected to the first exhaust inlet of the first absorber, the first exhaust outlet of the first absorber is operatively connected to the second exhaust inlet of the second absorber, the second exhaust outlet of the second absorber is operatively connected to third the exhaust inlet of the third absorber, and the third exhaust outlet of the third absorber is operatively connected to fourth the exhaust inlet of the fourth absorber. In some embodiments the reactor further comprises a plurality of absorbers, provided that the exhaust outlet of one absorber is operatively connected to the exhaust inlet of another absorber. In some embodiments the exhaust comprises a gas comprising one or more of NOx, SOx, or COx. In some embodiments the gas comprises one or more of NOx, SOx, or COx. In some embodiments the reactor further comprises a plurality of absorbers, provided that all absorbers are operatively connected. In some embodiments the reactor further comprises a plurality of absorbers aligned in parallel, provided that each absorber comprises a liquid waste stream inlet, an exhaust inlet and an exhaust outlet, and provided that the liquid waste stream outlet of the biogas production unit is operatively connected to the liquid waste stream inlet of each absorber and the exhaust outlet of the device is operatively connected to the exhaust inlet of each absorber. In some embodiments the system is entirely modular (i.e. one or more reactors, absorbers, biogas production units, devices constructed to utilize the biogas stream to produce energy and an exhaust comprising a gas, fans, mist eliminators and/or other components are added or removed from the system). In some embodiments the reactor is entirely modular (i.e. one or more absorbers are interchangeable and/or additional absorbers are added to or removed from the reactor). In some embodiments the reactor comprises more than one absorber aligned in series, aligned in parallel, and/or aligned in a combination of in series and in parallel. In some embodiments the reactor comprises a first absorber and a second absorber aligned in parallel, provided that the first absorber and the second absorber are each operatively connected to the exhaust outlet of the biogas production unit. In some embodiments the reactor is rectangular or square. In some embodiments a liquid is anything that flows. In some embodiments, the device comprises a combustion engine, a boiler, a turbine or and/or a microturbine.

In one aspect of the systems, methods and devices described herein is a reactor comprising a first absorber comprising a first liquid waste stream inlet, a first exhaust inlet and a first exhaust outlet, the reactor operatively connected to: a biogas production unit constructed so as to convert waste to a biogas stream and a liquid waste stream and comprising a biogas stream outlet and a liquid waste stream outlet operatively connected to the first liquid waste stream inlet of the first absorber; and a device constructed to utilize the biogas stream to produce energy and an exhaust comprising a gas, the device comprising a biogas stream inlet operatively connected to the biogas stream outlet of the biogas production unit and an exhaust outlet operatively connected to the first exhaust inlet of the first absorber, provided that the reactor is constructed so as to treat exhaust so as to decrease or reduce emissions or pollutants by contacting the exhaust with the liquid waste stream from the biogas production unit. In some embodiments the exhaust comprises a gas comprising one or more of NOx, SOx, or COx. In some embodiments the gas comprises one or more of NOx, SOx, or COx. In some embodiments the reactor comprises more than one absorber aligned in series, aligned in parallel, and/or aligned in a combination of in series and in parallel. In some embodiments the reactor comprises a first absorber and a second absorber aligned in parallel, the first absorber comprising a first liquid waste stream inlet, a first exhaust inlet and a first exhaust outlet and the second absorber comprising a second liquid waste stream inlet, a second exhaust inlet and a second exhaust outlet, provided that the liquid waste stream outlet of the biogas production unit is operatively connected to the first liquid waste stream inlet of the first absorber and the second liquid waste stream inlet of the second absorber, the first exhaust inlet of the first absorber is operatively connected to the exhaust outlet of the biogas production unit, and the second exhaust inlet of the second absorber is operatively connected to the exhaust outlet of the biogas production unit. In some embodiments the a first reactor and a second reactor are aligned in series, provided that the first reactor is operatively connected to a fan, the first reactor comprising: a first absorber comprising a first liquid waste stream inlet, a first exhaust inlet and a first exhaust outlet; a second absorber comprising a second liquid waste stream inlet, a second exhaust inlet and a second exhaust outlet; a third absorber comprising a third liquid waste stream inlet; a third exhaust inlet and a third exhaust outlet; and a fourth absorber comprising a fourth liquid waste stream inlet, a fourth exhaust inlet and a fourth exhaust outlet, and the second reactor comprising: a fifth absorber comprising a fifth liquid waste stream inlet, a fifth exhaust inlet and a fifth exhaust outlet; a sixth absorber comprising a sixth liquid waste stream inlet, a sixth exhaust inlet and a sixth exhaust outlet; a seventh absorber comprising a seventh liquid waste stream inlet, a seventh exhaust inlet and a seventh exhaust outlet; and an eighth absorber comprising a eighth liquid waste stream inlet, an eighth exhaust inlet and an eighth exhaust outlet, provided that the liquid waste stream outlet of the biogas production unit is operatively connected to the first liquid waste stream inlet of the first absorber, the second liquid waste stream inlet of the second absorber, the third liquid waste stream inlet of the third absorber, the fourth liquid waste stream inlet of the fourth absorber, the fifth liquid waste stream inlet of the fifth absorber, the sixth liquid waste stream inlet of the sixth absorber, the seventh liquid waste stream inlet of the seventh absorber, and the eighth liquid waste stream inlet of the eighth absorber, and provided that the first exhaust inlet of the first absorber is operatively connected to the fan, the first exhaust outlet of the first absorber is operatively connected to the second exhaust inlet of the second absorber, the second exhaust outlet of the second absorber is operatively connected to the third exhaust inlet of the third absorber, the third exhaust outlet of the third absorber is operatively connected to the fourth exhaust inlet of the fourth absorber, the fifth exhaust inlet of the fifth absorber is operatively connected to the first reactor and/or the fourth exhaust outlet of the fourth absorber, the fifth exhaust outlet of the fifth absorber is operatively connected to the sixth exhaust inlet of the sixth absorber, the sixth exhaust outlet of the sixth absorber is operatively connected to the seventh exhaust inlet of the seventh absorber, and the seventh exhaust outlet of the seventh absorber is operatively connected to the eighth exhaust inlet of the eighth absorber. In some embodiments the reactor is rectangular or square. In some embodiments a liquid is anything that flows. In some embodiments, the device comprises a combustion engine, a boiler, a turbine or and/or a microturbine.

In one aspect of the systems, methods and devices described herein is a reactor comprising a first absorber comprising a first liquid waste stream inlet, a first exhaust inlet and a first exhaust outlet, the reactor operatively connected to: a biogas production unit constructed so as to convert waste to a biogas stream and a liquid waste stream and comprising a biogas stream outlet and a liquid waste stream outlet operatively connected to the first liquid waste stream inlet of the first absorber; and a device constructed to utilize the biogas stream to produce energy and an exhaust comprising a gas, the device comprising a biogas stream inlet operatively connected to the biogas stream outlet of the biogas production unit and an exhaust outlet operatively connected to the first exhaust inlet of the first absorber, provided that the reactor is constructed so as to treat exhaust so as to decrease or reduce emissions or pollutants by contacting the exhaust with the liquid waste stream from the biogas production unit. In some embodiments the exhaust comprises a gas comprising one or more of NOx, SOx, or COx. In some embodiments the gas comprises one or more of NOx, SOx, or COx. In some embodiments the reactor comprises more than one absorber aligned in series, aligned in parallel, and/or aligned in a combination of in series and in parallel. In some embodiments the a first reactor and a second reactor are aligned in parallel, each reactor operatively connected to a fan, the first reactor comprising: a first absorber comprising a first liquid waste stream inlet, a first exhaust inlet and a first exhaust outlet; a second absorber comprising a second liquid waste stream inlet, a second exhaust inlet and a second exhaust outlet; a third absorber comprising a third liquid waste stream inlet, a third exhaust inlet and a third exhaust outlet; and a fourth absorber comprising a fourth liquid waste stream inlet, a fourth exhaust inlet and a fourth exhaust outlet, and the second reactor comprising: a fifth absorber comprising a fifth liquid waste stream inlet, a fifth exhaust inlet and a fifth exhaust outlet; a sixth absorber comprising a sixth liquid waste stream inlet, a sixth exhaust inlet and a sixth exhaust outlet; a seventh absorber comprising a seventh liquid waste stream inlet, a seventh exhaust inlet and a seventh exhaust outlet; and an eighth absorber comprising an eighth liquid waste stream inlet, an eighth exhaust inlet and an eighth exhaust outlet, provided that the liquid waste stream outlet of the biogas production unit is operatively connected to the first liquid waste stream inlet of the first absorber, the second liquid waste stream inlet of the second absorber, the third liquid waste stream inlet of the third absorber, the fourth liquid waste stream inlet of the fourth absorber, the fifth liquid waste stream inlet of the fifth absorber, the sixth liquid waste stream inlet of the sixth absorber, the seventh liquid waste stream inlet of the seventh absorber, and the eighth liquid waste stream inlet of the eighth absorber, and provided that the first exhaust inlet of the first absorber is operatively connected to the fan, the first exhaust outlet of the first absorber is operatively connected to the second exhaust inlet of the second absorber, the second exhaust outlet of the second absorber is operatively connected to the third exhaust inlet of the third absorber, the third exhaust outlet of the third absorber is operatively connected to the fourth exhaust inlet of the fourth absorber, and provided that the fifth exhaust inlet of the fifth absorber is operatively connected to the fan, the fifth exhaust outlet of the fifth absorber is operatively connected to the sixth exhaust inlet of the sixth absorber, the sixth exhaust outlet of the sixth absorber is operatively connected to the seventh exhaust inlet of the seventh absorber, and the seventh exhaust outlet of the seventh absorber is operatively connected to the eighth exhaust inlet of the eighth absorber. In some embodiments the reactor is rectangular or square. In some embodiments a liquid is anything that flows. In some embodiments, the device comprises a combustion engine, a boiler, a turbine or and/or a microturbine.

In one aspect of the systems, methods and devices described herein is a method to treat exhaust comprising: providing a system comprising: a reactor comprising a first absorber comprising a first liquid waste stream inlet a first exhaust inlet, and a first exhaust outlet; a biogas production unit constructed so as to convert waste to a biogas stream and a liquid waste stream a liquid waste stream, provided that the liquid waste stream is operatively connected to the first liquid waste stream inlet of the first absorber; and a device constructed to utilize the biogas stream to produce energy and an exhaust comprising a gas, the device comprising a biogas stream inlet operatively connected to the biogas stream outlet of the biogas production unit and an exhaust outlet operatively connected to the first exhaust inlet of the first absorber; and contacting the exhaust with the liquid waste stream so as to treat exhaust so as to decrease or reduce emissions or pollutants. In some embodiments the reactor further comprises a second absorber comprising a second liquid waste stream inlet, a second exhaust inlet and a second exhaust outlet, provided that the liquid waste stream outlet of the biogas production unit is operatively connected to the first liquid waste stream inlet of the first absorber and the second liquid waste stream inlet of the second absorber, and provided that the exhaust outlet of the biogas production unit is operatively connected to the first exhaust inlet of the first absorber and the first exhaust outlet of the first absorber is operatively connected to the second exhaust inlet of the second absorber. In some embodiments the reactor further comprises a second absorber comprising a second liquid waste stream inlet, a second exhaust inlet and a second exhaust outlet and a third absorber comprising a third liquid waste stream inlet, a third exhaust inlet and a third exhaust outlet, provided that the liquid waste stream outlet of the biogas production unit is operatively connected to the first liquid waste stream inlet of the first absorber, the second liquid waste stream inlet of the second absorber, and the third liquid waste stream inlet of the third absorber, and provided that the exhaust outlet of the biogas production unit is operatively connected to the first exhaust inlet of the first absorber, the first exhaust outlet of the first absorber is operatively connected to the second exhaust inlet of the second absorber, and the second exhaust outlet of the second absorber is operatively connected to third the exhaust inlet of the third absorber. In some embodiments the reactor further comprises a second absorber comprising a second liquid waste stream inlet, a second exhaust inlet and a second exhaust outlet, a third absorber comprising a third liquid waste stream inlet, a third exhaust inlet and a third exhaust outlet and a fourth absorber comprising a fourth liquid waste stream inlet, a fourth exhaust inlet and a fourth exhaust outlet, provided that the liquid waste stream outlet of the biogas production unit is operatively connected to the first liquid waste stream inlet of the first absorber, the second liquid waste stream inlet of the second absorber, the third liquid waste stream inlet of the third absorber, and the fourth liquid waste stream inlet of the fourth absorber, and provided that the exhaust outlet of the biogas production unit is operatively connected to the first exhaust inlet of the first absorber, the first exhaust outlet of the first absorber is operatively connected to the second exhaust inlet of the second absorber, the second exhaust outlet of the second absorber is operatively connected to third the exhaust inlet of the third absorber, and the third exhaust outlet of the third absorber is operatively connected to fourth the exhaust inlet of the fourth absorber. In some embodiments the reactor further comprises a plurality of absorbers, provided that the exhaust outlet of one absorber is operatively connected to the exhaust inlet of another absorber. In some embodiments the exhaust comprises a gas comprising one or more of NOx, SOx, or COx. In some embodiments the gas comprises one or more of NOx, SOx, or COx. In some embodiments the reactor further comprises a plurality of absorbers, provided that all absorbers are operatively connected. In some embodiments the reactor further comprises a plurality of absorbers aligned in parallel, provided that each absorber comprises a liquid waste stream inlet, an exhaust inlet and an exhaust outlet, and provided that the liquid waste stream outlet of the biogas production unit is operatively connected to the liquid waste stream inlet of each absorber and the exhaust outlet of the device is operatively connected to the exhaust inlet of each absorber. In some embodiments the system is entirely modular (i.e. one or more reactors, absorbers, biogas production units, devices constructed to utilize the biogas stream to produce energy and an exhaust comprising a gas, fans, mist eliminators and/or other components are added or removed from the system). In some embodiments the reactor is entirely modular (i.e. one or more absorbers are interchangeable and/or additional absorbers are added to or removed from the reactor). In some embodiments the reactor comprises more than one absorber aligned in series, aligned in parallel, and/or aligned in a combination of in series and in parallel. In some embodiments the reactor comprises a first absorber and a second absorber aligned in parallel, provided that the first absorber and the second absorber are each operatively connected to the exhaust outlet of the biogas production unit. In some embodiments the reactor is rectangular or square. In some embodiments a liquid is anything that flows. In some embodiments, the device comprises a combustion engine, a boiler, a turbine or and/or a microturbine.

In one aspect of the systems, methods and devices described herein is a method to treat exhaust comprising: providing a system comprising: a reactor comprising a first absorber comprising a first liquid waste stream inlet a first exhaust inlet, and a first exhaust outlet; a biogas production unit constructed so as to convert waste to a biogas stream and a liquid waste stream a liquid waste stream, provided that the liquid waste stream is operatively connected to the first liquid waste stream inlet of the first absorber; and a device constructed to utilize the biogas stream to produce energy and an exhaust comprising a gas, the device comprising a biogas stream inlet operatively connected to the biogas stream outlet of the biogas production unit and an exhaust outlet operatively connected to the first exhaust inlet of the first absorber; and contacting the exhaust with the liquid waste stream so as to treat exhaust so as to decrease or reduce emissions or pollutants. In some embodiments the exhaust comprises a gas comprising one or more of NOx, SOx, or COx. In some embodiments the gas comprises one or more of NOx, SOx, or COx. In some embodiments the reactor comprises more than one absorber aligned in series, aligned in parallel, and/or aligned in a combination of in series and in parallel. In some embodiments the reactor comprises a first absorber and a second absorber aligned in parallel, the first absorber comprising a first liquid waste stream inlet, a first exhaust inlet and a first exhaust outlet and the second absorber comprising a second liquid waste stream inlet, a second exhaust inlet and a second exhaust outlet, provided that the liquid waste stream outlet of the biogas production unit is operatively connected to the first liquid waste stream inlet of the first absorber and the second liquid waste stream inlet of the second absorber, the first exhaust inlet of the first absorber is operatively connected to the exhaust outlet of the biogas production unit, and the second exhaust inlet of the second absorber is operatively connected to the exhaust outlet of the biogas production unit. In some embodiments the system comprises a fan, a first reactor and a second reactor, the first and second reactors aligned in series, the first reactor comprising: a first absorber comprising a first liquid waste stream inlet, a first exhaust inlet and a first exhaust outlet; a second absorber comprising a second liquid waste stream inlet, a second exhaust inlet and a second exhaust outlet; a third absorber comprising a third liquid waste stream inlet, a third exhaust inlet and a third exhaust outlet; and a fourth absorber comprising a fourth liquid waste stream inlet, a fourth exhaust inlet and a fourth exhaust outlet, and the second reactor comprising: a fifth absorber comprising a fifth liquid waste stream inlet, a fifth exhaust inlet and a fifth exhaust outlet; a sixth absorber comprising a sixth liquid waste stream inlet, a sixth exhaust inlet and a sixth exhaust outlet; a seventh absorber comprising a seventh liquid waste stream inlet, a seventh exhaust inlet and a seventh exhaust outlet; and an eighth absorber comprising a eighth liquid waste stream inlet, an eighth exhaust inlet and an eighth exhaust outlet, provided that the liquid waste stream outlet of the biogas production unit is operatively connected to the first liquid waste stream inlet of the first absorber, the second liquid waste stream inlet of the second absorber, the third liquid waste stream inlet of the third absorber, the fourth liquid waste stream inlet of the fourth absorber, the fifth liquid waste stream inlet of the fifth absorber, the sixth liquid waste stream inlet of the sixth absorber, the seventh liquid waste stream inlet of the seventh absorber, and the eighth liquid waste stream inlet of the eighth absorber, and provided that the first exhaust inlet of the first absorber is operatively connected to the fan, the first exhaust outlet of the first absorber is operatively connected to the second exhaust inlet of the second absorber, the second exhaust outlet of the second absorber is operatively connected to the third exhaust inlet of the third absorber, the third exhaust outlet of the third absorber is operatively connected to the fourth exhaust inlet of the fourth absorber, the fifth exhaust inlet of the fifth absorber is operatively connected to the first reactor and/or the fourth exhaust outlet of the fourth absorber, the fifth exhaust outlet of the fifth absorber is operatively connected to the sixth exhaust inlet of the sixth absorber, the sixth exhaust outlet of the sixth absorber is operatively connected to the seventh exhaust inlet of the seventh absorber, and the seventh exhaust outlet of the seventh absorber is operatively connected to the eighth exhaust inlet of the eighth absorber. In some embodiments the reactor is rectangular or square. In some embodiments a liquid is anything that flows. In some embodiments, the device comprises a combustion engine, a boiler, a turbine or and/or a microturbine.

In one aspect of the systems, methods and devices described herein is a method to treat exhaust comprising: providing a system comprising: a reactor comprising a first absorber comprising a first liquid waste stream inlet a first exhaust inlet, and a first exhaust outlet; a biogas production unit constructed so as to convert waste to a biogas stream and a liquid waste stream a liquid waste stream, provided that the liquid waste stream is operatively connected to the first liquid waste stream inlet of the first absorber; and a device constructed to utilize the biogas stream to produce energy and an exhaust comprising a gas, the device comprising a biogas stream inlet operatively connected to the biogas stream outlet of the biogas production unit and an exhaust outlet operatively connected to the first exhaust inlet of the first absorber; and contacting the exhaust with the liquid waste stream so as to treat exhaust so as to decrease or reduce emissions or pollutants. In some embodiments the exhaust comprises a gas comprising one or more of NOx, SOx, or COx. In some embodiments the gas comprises one or more of NOx, SOx, or COx. In some embodiments the reactor comprises more than one absorber aligned in series, aligned in parallel, and/or aligned in a combination of in series and in parallel. In some embodiments the system comprises a fan, a first reactor and a second reactor, the first and second reactors aligned in parallel, the first reactor comprising: a first absorber comprising a first liquid waste stream inlet, a first exhaust inlet and a first exhaust outlet; a second absorber comprising a second liquid waste stream inlet, a second exhaust inlet and a second exhaust outlet; a third absorber comprising a third liquid waste stream inlet, a third exhaust inlet and a third exhaust outlet; and a fourth absorber comprising a fourth liquid waste stream inlet, a fourth exhaust inlet and a fourth exhaust outlet, and the second reactor comprising: a fifth absorber comprising a fifth liquid waste stream inlet, a fifth exhaust inlet and a fifth exhaust outlet; a sixth absorber comprising a sixth liquid waste stream inlet, a sixth exhaust inlet and a sixth exhaust outlet; a seventh absorber comprising a seventh liquid waste stream inlet, a seventh exhaust inlet and a seventh exhaust outlet; and an eighth absorber comprising an eighth liquid waste stream inlet, an eighth exhaust inlet and an eighth exhaust outlet, provided that the liquid waste stream outlet of the biogas production unit is operatively connected to the first liquid waste stream inlet of the first absorber, the second liquid waste stream inlet of the second absorber, the third liquid waste stream inlet of the third absorber, the fourth liquid waste stream inlet of the fourth absorber, the fifth liquid waste stream inlet of the fifth absorber, the sixth liquid waste stream inlet of the sixth absorber, the seventh liquid waste stream inlet of the seventh absorber, and the eighth liquid waste stream inlet of the eighth absorber, and provided that the first exhaust inlet of the first absorber is operatively connected to the fan, the first exhaust outlet of the first absorber is operatively connected to the second exhaust inlet of the second absorber, the second exhaust outlet of the second absorber is operatively connected to the third exhaust inlet of the third absorber, the third exhaust outlet of the third absorber is operatively connected to the fourth exhaust inlet of the fourth absorber, and provided that the fifth exhaust inlet of the fifth absorber is operatively connected to the fan, the fifth exhaust outlet of the fifth absorber is operatively connected to the sixth exhaust inlet of the sixth absorber, the sixth exhaust outlet of the sixth absorber is operatively connected to the seventh exhaust inlet of the seventh absorber, and the seventh exhaust outlet of the seventh absorber is operatively connected to the eighth exhaust inlet of the eighth absorber. In some embodiments the reactor is rectangular or square. In some embodiments a liquid is anything that flows. In some embodiments, the device comprises a combustion engine, a boiler, a turbine or and/or a microturbine.

In certain embodiments, the subject matter described herein incorporates a modular multi-stage wet-scrubbing system that utilizes wastewater from the anaerobic digester with a NOx oxidation strategy. The multiple absorbers, in certain embodiments, are arranged in series or in parallel. The first stage of the system is the Quencher/Cooler, which simultaneously cools and saturates the exhaust with water. In one embodiment, after the Quencher/Cooler the exhaust has been cooled to approximately 100-120° F. (or lower), it is suitably conditioned for rapid and selective NO oxidation.

In some embodiments of the systems, methods and devices described herein, a modular multi-stage wet-scrubbing system that utilizes wastewater from the anaerobic digester with a NOx oxidation strategy is incorporated. The multiple absorbers, in certain embodiments, are arranged in series or in parallel. Optionally, a first stage of the system is a quenching stage, wherein a quencher saturates exhaust with water and cools the exhaust to about 90° F., about 100° F., about 110° F., about 120° F., about 130° F., about 140° F., or about 150° F. Once the exhaust has been cooled it is suitably conditioned for rapid and selective NO oxidation. Optionally a cooler is included provided that the cooler is operatively connected to the quencher, and provided that the cooler further aids in cooling the exhaust. The cooler and/or the quencher are not necessarily included for proper function of the systems, methods and devices described herein.

In one embodiment, following NO oxidation the exhaust enters an absorber array. Within the first absorber, a quantity of the NOx is removed because the NO₂ absorbs and reacts within the absorber with the anaerobic digester effluent. The NOx level in the exhaust stream is similarly reduced by each subsequent absorber stage before leaving the stack containing the targeted levels of SOx and NOx emissions.

In some embodiments of the systems, methods and devices described herein two absorbers are provided. In some embodiments, the first absorber is about one foot, about two feet, about three feet, or about four feet in diameter and contains polypropylene packing media, including as one specific example, at least about 10 vertical feet, about 11, about 12, about 13.5. about 15 or about 20 vertical feet of 2.3″ LANPAC® polypropylene packed media, and the second absorber is of the same or different diameter and contains polypropylene media, including for example at least about 5, about 6, about 7, about 8, about 9, or about 10, about 11, about 12, about 13, about 15 or about 20 vertical feet of 2.3″ LANPAC® polypropylene packed media. In some embodiments, the first absorber is rectangular or square having a cross-sectional width of about one foot, about two feet, about three feet, or about four feet and contains polypropylene packing media, including as one specific example, at least about 10 vertical feet, about 11, about 12, about 13.5. about 15 or about 20 vertical feet of 2.3″ LANPAC® polypropylene packed media, and the second absorber is rectangular or square having a cross-sectional width of the same or different dimension and contains polypropylene media, including for example at least about 5, about 6, about 7, about 8, about 9, or about 10, about 11, about 12, about 13, about 15 or about 20 vertical feet of 2.3″ LANPAC® polypropylene packed media. Other types of packed media, in certain embodiments, is used including ceramic saddles, and larger LANPAC-XL® polypropylene media each with varying effective surface area, void fraction and pressure drop. In some embodiments, the AD effluent is introduced into the top of each absorber at a rate of about 3-20 gallons per minute per square foot of cross sectional surface area, and about 20-85% of the NO₂ is absorbed into and reacted with the AD effluent at each stage. In certain embodiments, the AD effluent is introduced into the top of each absorber at a rate of about 5-18 gallons per minute per square foot of cross sectional surface area and about 50-75% of the NO₂ is absorbed into and reacted with the AD effluent at each stage. In certain embodiments, the AD effluent is introduced into the top of each absorber at a rate of about 7-15 gallons per minute per square foot of cross sectional surface area and at least about 25% of the NO₂ is absorbed into and reacted with the AD effluent at each stage. In certain embodiments, the AD effluent is introduced into the top of each absorber at a rate of about 9-12 gallons per minute per square foot of cross sectional surface area and at least about 35% of the NO₂ is absorbed into and reacted with the AD effluent at each stage. In some embodiments, a combination of circular and rectangular absorbers are used.

In some embodiments of the systems, methods and devices described herein three absorbers are provided. In some embodiments, the first absorber is about one foot, about two feet, about three feet, or about four feet in diameter and contains polypropylene packing media, including as one specific example, at least about 10 vertical feet, about 11, about 12, about 13.5. about 15 or about 20 vertical feet of 2.3″ LANPAC® polypropylene packed media, and the second and third absorbers are of the same or different diameter and contain polypropylene media, including for example at least about 5, about 6, about 7, about 8, about 9, or about 10, about 11, about 12, about 13, about 15 or about 20 vertical feet of 2.3″ LANPAC® polypropylene packed media. In some embodiments, the first absorber is rectangular or square having a cross-sectional width of about one foot, about two feet, about three feet, or about four feet and contains polypropylene packing media, including as one specific example, at least about 10 vertical feet, about 11, about 12, about 13.5. about 15 or about 20 vertical feet of 2.3″ LANPAC® polypropylene packed media, and the second and third absorbers are rectangular or square having a cross-sectional width of the same or different dimension and contains polypropylene media, including for example at least about 5, about 6, about 7, about 8, about 9, or about 10, about 11, about 12, about 13, about 15 or about 20 vertical feet of 2.3″ LANPAC® polypropylene packed media. Other types of packed media, in certain embodiments, is used including ceramic saddles, and larger LANPAC-XL® polypropylene media each with varying effective surface area, void fraction and pressure drop. In some embodiments, the AD effluent is introduced into the top of each absorber at a rate of about 3-20 gallons per minute per square foot of cross sectional surface area, and about 20-85% of the NO₂ is absorbed into and reacted with the AD effluent at each stage. In certain embodiments, the AD effluent is introduced into the top of each absorber at a rate of about 5-18 gallons per minute per square foot of cross sectional surface area and about 50-75% of the NO₂ is absorbed into and reacted with the AD effluent at each stage. In certain embodiments, the AD effluent is introduced into the top of each absorber at a rate of about 7-15 gallons per minute per square foot of cross sectional surface area and at least about 25% of the NO₂ is absorbed into and reacted with the AD effluent at each stage. In certain embodiments, the AD effluent is introduced into the top of each absorber at a rate of about 9-12 gallons per minute per square foot of cross sectional surface area and at least about 35% of the NO₂ is absorbed into and reacted with the AD effluent at each stage. In some embodiments, a combination of circular and rectangular absorbers are used.

In some embodiments of the systems, methods and devices described herein four absorbers are provided. In some embodiments, the first absorber is about one foot, about two feet, about three feet, or about four feet in diameter and contains polypropylene packing media, including as one specific example, at least about 10 vertical feet, about 11, about 12, about 13.5. about 15 or about 20 vertical feet of 2.3″ LANPAC® polypropylene packed media, and the second, third and fourth absorbers are of the same or different diameter and contain polypropylene media, including for example at least about 5, about 6, about 7, about 8, about 9, or about 10, about 11, about 12, about 13, about 15 or about 20 vertical feet of 2.3″ LANPAC® polypropylene packed media. In some embodiments, the first absorber is rectangular or square having a cross-sectional width of about one foot, about two feet, about three feet, or about four feet and contains polypropylene packing media, including as one specific example, at least about 10 vertical feet, about 11, about 12, about 13.5. about 15 or about 20 vertical feet of 2.3″ LANPAC® polypropylene packed media, and the second, third and fourth absorbers are rectangular or square having a cross-sectional width of the same or different dimension and contains polypropylene media, including for example at least about 5, about 6, about 7, about 8, about 9, or about 10, about 11, about 12, about 13, about 15 or about 20 vertical feet of 2.3″ LANPAC® polypropylene packed media. Other types of packed media, in certain embodiments, is used including ceramic saddles, and larger LANPAC-XL® polypropylene media each with varying effective surface area, void fraction and pressure drop. In some embodiments, the AD effluent is introduced into the top of each absorber at a rate of about 3-20 gallons per minute per square foot of cross sectional surface area, and about 20-85% of the NO₂ is absorbed into and reacted with the AD effluent at each stage. In certain embodiments, the AD effluent is introduced into the top of each absorber at a rate of about 5-18 gallons per minute per square foot of cross sectional surface area and about 50-75% of the NO₂ is absorbed into and reacted with the AD effluent at each stage. In certain embodiments, the AD effluent is introduced into the top of each absorber at a rate of about 7-15 gallons per minute per square foot of cross sectional surface area and at least about 25% of the NO₂ is absorbed into and reacted with the AD effluent at each stage. In certain embodiments, the AD effluent is introduced into the top of each absorber at a rate of about 9-12 gallons per minute per square foot of cross sectional surface area and at least about 35% of the NO₂ is absorbed into and reacted with the AD effluent at each stage. In some embodiments, a combination of circular and rectangular absorbers are used.

In some embodiments of the systems, methods and devices described herein a plurality absorbers are provided. In some embodiments, each absorber is about one foot, about two feet, about three feet, or about four feet in diameter and contains polypropylene packing media, including as one specific example, at least about 5, about 6, about 7, about 8, about 9, about 10, about 11, about 12, or about 13.5, about 15, or about 20 vertical feet of 2.3″ LANPAC® polypropylene packed media. In some embodiments, each absorber is rectangular or square and has a cross-sectional width of about one foot, about two feet, about three feet, or about four feet and contains polypropylene packing media, including as one specific example, at least about 5, about 6, about 7, about 8, about 9, about 10, about 11, about 12, or about 13.5, about 15, or about 20 vertical feet of 2.3″ LANPAC® polypropylene packed media. Other types of packed media, in certain embodiments, is used including ceramic saddles, and larger LANPAC-XL® polypropylene media each with varying effective surface area, void fraction and pressure drop. In some embodiments, the AD effluent is introduced into the top of each absorber at a rate of about 3-20 gallons per minute per square foot of cross sectional surface area, and about 20-85% of the NO₂ is absorbed into and reacted with the AD effluent at each stage. In certain embodiments, the AD effluent is introduced into the top of each absorber at a rate of about 5-18 gallons per minute per square foot of cross sectional surface area and about 50-75% of the NO₂ is absorbed into and reacted with the AD effluent at each stage. In certain embodiments, the AD effluent is introduced into the top of each absorber at a rate of about 7-15 gallons per minute per square foot of cross sectional surface area and at least about 25% of the NO₂ is absorbed into and reacted with the AD effluent at each stage. In certain embodiments, the AD effluent is introduced into the top of each absorber at a rate of about 9-12 gallons per minute per square foot of cross sectional surface area and at least about 35% of the NO₂ is absorbed into and reacted with the AD effluent at each stage. In some embodiments, a combination of circular and rectangular absorbers are used.

### Waste

The waste in the biogas production unit in certain embodiments is agricultural waste, animal waste, municipal waste, sewage, or waste from a landfill.

In some embodiments, the agricultural waste comprises feces or urine. In some embodiments, the animal waste is manure. Most animal manure is feces. Common forms of animal manure include farmyard manure (FYM) or farm slurry (liquid manure). FYM also contains plant material (example, straw), which has been used as bedding for animals and has absorbed the feces and urine. Agricultural manure in liquid form, known as slurry, is produced by more intensive livestock rearing systems where concrete or slats are used, instead of straw bedding.

In some embodiments, the waste is municipal waste. Municipal waste results at least in part from anthropogenic effects, processes or materials that are derived at least in part from human activities. In certain embodiments, the municipal waste comprises wastewater. Wastewater comprises any water that has been adversely affected in quality by anthropogenic influence. In certain embodiments, wastewater comprises liquid waste discharged by domestic residences, commercial properties, industry, and/or agriculture and encompass a wide range of potential contaminants and concentrations. In the most common usage, it refers to the municipal wastewater that contains a broad spectrum of contaminants resulting from the mixing of wastewaters from different sources. In further or additional embodiments, the waste is sewage. Sewage is a subset of wastewater that is contaminated with feces or urine. As used herein, “sewage” includes domestic, municipal, or industrial liquid waste products disposed of, usually via a pipe or sewer or similar structure including for example a cesspool emptier. In still further or additional embodiments, the waste comprises an organic material, including for example waste from food processing facilities, paper mills, meat processing facilities, and the like.

In some embodiments, the waste is landfill waste. In specific embodiments, the waste from a landfill comprises leachate. In further or additional embodiments, the landfill waste is piped into the systems described herein from the landfill.

### Microorganisms in Absorbers

In some embodiments the system comprises (a) an absorber that comprises a microorganism that is capable of reducing a nitrogenous compound, provided that the absorber is operatively connected to a liquid source and an engine exhaust source; and (b) an engine that produces energy and NOx-containing engine exhaust.

In some embodiments, the absorption of NO₂ into, and subsequent reaction with water produces nitrate ions. Nitrate reducing bacteria are known anaerobes and aerobes expected to be present within the vast bacterial consortium present in the anaerobic digester effluent. These microbes are capable of reducing nitrate to nitrogen gas. In some embodiments, the NOx is reduced by a microorganism when engine exhaust is contacted with a liquid waste stream in the absorber. The microorganism in certain embodiments is bacteria. The bacteria, in certain embodiments, comprises a nitrogen (nitrate?) reducing bacteria. Some illustrative but not limiting examples of nitrogen (nitrate, de-nitrification?) reducing bacteria are Thiobacillus denitrificans, Micrococcus denitrificans, Paracoccus denitrificans, and Pseudomonas. In some embodiments, the bacteria comprises sulfate-reducing bacteria. The microbes in the absorber are used in conjunction with the chemical cocktail found in the effluent. The microbes in certain embodiments have a short contact time with the biogas exhaust within the absorber.

In some embodiments, the engine is a combustion engine. In certain embodiments, the engine is a combustion engine, such as a biogas engine, internal combustion engine, turbine or boiler. In some embodiments, the device is a combustion engine, a biogas engine, internal combustion engine, turbine, boiler, or gas fired boiler

In some embodiments, the system comprises a biogas production unit that converts waste to a biogas stream and a liquid waste stream. The biogas production unit provides liquid waste to the absorber and biogas to the combustion engine. In some embodiments, the system is a wet scrubbing system.

In some embodiments, NOx is reduced by bacteria when engine exhaust is contacted with a liquid waste stream in the absorber. Some embodiments further comprise a quencher that is operatively connected to an outlet from the combustion engine. Some embodiments comprise a cooler that is operatively connected to the quencher. Some embodiments comprise more than one absorber arranged in series and/or in parallel.

In some embodiments, the biogas production unit is an anaerobic digester. In some embodiments, the waste is agricultural waste or municipal waste. In further or additional embodiments, the municipal waste comprises wastewater.

### Fan

In some embodiments, the system described herein provides a method of decreasing NOx emissions in an engine exhaust comprising contacting the engine exhaust from the combustion engine with the liquid waste stream from the biogas production unit to generate a treated exhaust with decreased NOx emissions. In some embodiments, the system comprises (a) an absorber that is operatively connected to a liquid source and an engine exhaust source; (b) an engine that produces energy and engine exhaust containing NOx; and (c) a fan that is located within the system. The fan, in certain embodiments, reduces back pressure on the engine. The fan or blower, in certain applications, impacts ozone conservation and reaction specificity. The fan or blower is not necessarily included for proper function of the systems, methods and devices described herein.

In some embodiments, the fan is operatively connected to a quencher, a cooler, the absorber, or a combination thereof. In some embodiments, the fan is operatively connected to a cooler and the absorber. In some embodiments, provided is more than one absorber arranged in series and/or in parallel. In some embodiments, provided is one or more fans operatively connected to one or more absorbers.

In some embodiments, placement of the fan between the Cooler and the Absorber is preferable and superior to placement after the absorber. For example, single absorber performance has been shown to increase at least about 5%, at least about 6%, at least about 7%, at least about 8%, at least about 9%, or at least about 10% by moving the exhaust fan from after the absorber to between the cooler and the absorber. This increased absorption efficiency, in certain embodiments, is a result of the slight positive pressure that results from pushing the exhaust through the absorber rather than the negative pressure resulting from pulling it through.

In some embodiments, provided are multiple absorbers arranged in series or in parallel successively. In some embodiments, the biogas production unit in the system described herein is an anaerobic digester. The waste in the anaerobic digester, in certain embodiments, is agricultural waste or municipal waste. The municipal waste, in certain embodiments, comprises sewage wastewater or solid waste from a landfill or a combination of the two.

### Reactive Chemical Agents Used in Reactor

The inventors have discovered that the digester wastewater contains a mixture of components that act, potentially in concert, to remove NOx from engine exhaust. The inventors furthered discovered that the difficulty in absorbing low concentrations of NOx (<500 ppm) at temperatures below 100° F., where no appreciable difference has been reported between 20% NaOH and pure water, supports this assertion. Furthermore, due to the constant flow design of most anaerobic digesters an essentially unlimited and constantly renewable supply of these scrubbing chemicals is already on site within the digester effluent.

Hydrogen sulfide (H₂S) is a product of anaerobic digestion and exists in relatively high concentrations in both the waste water effluent and biogas from digesters. The H₂S present in biogas is oxidized to SO₂ upon combustion. The alkaline solutions in the absorber dissolve SO₂ to yield sulfite. In some embodiments, the sulfite solution is used to destroy NOx through the following equations.

2Na₂SO₃+2NO→2Na₂SO₄+N₂

4Na₂SO₃+2NO₂→4Na₂SO₄+N₂

In some embodiments, aqueous solutions of sodium carbonate, calcium carbonate or ammonia are used for the absorption process. These substances, in some embodiments, exist in the digester wastewater; the digester effluent contains a useful mixture of valuable scrubbing agents and because of this requires no exogenous reactants.

Furthermore, the oxidation-reduction potential (ORP) is a measurement of the oxidizing or reducing ability of a solution. The presence of chemical reducing agents within a solution decreases the solution's ORP value. Methane production within an AD facility typically occurs at an ORP value between −175 mV and −400 mV and fresh AD effluent typically measures within this range. This highly reducing effluent is beneficial to overall NOx absorption and reduction from engine exhaust. The digester effluent contains a useful mixture of valuable scrubbing agents and because of this the method requires no exogenous reactants.

In some embodiments, urea, the primary metabolite of all mammalian protein metabolism, and a major constituent of urine, is present in the digester liquid waste. Urea promotes NOx absorption and breakdown. In some embodiments, a saddle-packed scrubber is used to irreversibly absorb NO and NO₂ into an aqueous urea solution to produce nitric and nitrous acid which both react further to the fertilizer ammonium nitrate.

### Metal and Non-Metal Catalysis Technology

In some embodiments, the multistage wet scrubbing system comprises a liquid waste stream inlet, an engine exhaust inlet, a quencher, cooler and an absorber, provided that the system is connected to (a) a biogas production unit that converts waste to a biogas stream and a liquid waste stream; and (b) a combustion engine that utilizes the biogas stream to produce energy and an engine exhaust stream containing NOx and comprising a biogas stream inlet and an engine exhaust stream outlet connected to the engine exhaust inlet of an absorber further provided that a metal or non-metal catalyst is contacted with the engine exhaust. In some embodiments, the catalyst is a rare-earth metal or zeolite or transition metal. In some embodiments, the engine exhaust is at a temperature of about 300° F., 350° F., 400° F., 450° F., 500° F., 550° F., 600° F., or 650° F. with temperature ranges below and above.

In some embodiments, the system described herein enables a biogas project to use a low cost and highly efficient internal combustion engine to produce both electrical and thermal energy and is superior to other applications which are optionally used in combination with the subject matter described herein, including fuel cells, micro-turbines, upgrading biogas for the pipeline, or gasification.

### Oxidants

In some embodiments, the system described herein provides a method of decreasing NOx emissions in an engine exhaust comprising contacting the engine exhaust from the combustion engine with the liquid waste stream from the biogas production unit to generate a treated exhaust with decreased NOx emissions. The biogas production unit, in certain embodiments, comprises an anaerobic digester that converts waste to a biogas stream.

In some embodiments, the system comprises (a) an absorber that is operatively connected to an engine exhaust source; (b) a combustion engine that produces energy and engine exhaust containing NOx; and (c) an oxidant source. In some embodiments, a biogas production unit converts waste to a biogas stream and a liquid waste stream, and a combustion engine utilizes the biogas stream to produce energy and an engine exhaust stream containing NOx whereby the engine exhaust gas stream is treated with an oxidant. In some embodiments, the oxidant source is ozone or hydrogen peroxide. In some embodiments an oxidation catalyst is used. Oxidation catalysts are commercially available from the following sources: BASF Corporation; Sud Chemie; or Johnson Matthey. In some embodiments, the oxidant is introduced to the exhaust prior to the exhaust entering an absorber.

In some embodiments, oxidative catalysts include γAl₂O₃, TiO₂, silica gel, MnO, Cr₂O₃, Co₃O₄ supported on γ Al₂O₃, Pt/Al₂O₃, MnO and Cr₂O₃ on the support of TiO₂, Pt/TiO₂, Cr/TiO₂, Cu/TiO₂, MnOx/TiO₂, Ce(x)Mn(y)Ti, AuCuPd/SiO₂. In some embodiments oxidative catalysts include base metals: Pt, Au, Ru, Rh, Ir, and Pd; support types: Al2O3, MgO, CeO2, CuO, TiO2, ZnO, MnO, FeO, Nb2O5, SiO2, ZrO2, La2O3, Co3O4, and Zeolite activated carbon; and promoter types: Ce, Co, Mg, Fe, Cu, Mn, Zr, K, Ni, Sn, Li, Na, Rb, Cs, Nb, La, Ba, Pb, Sm, and Zn. In some embodiments, oxidative catalysts comprise photocatalytic oxidation, for example UV irradiation (<387 nm) and a photocatalyst comprising, for example, SrTiO₃, TiO₂, ZnO, ZnS, CdS, and TiO₂. In some embodiments, oxidative catalysts comprise photocatalytic oxidation, for example UV irradiation and a photocatalyst comprising, for example, TiO₂ modified with Pt or Zn, which in some embodiments extends the wavelength activity range.

In some embodiments, oxidative catalysts for NO oxidation comprise γAl₂O₃, TiO₂, silica gel, MnO and Cr₂O₃ on the support of TiO₂, Pt/TiO₂, Cr/TiO₂, and Cu/TiO₂. In some embodiments, oxidative catalysts for selective oxidation of NO with O₂ comprise MnOx/TiO₂ and Ce(x)Mn(y)Ti. In some embodiments, oxidative catalysts for CO oxidation comprise AuCuPd/SiO₂. In some embodiments, oxidative catalysts for CO oxidation comprise base metals: Pt, Au, Ru, Rh, Ir, and Pd; support types: Al2O3, MgO, CeO2, CuO, TiO2, ZnO, MnO, FeO, Nb2O5, SiO2, ZrO2, La2O3, Co3O4, and Zeolite activated carbon; and promoter types: Ce, Co, Mg, Fe, Cu, Mn, Zr, K, Ni, Sn, Li, Na, Rb, Cs, Nb, La, Ba, Pb, Sm, and Zn.

In some embodiments, oxidative catalysts for CO oxidation comprise photocatalytic oxidation, for example UV irradiation (<387 nm) and a photocatalyst comprising, for example, SrTiO₃, TiO₂, ZnO, ZnS, CdS, and TiO₂. In some embodiments, oxidative catalysts for CO oxidation comprise photocatalytic oxidation, for example UV irradiation and a photocatalyst comprising, for example, TiO₂ modified with Pt or Zn, which in some embodiments extends wavelength activity range.

In some embodiments, provided is more than one oxidant, or a combination of oxidants and oxidation catalysts. The oxidant is operatively connected to the engine exhaust, so that the engine exhaust is pre-treated with the oxidant before entry into each absorber. The system, in certain embodiments, comprises more than one absorber and an oxidant source is operatively connected to each absorber. The system, in certain embodiments, comprises one or more inlets for the oxidant source post cooler. The oxidant, in further or additional embodiments, is ozone or hydrogen peroxide. In some embodiments the oxidant is EnviCat from the manufacturer Sud-Chemie. In some embodiments, the oxidant is Camet and/or CatCO from the manufacturer BASF.

In some embodiments, if the oxidant is H₂O₂, infusion occurs into the hot exhaust, prior to the Cooler and Quencher assembly.

In some embodiments, the oxidant source in the system is a single point, continuous infusion of ozone into the engine exhaust In some embodiments, the single point comprises a single location with a single point of infusion. In some embodiments, the single point comprises a single location with multiple points of infusion. In some embodiments, the single point comprises multiple injection points. In some embodiments, the single point comprises a single location with a single inlet. In some embodiments, the single point comprises a single location with multiple inlets. In some embodiments, the single point comprises multiple inlets. In some embodiments, the single point comprises one or more locations around a 360 degree circumference a duct, pipe, tube, or other means of delivering gas and/or liquid from one location to another. In some embodiments, the single point comprises a single location of a duct, pipe, tube, or other means of delivering gas and/or liquid from one location to another with two injection points separated by 180 degrees. In some embodiments, the single point comprises a single location of a duct, pipe, tube, or other means of delivering gas and/or liquid from one location to another with four injection points each separated by 90 degrees. In some embodiments, less than a stoichiometric mole of ozone is provided compared to NO (mol: mol). In some embodiments, sub-stoichiometric, continuous infusions of ozone occur at one or more locations along an absorption array.

In some embodiments, the oxidant source in the system is a single point, continuous infusion of H₂O₂ into the engine exhaust. In some embodiments, the single point comprises a single location with a single point of infusion. In some embodiments, the single point comprises a single location with multiple points of infusion. In some embodiments, the single point comprises multiple injection points. In some embodiments, the single point comprises a single location with a single inlet. In some embodiments, the single point comprises a single location with multiple inlets. In some embodiments, the single point comprises multiple inlets. In some embodiments, the single point comprises one or more locations around a 360 degree circumference a duct, pipe, tube, or other means of delivering gas and/or liquid from one location to another. In some embodiments, the single point comprises a single location of a duct, pipe, tube, or other means of delivering gas and/or liquid from one location to another with two injection points separated by 180 degrees. In some embodiments, the single point comprises a single location of a duct, pipe, tube, or other means of delivering gas and/or liquid from one location to another with four injection points each separated by 90 degrees. In some embodiments, less than a stoichiometric mole of H₂O₂ is provided compared to NO (mol: mol). In some embodiments, multiple sub-stoichiometric continuous infusions of H₂O₂ occurring at multiple locations along the absorption array; there are multiple stoichiometric continuous infusions of H₂O₂ occurring at multiple locations along the absorption array.

In some embodiments, the system described herein further comprise a fan. In certain embodiments, the fan is incorporated in-line along the exhaust duct. In some embodiments, the fan is placed either before a cooler within the system, or between the cooler and the absorber; and/or after the absorber. In some embodiments, the system described herein comprises an oxidant source such as ozone and the temperature of the engine exhaust at a point of ozone infusion is about 100° F., 110° F., 120° F., 130° F., 140° F., or 150° F. In some embodiments ozone (O₃) oxidation uses one of the following protocols, or a combination thereof: 1) a single-point, large (greater than stoichiometric based on NO) continuous infusion; 2) a single-point stoichiometric (based on NO) continuous infusion; and/or 3) multiple sub-stoichiometric continuous infusions occurring at multiple locations along the absorption array.

In one embodiment, the oxidant, such as O₃, infusion is performed using a single point greater than stoichiometric (based on NO) continuous infusion. The advantage of a large, single-point, continuous O₃ infusion is more rapid absorption of NOx due to the formation of water soluble and water reactive higher order nitrogen oxides. As illustrated in the equations below the higher order oxides of nitrogen N₂O₅, N₂O₃, and N₂O₄ react with water or alkaline water solutions.

NO₂+O₃→NO₃+O₂ (nitrate radical, strong oxidizer—unstable)

NO₂+NO₃→N₂O₅ (solid) [symbol] [NO₂⁺][NO₃⁻] (salt); reacts with water

N₂O₅+H₂O→2HNO₃

NO₂+NO [symbol] N₂O₃ (very soluble in water)

N₂O₃+2NaOH→2NaNO₂+H₂O

2NO₂ [symbol] N₂O₄ (liquid to 70° F.); reacts with water

N₂O₄+H₂O→HNO₂+HNO₃

In some embodiments, the single point comprises a single location with a single point of infusion. In some embodiments, the single point comprises a single location with multiple points of infusion. In some embodiments, the single point comprises multiple injection points. In some embodiments, the single point comprises a single location with a single inlet. In some embodiments, the single point comprises a single location with multiple inlets. In some embodiments, the single point comprises multiple inlets. In some embodiments, the single point comprises one or more locations around a 360 degree circumference a duct, pipe, tube, or other means of delivering gas and/or liquid from one location to another.

The table below shows that NOx absorbs more quickly with excess O₃.

TABLE 2
NOx levels with infusion of greater amounts of O₃

| | O₃ Added | NO | NO₂ |
|---|---|---|---|
| Measurement | (g/hr) | (ppm) | (ppm) |
| Baseline exhaust reading | 0 | 202 | 0 |
| O₃ added, Exhaust before Absorbers | 633 | 0 | 162 |
| O₃ added, Exhaust after one Absorber | 633 | 0 | 3.3 |

In one embodiment, the oxidant, such as O₃, infusion is performed using a single point, about a stoichiometric amount (based on NO) continuous infusion. The advantage of a stoichiometric, single-point, continuous O₃ infusion is the potential for lower ozone requirements and fewer side reactions. In some embodiments, the single point comprises a single location with a single point of infusion. In some embodiments, the single point comprises a single location with multiple points of infusion. In some embodiments, the single point comprises multiple injection points. In some embodiments, the single point comprises a single location with a single inlet. In some embodiments, the single point comprises a single location with multiple inlets. In some embodiments, the single point comprises multiple inlets. In some embodiments, the single point comprises one or more locations around a 360 degree circumference a duct, pipe, tube, or other means of delivering gas and/or liquid from one location to another.

In some embodiments, provided are multiple sub-stoichiometric continuous oxidant, such as O₃, infusions. An advantage of such infusions at multiple locations along the absorption array is the potential for still lower ozone requirements and fewer side reactions. Mixtures of NO and NO₂ are known to exhibit enhanced absorption into aqueous solutions due to the following reaction that yields a water soluble higher order oxide of nitrogen, N₂O₃.

NO+NO₂→N₂O₃

By removing some of the NO through this oxidation reaction-absorption pathway, less overall ozone is required as illustrated in the table below. The table below shows that NO₂ absorbs more completely with NO present.

TABLE 3
NOx levels with infusion of lower amounts of O₃

| | O₃ Added | NO | NO₂ |
|---|---|---|---|
| Measurement | (g/hr) | (ppm) | (ppm) |
| Baseline exhaust reading | 0 | 55 | 0 |
| O₃ added, Exhaust before Absorbers | 95 | 10 | 44 |
| O₃ added, Exhaust after Absorbers (3) | 95 | 6 | 1.9 |

In some embodiments, other oxidizing agents are used in addition to or in place of ozone. Illustrative examples of such oxidizing agents include but are not limited to halogen ions such as F, Cl⁻, Br⁻, F; chlorite, chlorate, perchlorate, and other analogous halogen compounds; hypochlorite and other hypohalite compounds, including household bleach (NaClO); hexavalent chromium compounds such as chromic and dichromic acids and chromium trioxide, pyridinium chlorochromate (PCC), and chromate/dichromate compounds; permanganate compounds such as potassium permanganate; sodium perborate; nitrous oxide (N₂O); silver oxide (Ag₂O); osmium tetroxide (OsO₄); tollens' reagent; 2,2′-Dipyridyldisulfide (DPS); and H₂O₂, other peroxides, and Ultraviolet irradiation.

Use of Oxidant to Control Emissions from Fossil Fuel Powered Boilers and Electricity Generation Facilities

In some embodiments, the ozone oxidation protocols described above are also useful in fossil fuel powered boilers and electricity generation facilities. In some embodiments, an oxidan such as ozone is used to convert NO to NO₂ to control emissions of NOx, SOx, and particulate matter by way of wet scrubbing from fossil fuel powered boilers and electricity generation facilities. In some embodiments, a gas fired boiler is used to generate energy and exhaust, the gas comprising natural gas and biogas.

### Mist Eliminator

In some embodiments, the system described herein provides a method of decreasing NOx emissions in an engine exhaust comprising contacting the engine exhaust from the combustion engine with the liquid waste stream from the biogas production unit to generate a treated exhaust with decreased NOx emissions. In some embodiments, the biogas production unit comprises an anaerobic digester that converts waste to a biogas stream.

In some embodiments, the system comprises (a) an absorber that is operatively connected to an engine exhaust source; (b) a combustion engine that produces energy and engine exhaust containing NOx; and (c) an oxidant source. In some embodiments, a biogas production unit converts waste to a biogas stream and a liquid waste stream, and a combustion engine utilizes the biogas stream to produce energy and an engine exhaust stream containing NOx whereby the engine exhaust gas stream is treated with an oxidant.

In some embodiments, the system provides a mist eliminator that is operatively connected to the oxidant source, and used after the oxidant infusion. In some embodiments, the mist eliminator comprises a polypropylene mesh material. In some embodiments, the polypropylene mesh material is a pad. In certain embodiments, the oxidant source is ozone or H₂O₂. In some embodiments, the mist eliminator comprises metals and/or plastics, including for example SS-304, 304L, 316, 316L, 430, Monel, Nickel, Copper, P.T.F.E. (Teflon), H.D.P.E., P.P. and the like, alloys and/or drawn or extruded plastic. The mist eliminator is not necessarily included for proper function of the systems, methods and devices described herein.

In some embodiments, the mist eliminator is a 4″ polypropylene mesh pad (style 16/96, 4 layers top; style 37/97, 2 layers bottom) that is used prior to O₃ infusion. In some embodiments, the use of a mist eliminator reduces the use of ozone and provides at least 20%, 25%, 30%, 35%, 40%, 45%, 50%, 55%, 60%, 65%, or at least 70% less per infusion site. For example, during a large, single-point, continuous O₃ infusion of 789 g/h residual O₃ concentrations of 342 ppm and 204 ppm were measured in the exhaust duct 15′ downstream from the site of infusion with and without the mist eliminator, respectively. The difference in measured O₃ concentration (138 ppm) is attributable to absorption and/or reaction loss due to moisture in the exhaust gas.

In some embodiments, a mist eliminator is used prior to any other oxidant infusion. Illustrative examples of such oxidizing agents include halogen ions such as F, Cl⁻, Br⁻, F; chlorite, chlorate, perchlorate, and other analogous halogen compounds; hypochlorite and other hypohalite compounds, including household bleach (NaClO); hexavalent chromium compounds such as chromic and dichromic acids and chromium trioxide, pyridinium chlorochromate (PCC), and chromate/dichromate compounds; permanganate compounds such as potassium permanganate; sodium perborate; nitrous oxide (N₂O); Silver oxide (Ag₂O); Osmium tetroxide (OsO₄); Tollens' reagent; 2,2′-Dipyridyldisulfide (DPS); and H₂O₂, other peroxides, and Ultraviolet irradiation.

In one aspect of the systems, methods and devices described herein, a reactor comprises a first absorber comprising a first liquid waste stream inlet, a first exhaust inlet and a first exhaust outlet and a second absorber comprising a second liquid waste stream inlet, a second exhaust inlet and a second exhaust outlet, provided that the first exhaust outlet of the first absorber is operatively connected to the second exhaust inlet of the second absorber, and provided that a mist eliminator is included in the second exhaust outlet of the second absorber. In some embodiments one or all absorbers contain mist eliminators in their respective exhaust outlets.

In one aspect of the systems, methods and devices described herein, a reactor comprises a first absorber comprising a first liquid waste stream inlet, a first exhaust inlet and a first exhaust outlet, a second absorber comprising a second liquid waste stream inlet, a second exhaust inlet and a second exhaust outlet and a third absorber comprising a third liquid waste stream inlet, a third exhaust inlet and a third exhaust outlet, provided that the first exhaust outlet of the first absorber is operatively connected to the second exhaust inlet of the second absorber, the second exhaust outlet of the second absorber is operatively connected to the third exhaust inlet of the third absorber, and provided that a mist eliminator is included in the third exhaust outlet of the third absorber. In some embodiments one or all absorbers contain mist eliminators in their respective exhaust outlets.

In one aspect of the systems, methods and devices described herein, a reactor comprises a first absorber comprising a first liquid waste stream inlet, a first exhaust inlet and a first exhaust outlet, a second absorber comprising a second liquid waste stream inlet, a second exhaust inlet and a second exhaust outlet, a third absorber comprising a third liquid waste stream inlet, a third exhaust inlet and a third exhaust outlet, and a fourth absorber comprising a fourth liquid waste stream inlet, a fourth exhaust inlet and a fourth exhaust outlet, provided that the first exhaust outlet of the first absorber is operatively connected to the second exhaust inlet of the second absorber, the second exhaust outlet of the second absorber is operatively connected to the third exhaust inlet of the third absorber, and the third exhaust outlet of the third absorber is operatively connected to the fourth exhaust inlet of the fourth absorber, and provided that a mist eliminator is included in the fourth exhaust outlet of the third absorber. In some embodiments one or all absorbers contain mist eliminators in their respective exhaust outlets.

In one aspect of the systems, methods and devices described herein, a reactor comprises a plurality of absorber each comprising a liquid waste stream inlet, an exhaust inlet and an exhaust outlet, provided that the exhaust outlet in the absorber in which the exhaust exits the reactor includes a mist eliminator. In some embodiments the plurality of absorbers are arranged in parallel and each exhaust outlet of each absorber includes a mist eliminator. In some embodiments one or all absorbers contain mist eliminators in their respective exhaust outlets.

Efficiency and Compliance with Regulatory Agencies of NOx, SOx and/or COx Emission Levels

A feature of the subject matter provided herein is a method of decreasing NOx emissions in an engine exhaust comprising contacting the engine exhaust from the combustion engine with the liquid waste stream from the biogas production unit to generate a treated exhaust with decreased NOx emissions.

Another feature of the subject matter provided herein are systems, methods and devices that are compliant with local, state and federal regulations that have established maximum levels of NOx, SOx, and/or COx emission limitations on emissions for biogas engines. In some embodiments, the methods, devices and systems provided herein reduce the level of NOx and SOx below the currently existing Best Available Control Technology (BACT) or Urea Injected Selective Catalytic Reduction (SCR) as designed by San Joaquin Valley Air Pollution Control District.

In some embodiments, the methods, devices and systems provided herein are compliant with the NOx emission levels required by the California Energy Commission (CEC) guidelines for certification of combined heat and power engine systems established pursuant to California's Waste, Heat and Carbon Emissions Reduction Act. In further or additional embodiments, the methods and devices and systems provided herein provide a decrease in emission levels over the conventional methods, devices, and/or systems, but are not compliant with the California Energy Commission (CEC) guidelines for certification of combined heat and power engine systems established pursuant to California's Waste, Heat and Carbon Emissions Reduction Act.

In some embodiments of the subject matter described herein, provided is a NOx level reduction (from baseline), i.e., the amount of NOx removed, of about 300 ppm, about 250 ppm, about 200 ppm, about 150 ppm, about 100 ppm, about 50 ppm, about 25 ppm, about 15 ppm, about 10 ppm, about 5 ppm, about 3 ppm, about 2 ppm, or about 1 ppm. In one example, provided is a NOx level reduction of a range of from about 25 ppm to less than about 3 ppm. In further or additional embodiments, provided is a SOx level reduction (form baseline), i.e., the amount of SOx removed, of about 1,000 ppm, about 750 ppm, about 600 ppm, about 585 ppm, about 550 ppm, about 500 ppm, about 400 ppm, about 300 ppm, about 200 ppm, about 100 ppm, about 50 pm, about 25 ppm, about 15 ppm, about 5 ppm, about 1 ppm, or about 0.1 ppm or less. For example, in one particular embodiment provided is a SOx level reduction of a range of from about 585 ppm to about 0.1 ppm.

In some embodiments, the subject matter provided herein provides treated exhaust with a NOx level that is undetectable using conventional methodology, or that is equal to or less than about 0.01, about 0.02, about 0.05, about 0.1, about 0.15, about 0.2, about 0.25, about 0.3, about 0.4, about 0.5, about 0.6, about 0.7, about 0.8, about 0.9, or about 1 grams per bhp-hr (or less than about 1 ppm, about 2 ppm, about 5 ppm, about 10 ppm, about 15 ppm, about 20 ppm, about 25 ppm, about 30 ppm, about 35 ppm, about 40 ppm, about 45 ppm, about 50 ppm, about 55 ppm, about 60 ppm, about 65 ppm, about 70 ppm, or about 75 ppm when adjusted to 15% O₂). In further or additional embodiments, the NOx level is equal to or less than about 0.01, 0.02, 0.03, 0.04, 0.05, 0.06, 0.07, 0.08, 0.09, or 0.1 lbs/MW-hr (or 0.5, 1, 2, 3, 4, 5, 6, 7, or 8 ppm when adjusted to 15% O₂). 0.6 grams per bhp-hr is converted to 1.55 lbs per MW-hr (as per CARB guidance: (http://www.arb.ca.gov/energy/dg/guidance/gappc.pdf). The NOx and SOx levels reported herein are determined when tested using a Testo 350XL portable hand-held combustion gas analyzer which uses electrochemical sensors for detection and is commercially available from Clean Air Engineering, Inc. (see company website at cleanair.com).

The instant inventors have shown that at a demonstration site, the full exhaust stream of the subject matter described herein has a pollutant profile of approximately 50-100 ppm NOx (including intervals of 50, 55, 60, 65, 70, 75, 80, 85, 90, 95, and 100 ppm) and 400-1000 ppm SOx (including intervals of 400, 450, 500, 550, 600, 650, 700, 750, 800, 850, 900, 950, and 1,000 ppm) when tested using a Testo 350XL portable hand-held combustion gas analyzer.

In some embodiments of the system described herein, the system design is a modular format that decreases both the footprint and the cost of the quencher, cooler and absorption stages while maintaining flexibility in both scale and performance.

While preferred embodiments of the presently disclosed subject matter have been shown and described herein, it will be obvious to those skilled in the art that such embodiments are provided by way of example only. Numerous variations, changes, and substitutions will now occur to those skilled in the art without departing from the subject matter described herein. It should be understood that various alternatives to the embodiments described herein may be employed in practice. It is intended that the following claims define the scope of the invention and that methods and structures within the scope of these claims and their equivalents be covered thereby.

### EXAMPLES

The following specific examples are to be construed as merely illustrative, and not limitative of the remainder of the disclosure in any way whatsoever.

### Example 1

### Installation of the Methods, Devices and Systems in a Dairy

In the following example, the system is installed at a dairy in California and it is demonstrated that NOx and SOx emissions are reduced in the exhaust stream of a 300 kW Guascor engine. The system is installed at a 3,500 head flushed lane California based dairy as illustrated in FIG. 11. The site is equipped with a stirred, covered lagoon type anaerobic digester (AD) of approximately 30 million gallons. An additional uncovered storage lagoon of identical size is installed adjacent to the digester and serves as a storage location for AD effluent. Effluent from this storage lagoon is pumped into wastewater tanks located at the head of each feeding lane and then used to flush the lanes back into the digester two times per day.

A biogas stream of 75-150 scfm is produced by the digester in FIG. 11. The biogas is passed through a coiled-loop chiller to condense and remove water prior to combustion. The gas has an average composition of approximately 71.3 mole % methane, 24.48% CO₂, 2.78% N₂, and 1.08% H₂S when sampled after the chiller. The H₂S concentration varies between 6,000 ppm and 15,000 ppm. No sulfide removal is employed prior to combustion.

The full biogas stream is used to power a 300 kW Guascor lean burn CHP biogas engine. The manufacturer's engine specifications document lists 1948 acfm as the peak exhaust gas volume for this engine at 743° F. The raw untreated exhaust gas is measured by the authors to have a pollutant profile of approximately 50-100 ppm NOx and 400-1000 ppm SOx. All exhaust gas composition data is acquired using a Testo 350XL portable hand held combustion gas analyzer.

The system, as illustrated, for example, in FIG. 6 below, incorporates a modular multi-stage wet-scrubbing system that utilizes wastewater from an anaerobic digester with a unique NO oxidation strategy. The system is non-catalytic; and thus there is no need for biogas clean-up (siloxane and H₂S removal) prior to combustion for effective emission reduction purposes.

In the non-limiting examples described here, the first stage of the system is the Quencher/Cooler, which simultaneously cools and saturates the exhaust with water. The Quencher/Cooler cools the exhaust to about 100° F. and thus conditions the exhaust for rapid oxidation using ozone. Following NO oxidation, the exhaust enters the Absorber Array. Within the first absorber, 50-75% of the NOx are removed as the NO₂ absorbs and reacts within the absorber with the anaerobic digester effluent. The NOx level in the exhaust stream is similarly reduced by each subsequent absorber stage before leaving the stack containing the targeted levels of SOx and NOx emissions.

Using the system, both NOx and SOx emissions are reduced to CARB levels of 2-3 ppm. Further, NOx is reduced to levels below the current Best Available Control Technology or BACT (Urea Inject Selective Catalytic Reduction or SCR) as designated by San Joaquin Valley Air Pollution Control District (Table 4). The current NOx BACT level is 9-11 ppm adjusted to 15% O₂.

TABLE 4

| | Before | After |
|---|---|---|
| NOx | 25 ppm* | <3 ppm* |
| SOx | 585 ppm | <0.1 ppm |

*NOx Levels are adjusted to 15% O₂ basis.

A. Quencher and Cooler Stages

Hot biogas engine exhaust is saturated with water vapor and cooled prior to the oxidation and wet scrubbing steps. The exhaust is saturated with either plant water or water from a primary/secondary clarifier within the Quencher. Some initial cooling of the exhaust also takes place within the Quencher. The saturated exhaust stream is then further cooled within the Cooler. This is accomplished through the use of either digester effluent or primary/secondary clarifier water within the Cooler. All SO₂ is effectively removed by the Cooler as illustrated by Table 5 below.

TABLE 5

| SO₂ Before | SO₂ After | | |
|---|---|---|---|
| Cooler | Cooler | Engine Output | Cooler Dimensions |
| 726 ppm | 0 ppm | 150 kW/600 acfm | 8′ × 2′ Lanpac 2.3″ |

B. Multi-Stage Wet Scrubber (Absorber Array):

Three absorbers are constructed for the demonstration project (FIG. 1). The first absorber is three feet in diameter and contains 13.5 vertical feet of 2.3″ LANPAC® polypropylene packed media. The second and third absorbers are each three feet in diameter and contain 8 vertical feet of 2.3″ LANPAC® polypropylene packed media. Several types of packed media are tested including ceramic saddles, and larger LANPAC-XL® polypropylene media each with varying effective surface area, void fraction and pressure drop. Among all the packing media tested, 2.3″ LANPAC® polypropylene is determined to have the best performance. AD effluent is introduced into the top of each absorber at a rate of 20-60 gallons per minute, and 50-75% of the NO₂ is absorbed into and reacted with the AD effluent at each stage.

C. Nitrogen Level in Wastewater

As NOx absorbs into the wastewater, the nitrogen level theoretically increases, but not by amounts that are measured. For example, the increased nitrogen for a typical 10 mgd municipal wastewater treatment plant operating a 1 MW lean burn biogas engine is calculated as follows. The untreated NOx level in a lean burn biogas engine is approximately 0.6 grams per bhp-hr (or 40 ppm at 15% O₂), and the CARB standard is 0.07 Lbs. per MW-hr (or 2-3 ppm at 15% O₂). 0.6 grams per bhp-hr are converted to 1.55 Lbs. per MW-hr (as per CARB guidance: http://www.arb.ca.gov/energy/dg/guidance/gappc.pdf). If the system is installed on a 1 MW lean burn biogas fired engine operating 24 hours per day, the untreated NOx would be 1.55×24=37.2 Lbs of NOx per day. The treated NOx level would be 0.07×24=1.68 Lbs. per day.

Assuming the following:

Molecular weight of nitrogen: 46

Molecular weight of oxygen: 16

Molecular weight of NOx (as NO₂): 78

Percent nitrogen in NOx (as NO₂): 59%

59%×(37.2-1.68)=21 Lbs. of nitrogen per day

Therefore, if the system is installed on a 1 MW lean burn engine operating 24 hours per day, 21 Lbs. of nitrogen per day would be introduced into the head works of a 10 million gallon per day municipal wastewater treatment facility. One gallon of water weighs 8.34 Lbs., and thus 21 Lbs. of nitrogen would be added to approximately 83.4 million Lbs. of sewage. The change in nitrogen level would be too small to be accurately measured.

### Example 2

### Agents Used in the Reactor: Microbial

The absorption of NO₂ into, and subsequent reaction with, water produces nitrate ions. Nitrate reducing bacteria are known anaerobes and aerobes expected to be present within vast bacterial consortium present in the anaerobic digester (AD) effluent. These microbes are capable of reducing nitrate to nitrogen gas. Examples include Thiobacillus denitrificans, Micrococcus denitrificans, Paracoccus denitrificans and Pseudomonas.

### Example 3

### Use of Oxidant to Control Emissions from Fossil Fuel Powered Boilers and Electricity Generation Facilities

The ozone oxidation protocols provided herein are also useful in fossil fuel powered boilers and electricity generation facilities. An oxidant, such as ozone is used to convert NO to NO_{2 to control emissions of NOx, SOx, and particulate matter from fossil fuel powered boilers and electricity generation facilities.}

### Example 4

### Fan

An in-line fan is incorporated to mitigate back pressure on the biogas engine. The fan is placed in-line along the exhaust duct either before the Quencher/Cooler assembly, between the Cooler and the Absorbers, or after the Absorbers. Data is obtained with the fan located both between the Cooler and Absorbers and after the Absorbers. Placement of the fan between the Cooler and the Absorber is preferable and superior to placement after the Absorber. For example, single absorber performance is increased 6% by moving the exhaust fan from after the Absorber to between the Cooler and the Absorber. This increased absorption efficiency is believed to result from the slight positive pressure that results from pushing the exhaust through the Absorber rather than the negative pressure resulting from pulling it through.

### Example 5

### Mist Eliminators

Mist eliminators are used prior to any O₃ infusion site to conserve and minimize the use of O₃. By incorporating a 4″ polypropylene mesh pad (style 16/96, 4 layers top; style 37/97, 2 layers bottom) mist eliminator prior to O₃ infusion, as much as 40% less ozone is used per infusion site. For example, during a large, single-point, continuous O₃ infusion of 789 g/h residual O₃ concentrations of 342 ppm and 204 ppm are measured in the exhaust duct 15′ downstream from the site of infusion with and without the mist eliminator, respectively. The difference in measured O₃ concentration (138 ppm) is attributable to absorption and/or reaction loss due to moisture in the exhaust gas. Mist eliminators are also used prior to any other oxidant infusion sites.

### Example 6

### H₂S and Siloxane Removal Systems

The system described herein further comprises H₂S and siloxane removal systems. On inclusion of these systems, a catalytic stage that oxidizes approximately 50% of the NO to NO₂ and approximately 95% of the CO to CO₂ is used in conjunction with the system.

### Example 7

### Modular System

The system described herein is also modularly configured, such that, as a non-limiting example, one or more quenchers, coolers, absorbers, fans, mist eliminators, engines, boilers, fans, H₂S removal systems, siloxane removal systems and other system components can be added or removed. The modularity provides flexibility to keep up with increasingly stringent regulations for reducing NOx emissions. The modularity also affords scalability such that an increase in the amount of exhaust containing NOx, SOx and COx, resulting from an increase in the size or number of biogas production units and/or installation of additional devices constructed to utilize a biogas stream, is processed by the system and generate treated exhaust with decreased NOx, SOx, and COx emissions

### Example 8

### Installation of the Methods, Devices and Systems in a Wastewater Treatment Facility

Many municipal wastewater treatment plants utilize anaerobic digestion as part of their sewage treatment process. The US EPA estimated in an October 2011 report that there are currently 104 wastewater treatment plants producing 190 MW of biogas powered electricity. The thermal and electrical energy is typically used to help run processes at the wastewater treatment plant. Additionally, there are 1,351 wastewater treatment plants currently flaring biogas that could be combusted in a heat and electricity producing biogas engine.

A typical municipal wastewater treatment plant will have perhaps 10 mgd of influent. After screening and grit removal, collected sludge is pumped into heated tanks (digesters). Anaerobic bacteria thrive in the digesters, and convert raw sewage sludge to an inert material (e.g. digested sludge), methane gas and carbon dioxide. The anaerobic digestion process produces gas (e.g. methane) as a byproduct which is used for heating digesters and generation of electrical power for in-the plant use. Before combustion, biogas produced by wastewater treatment plants is frequently conditioned, whereby moisture, sulfur compounds and siloxanes are removed from the biogas stream.

In this example, the digested sludge from the digesters is transferred to centrifuges in the dewatering building. The centrifuges separate the digested sludge solids (cake) from the liquid (centrate). Sludge cake is discharged to a system of collection screw conveyers for transfer to sludge hauling trucks. The sludge cake is then hauled away for off-site disposal.

A cogeneration system is used to recover the energy available from the digester gas for plant use. Recovery of the energy from the digester gas is accomplished by feeding biogas produced by the digesters to engine-generators for electrical power production and generation of hot water for digester heating through recovery of a portion of the waste heat from the power generation equipment.

An embodiment of the system, methods and devices described herein is being installed at a California based municipal wastewater treatment plant that has two 848 kW biogas fired engines operating. The exhaust stream from one of the existing lean burn biogas engines has a volume estimated to be 2,500 scfm with NOx levels of approximately 90 ppm at 15% O₂. There is currently no emissions control installed on the existing exhaust stream.

The exhaust will be diverted to a concrete pad next to the building housing the electrical generators. An oxidation catalyst from Johnson Matthey will be installed to oxidize about 50% of the NO to NO₂, 90% of the CO to CO₂, and also oxidize a significant portion of any VOC's that may exist within the exhaust stream. After the oxidation catalyst, the exhaust stream will be quenched and cooled to less than 120° F. using plant water available onsite.

After the exhaust is cooled down, NO will be fully oxidized to NO₂ using ozone injected into the exhaust stream after a cooler and prior to a blower installed within the system.

The ozone will be produced onsite using an oxygen generator which feeds a series of ozone generators. After the NO is fully oxidized to NO₂ the exhaust will be pushed into a series of absorbers by a variable speed blower designed to minimize back pressure on the engine's exhaust stack. Within the absorbers, the exhaust will flow countercurrent to liquid centrate. Lanpac 2.3″ packing media will be used in the absorbers where NO₂ will absorb into and react with constituents within the centrate

Centrate is produced onsite when sludge from the anaerobic digesters is dewatered using centrifuges. The liquid portion of the sludge is centrate, which is stored in a tank prior to being fed into the headwork of the wastewater treatment plant. Centrate will gravity flow at about 50 gpm or less from the centrate tank to a sump located next to the absorbers, and a sump pump will be used to circulate centrate into the absorbers, and will gravity flow back to the sump area. Centrate will be circulated from the sump area through the absorbers and back to the sump area at a rate up to 300 gpm, and liquid centrate will gravity flow from the sump area to the headwork of the wastewater treatment plant at a rate of about 50 gpm.

The absorber volume and centrate flow volume will be adjusted such that the resulting exhaust stream from the biogas engine will have a decreased CO, NOx and VOC emission complying with regulations set by the California Air Resource Board (NOx 2-3 ppm), San Joaquin Valley Air Pollution Control District's rule 4702 (NOx 9-11 ppm), and South Coast Air Quality Management District's rule 1110.2 (NOx 11 ppm, CO 250 ppm, VOC's: 30 ppm).

The examples and embodiments described herein are for illustrative purposes only and various modifications or changes are included within the spirit and purview of this application and scope of the appended claims.

1.-131. (canceled)

132. A system comprising:
(a) a reactor comprising: a first absorber comprising a first liquid waste stream inlet, a first exhaust inlet, and a first exhaust outlet; and a second absorber comprising a second liquid waste stream inlet, a second exhaust inlet, and a second exhaust outlet;
(b) a biogas production unit constructed so as to convert waste to a biogas stream and a liquid waste stream, the biogas production unit comprising a biogas stream outlet and a liquid waste stream outlet, the liquid waste stream outlet operatively connected to the first liquid waste stream inlet of the first absorber and operatively connected to the second liquid waste stream inlet of the second absorber; and
(c) a device constructed to utilize the biogas stream to produce energy and an exhaust comprising a gas, the device comprising: a biogas stream inlet operatively connected to the biogas stream outlet of the biogas production unit; and a device exhaust outlet, provided that the device exhaust outlet is operatively connected to the first exhaust inlet of the first absorber, and the first exhaust outlet of the first absorber is operatively connected to the second exhaust inlet of the second absorber, or provided that the device exhaust outlet is operatively connected to the first exhaust inlet of the first absorber and the second exhaust inlet of the second absorber;
provided that the reactor is constructed so as to treat exhaust so as to decrease or reduce emissions or pollutants by contacting the exhaust with the liquid waste stream from the biogas production unit.

133. The system of claim 132, provided that the exhaust comprises a gas comprising one or more of NOx, SOx, or COx.

134. The system of claim 132, provided that the device comprises one or more of a biogas engine, an internal combustion engine, a gas fired boiler, a turbine, or a microturbine.

135. The system of claim 132, provided that the device further comprises a natural gas stream inlet, and provided that the device is constructed so that the natural gas stream and the biogas stream are utilized to produce energy.

136. The system of claim 132, further comprising a quencher operatively connected to the exhaust outlet of the device.

137. The system of claim 136, further comprising a cooler operatively connected to the quencher.

138. The system of claim 137, further comprising a fan operatively connected to one or more of the quencher, the cooler, the first absorber, and the second absorber.

139. The system of claim 132, provided that the reactor further comprises a mist eliminator operatively connected to the first or second absorber so as to access the exhaust.

140. The system of claim 137, further comprising a mist eliminator operatively connected to the cooler so as to access the exhaust.

141. The system of claim 132, further comprising an oxidant injection site operatively connected to the system so as to access to the exhaust, the oxidant injection site comprising one or more inlets.

142. The system of claim 132, provided that oxidant is provided to the system so as to access the exhaust.

143. A reactor comprising: a first absorber comprising a first liquid waste stream inlet, a first exhaust inlet, and a first exhaust outlet; and a second absorber comprising a second liquid waste stream inlet, a second exhaust inlet, and a second exhaust outlet, the reactor operatively connected to:
(a) a biogas production unit constructed so as to convert waste to a biogas stream and a liquid waste stream, the biogas production unit comprising a biogas stream outlet and a liquid waste stream outlet, the liquid waste stream outlet operatively connected to the first liquid waste stream inlet of the first absorber and operatively connected to the second liquid waste stream inlet of the second absorber; and
(b) a device constructed to utilize the biogas stream to produce energy and an exhaust comprising a gas, the device comprising: a biogas stream inlet operatively connected to the biogas stream outlet of the biogas production unit; and a device exhaust outlet, provided that the device exhaust outlet is operatively connected to the first exhaust inlet of the first absorber, and the first exhaust outlet of the first absorber is operatively connected to the second exhaust inlet of the second absorber, or provided that the device exhaust outlet is operatively connected to the first exhaust inlet of the first absorber and the second exhaust inlet of the second absorber;
provided that the reactor is constructed so as to treat exhaust so as to decrease or reduce emissions or pollutants by contacting the exhaust with the liquid waste stream from the biogas production unit.

144. The reactor of claim 143, further comprising a third absorber operatively connected to the first absorber, the second absorber, or the device, the third absorber comprising a third liquid waste stream inlet, a third exhaust inlet, and a third exhaust outlet.

145. The reactor of claim 144, further comprising a fourth absorber operatively connected to the first absorber, the second absorber, the third absorber, or the device, the fourth absorber comprising a fourth liquid waste stream inlet, a fourth exhaust inlet, and a fourth exhaust outlet.

146. The reactor of claim 144, provided that the first absorber, the second absorber and the third absorber are aligned in series, in parallel or in a combination of in series and in parallel.

147. The reactor of claim 145, provided that the first absorber, the second absorber the third absorber and the fourth absorber are aligned in series, in parallel or in a combination of in series and in parallel.

148. The reactor of claim 143, provided that the first absorber, the second absorber, or the first absorber and the second absorber comprises a nitrate reducing bacterium, the nitrate reducing bacterium comprising Thiobacillus denitrificans, Micrococcus denitrificans Paracoccus denitrificans or Pseudomonas.

149. The reactor of claim 143, further comprising a fan operatively connected to one or more of the first absorber and the second absorber.

150. The reactor of claim 143, provided that the reactor further comprises a mist eliminator operatively connected to the reactor so as to access the exhaust.

151. A method to treat exhaust comprising:
(a) providing a system comprising:
(i) a reactor comprising: a first absorber comprising a first liquid waste stream inlet, a first exhaust inlet and a first exhaust outlet; and a second absorber comprising a second liquid waste stream inlet, a second exhaust inlet and a second exhaust outlet;
(ii) a biogas production unit constructed so as to convert waste to a biogas stream and a liquid waste stream, the biogas production unit comprising a biogas stream outlet and a liquid waste stream outlet, the liquid waste stream outlet operatively connected to the first liquid waste stream inlet of the first absorber and operatively connected to the second liquid waste stream inlet of the second absorber; and
(iii) a device constructed to utilize the biogas stream to produce energy and an exhaust comprising a gas, the device comprising: a biogas stream inlet operatively connected to the biogas stream outlet of the biogas production unit; and a device exhaust outlet, provided that the device exhaust outlet is operatively connected to the first exhaust inlet of the first absorber, and the first exhaust outlet of the first absorber is operatively connected to the second exhaust inlet of the second absorber, or provided that the device exhaust outlet is operatively connected to the first exhaust inlet of the first absorber and the second exhaust inlet of the second absorber; and
(b) delivering waste to the biogas production unit;
(c) generating exhaust comprising the gas using the device; and
(d) contacting the exhaust with the liquid waste stream so as to treat exhaust so as to decrease or reduce emissions or pollutants.
